(19) United States
(12) Patent Application Publication
(10) Pub. No.: US 2012/0094958 A1
(43) Pub. Date: Apr. 19, 2012

(54) QUINOLINES AS INHIBITORS OF FARNESYL PYROPHOSPHATE SYNTHASE

(75) Inventors: Rene Amstutz, Basel (CH); Guido Bold, Basel (CH); Simona Cotesta, Basel (CH); Wolfgang Jahnke, Basel (CH); Andreas Marzinzik, Basel (CH); Juliane Constanze Desiree Hartwieg, Basel (CH); Silvio Ofner, Basel (CH); Frédérik Stauffer, Basel (CH); Johann Zimmermann, Basel (CH)

(73) Assignee: NOVARTIS AG, Basel (CH)

(21) Appl. No.: 12/918,880

(22) PCT Filed: Feb. 26, 2009
(86) PCT No.: PCT/EP2009/052314

§ 371 Date: Apr. 20, 2011

(30) Foreign Application Priority Data
Feb. 28, 2008 (EP) 08152046.2

(51) Int. Cl. A61K 31/47; C07D 401/10; C07F 9/60; C07F 9/6558; C07D 417/12; C07F 1/02; A61K 31/4709; A61K 31/4725; A61K 31/675; A61P 19/08; A61P 35/00; A61P 3/06; A61P 31/00; A61P 35/04; C07D 215/48

(52) U.S. Cl. 514/82; 546/170; 546/141; 546/157; 546/23; 546/21; 546/169; 546/167; 514/311; 514/314; 514/307; 514/309; 544/243; 514/312

(57) ABSTRACT

The invention relates to a compound of formula (I) wherein the substituents are as described in the specification, which are useful as farnesyl pyrophosphate synthase modulators, e.g. in the treatment of proliferative diseases, to methods of manufacturing such compounds and to intermediates thereof.

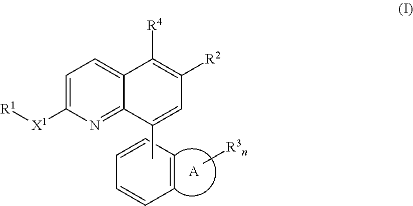

The invention relates to novel Bi-Aryl derivatives of formula (I) given below; to Bi-Aryl derivatives of the formula (I′) given below as medicament; to the use of compounds of formula (I) and (I′) for use in the treatment of a disorder that depends on the activity of farnesyl pyrophosphate synthase (FPPS), especially a proliferative disease and/or a cholesterol biosynthesis related disorder; to pharmaceutical preparations, optionally in the presence of a combination partner; to the use of such preparations in the treatment of a disease or disorder, in particular a disorder as disclosed herein; to the manufacture of a compound of formula (I) and (I′); to the manufacture of a pharmaceutical preparation containing a compound of formula (I) and/or (I′).

It is known that FPPS is a key branchpoint enzyme in the mevalonate pathway. Hence, FPPS is recognized as an important drug target. It is anticipated that new FPPS inhibitors would have therapeutic potential for the treatment of bone diseases, in oncology, for the treatment of elevated cholesterol levels and as anti-infectives.

It is further known that certain Bi-Aryl derivatives, in particular from the group of substituted quinoline2,4-dicarbxyles, are competitive inhibitors of VGLUT and might thus be suitable candidates for the treatment of CNS related diseases; see J. Med. Chem., 2002, 2260ff.

It is thus an aim of the present invention to provide novel FPPS inhibitors and methods of inhibition of FPPS-dependent disorders, in particular with advantageous pharmacological properties, such as enhanced efficacy, tolerability, oral bioavailability and/or pharmacokinetics.

Surprisingly, it has now been found that Bi-Aryl derivatives of the present invention show FPPS inhibition although they are not bisphosphonates, and that they are appropriate for the treatment of diseases that depend on FPPS activity, especially against tumor and cancer diseases of soft and hard tissues, especially metastasis, e.g. bone metastasis, or as cholesterol-lowering agents. In addition, a large number of novel compounds of this class have been found that are FPPS inhibitors.

The invention may be more fully appreciated by reference to the following description, including the following glossary of terms and the concluding examples. For the sake of brevity, the disclosures of the publications cited in this specification are herein incorporated by reference. As used herein, the terms “including”, “containing” and “comprising” are used herein in their open, non-limiting sense.

Any formula given herein is intended to represent compounds having structures depicted by the structural formula as well as certain variations or forms. In particular, compounds of any formula given herein may have one or more asymmetric centers or other elements of asymmetry, and therefore exist in different enantiomeric forms. If at least one asymmetrical carbon atom is present in a compound of the formula (I), such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers. Additionally, any formula given herein is intended to represent hydrates, solvates, polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. Various isotopically labeled compounds of the present invention, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a. readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

The following general definitions shall apply in this specification, unless otherwise specified:

“A” compound, “a” salt, “a” disorder, “a” disease or the like preferably means “one or more” compounds, salt, disorders, diseases or the like. Where the plural form (e.g. compounds, salts) is used, this includes the singular (e.g. a single compound, a single salt). “A compound” does not exclude that (e.g. in a pharmaceutical formulation) more than one compound of the formula (I) (or a salt thereof) is present.

“Treatment” or “therapy” refers to the prophylactic or preferably therapeutic (including but not limited to palliative, curing, symptom-alleviating, symptom-reducing, FPPS-activity-regulating and/or FPPS-inhibiting) treatment of said diseases/disorder, especially of the diseases/disorders mentioned herein.

“Obtainable by” can preferably be replaced with “obtained by”.

Where the term “comprising” is used, this is intended to mean that the component, components, action, actions, feature or features mentioned or enumerated thereafter may be fulfilled not only alone, but that also one or more other components and/or features (e.g. other additives, other actions) may be present in addition to those specifically mentioned. This is in contrast to the term “containing” or “consisting of” which here mean that no other components or features are included except for those specifically mentioned after such an expression and thus denote a complete enumeration/representation of features and/or components. Wherever “comprising” is used, this may (independently of other occurrences) be replaced by the narrower term “consisting of” or (in case of processes or methods) by “containing the step of”, where possible and expedient, thus leading to specific and preferred embodiments of the invention.

“Salts” (which, what is meant by “or salts thereof” or “or a salt thereof”), can be present alone or in mixture with free compound of the formula (I)) are preferably pharmaceutically acceptable salts. Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

“Esters” (which, what is meant by “or ester thereof” or “or a ester thereof”), can be present alone or in mixture with free compound of the formula (I)) are preferably pharmaceutically acceptable esters. Such esters are formed, for example, with alcohols from compounds of formula (I) with an acid group, such as —CO₂H, —P(O)(OH)₂ and the like. Suitable alcohols are, for example, ethanol, methanol, benzylalcohol. For therapeutic use, only pharmaceutically acceptable esters or free compounds are employed (where applicable in the form of pharmaceutical preparations), and these are therefore preferred. In view of the close relationship between the novel compounds in free form and those in the form of their esters, including those esters that can be used as intermediates, for example in the purification or identification of the novel compounds, any reference to the free compounds hereinbefore and hereinafter is to be understood as referring also to the corresponding esters, as appropriate and expedient.

“Halo” (or halogen) is preferably fluoro, chloro, bromo or iodo, most preferably F, Cl or Br.

In unsubstituted or substituted “alkyl”, alkyl (also in alkoxy or the like) preferably has up to 20, more preferably up to 12 carbon atoms, is linear or branched, and is more preferably lower alkyl, such as C₁-C₆-alkyl, especially C₁-C₄-alkyl. Substituted alkyl is preferably C₁- to C₂₀-alkyl, more preferably lower alkyl, that can be linear or branched one or more times (provided the number of carbon atoms allows this), e.g. methyl, ethyl, propyl, n-butyl, sec-butyl, isobutyl, tert-butyl, 2,2-dimethylpropyl, 1,2,2-trimethylpropyl, 1-ethyl-propyl, and that is substituted by one or more, preferably up to three, substitutents independently selected from the group consisting of unsubstituted or substituted heterocyclyl as described below, especially pyrrolidinyl, such as pyrrolidino, oxopyrrolidinyl, such as oxopyrrolidino, C₁-C₇-alkyl-pyrrolidinyl, 2,5-di-(C₁-C₇alkyl)pyrrolidinyl, such as 2,5-di-(C₁-C₇alkyl)-pyrrolidino, tetrahydrofuranyl, thiophenyl, C₁-C₇-alkylpyrazolidinyl, pyridinyl, C₁-C₇-alkylpiperidinyl, piperidino, piperidino substituted by amino or N-mono- or N,N-di-[lower alkyl, phenyl, C₁-C₇-alkanoyl and/or phenyl-lower alkyl)-amino, unsubstituted or N-lower alkyl substituted piperidinyl bound via a ring carbon atom, piperazino, lower alkylpiperazino, morpholino, thiomorpholino, S-oxo-thiomorpholino or S,S-dioxothiomorpholino; unsubstituted or substituted aryl as defined below, especially phenyl, naphthyl, mono- to tri-[C₁-C₇alkyl, halo and/or cyano]-phenyl or mono- to tri-[C₁-C₇-alkyl, halo and/or cyano]-naphthyl; unsubstituted or substituted cycloalkyl as defined below, especially C₃-C₈-cycloalkyl, mono- to tri-[C₁-C₇-alkyl and/or hydroxy]-C₃-C₈-cycloalkyl; halo (e.g. in trifluoromethyl), hydroxy, lower alkoxy, lower-alkoxy-lower alkoxy, (lower-alkoxy)-lower alkoxy-lower alkoxy, halo-C₁-C₇-alkoxy, tri-(C₁-C₇-alkyl)silyl-C₁-C₇-alkoxy-C₁-C₇-alkoxy, phenoxy, naphthyloxy, phenyl- or naphthyl-lower alkoxy; amino-lower alkoxy, lower-alkanoyloxy, benzoyloxy, naphthoyloxy, nitro, cyano, formyl (CHO), carboxy, lower alkoxy carbonyl, e.g.; phenyl- or naphthyl-lower alkoxycarbonyl, such as benzyloxycarbonyl; C₁-C₇-alkanoyl, such as acetyl, benzoyl, naphthoyl, carbamoyl, N-mono- or N,N-disubstituted carbamoyl, such as N-mono- or N,N-di-substituted carbamoyl wherein the substitutents are selected from lower alkyl and hydroxy-lower alkyl; amidino, guanidino, ureido, mercapto, lower alkylthio, phenyl- or naphthylthio, phenyl- or naphthyl-lower alkylthio, lower alkyl-phenylthio, lower alkyl-naphthylthio, halogen-lower alkylmercapto, lower alkylsulfinyl, phenyl- or naphthyl-sulfinyl, phenyl- or naphthyl-lower alkylsulfinyl, lower alkyl-phenylsulfinyl, lower alkyl-napthylsulfinyl, sulfo, lower alkanesulfonyl, phenyl- or naphthyl-sulfonyl, phenyl- or naphthyl-lower alkylsulfonyl, alkylphenylsulfonyl, halogen-lower alkylsulfonyl, such as trifluoromethanesulfonyl; sulfonamido, benzosulfonamido, azido, azido-C₁-C₇-alkyl, especially azidomethyl, amino, amino-C₁-C₇ alkyl, especially aminomethyl, N-mono- or N,N-di-[lower alkyl, phenyl, C₁-C₇-alkanoyl and/or phenyl-lower alkyl)-amino or N-mono- or N,N-di-[lower alkyl, phenyl, C₁-C₇alkanoyl and/or phenyl-lower alkyl)-aminomethyl; where each phenyl or naphthyl (also in phenoxy or naphthoxy) mentioned above as substituent or part of a substituent of substituted alkyl (or also of substituted aryl, heterocyclyl etc. mentioned herein) is itself unsubstituted or substituted by one or more, e.g. up to three, preferably 1 or 2, substituents independently selected from halo, especially fluoro, chloro, bromo or iodo, halo-lower alkyl, such as trifluoromethyl, hydroxy, lower alkoxy, azido, amino, N-mono- or N,N-di-(lower alkyl, phenyl, naphthyl, C₁-C₇-alkanoyl, phenyl-lower alkyl and/or naphthyl-lower alkyl)-amino, nitro, formyl (CHO), carboxy, lower-alkoxycarbonyl carbamoyl, cyano and/or sulfamoyl. In the case of R¹ in formula (I), unsubstituted or substituted alkyl is preferably C₁-C₇-alkyl, such as methyl or ethyl, halo-C₁-C₇-alkyl, such as halomethyl, hydroxyl-C₁-C₇-alkyl, such as hydroxymethyl, amino-C₁-C₇-alkyl, such as aminomethyl, or carboxy-C₁-C₇-alkyl, such as carboxymethyl.

Unsubstituted or substituted “alkenyl” is preferably C₂-C₂₀-alkenyl, more preferably C₂-C₁₂-alkenyl, yet more preferably C₂-C₇-alkenyl, which is linear or branched and includes one or more double bonds. The substituents are preferably one or more, especially up to three, substituents independently selected from those mentioned for substituted alkyl, preferably with the proviso that substituents with active hydrogen (such as amino or hydroxyl) can also be present in tautomeric form (as keto or imino compounds) or are excluded from the substituents where the stability is too low.

Unsubstituted or substituted “alkynyl” is preferably C₂-C₂₀-alkynyl, more preferably C₃-C₁₂-alkynyl, yet more preferably C₃-C₇-alkynyl, which is linear or branched and includes one or more triple bonds. The substituents are preferably one or more, especially up to three, substituents independently selected from those mentioned for substituted alkyl, preferably with the proviso that substituents with active hydrogen (such as amino or hydroxyl) can also be present in tautomeric form (as keto or imino compounds) or are excluded from the substituents where the stability is too low.

Unsubstituted or substituted “alkandiyl” is preferably a straight-chain or branched-chain alkandiyl group bound by two different Carbon atoms to the moiety, it preferably represents a straight-chain or branched-chain O₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl. If such alkandiyl is interrupted by one or more groups, e.g. —O—, —C(O)—, —N(H)—, this includes groups such as —CH₂—C(O)—; —CH₂—C(O)—N(H)—; —CH₂—N(H)—C(O)—; —C(O)—CH₂—N(H)— and the like.

Unsubstituted or substituted “alkendiyl” is preferably a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—. Alkendiyl may be substituted or unsubstituted. If such alkendiyl is interrupted by one or more groups, e.g. —O—, —C(O)—, —N(H)—, this includes groups such as —CH═CH—CH₂—C(O)—; —CH═CH—CH₂—C(O)—N(H)— and the like.

In unsubstituted or substituted “aryl”, aryl is preferably an unsaturated carbocyclic system of not more than 20 carbon atoms, especially not more than 16 carbon atoms, is preferably mono-, bi- or tri-cyclic, e.g. phenyl, naphthyl, phenanthrenyl or fluorenyl, which is unsubstituted or, as substituted aryl, substituted preferably by one or more, preferably up to three, e.g. one or two substituents independently selected from those mentioned above for substituted alkyl, and from alkenyl. Preferably, the substituents are independently selected from the group consisting of C₁-C₇-alkyl, such as methyl, hydroxyl-C₁-C₇-alkyl, such as hydroxymethyl, halo, such as fluoro, chloro, bromo or iodo, hydroxyl, C₁-C₇-alkoxy, such as methoxy, halo-C₁-C₇-alkoxy, such as trifluoromethoxy, amino, C₁-C₇-alkanoylamino, such as acetylamino, amino-alkyl, such as aminomethyl, N-mono- or N,N-disubstituted amino-alkyl, preferably N-mono- or N,N-disubstituted amino-C₁-C₇-alkyl, such as N-mono- or N,N-di-substituted aminomethyl, and azidoalkyl, preferably azido-C₁-C₇-alkyl, such as azidomethyl, cyano or C₁-C₇-alkanoyl, especially CHO or from C₂-C₇-alkenyl.

In unsubstituted or substituted “heterocyclyl”, heterocyclyl is preferably a heterocyclic radical that is unsaturated (=carrying the highest possible number of conjugated double bonds in the ring(s); preferably a heteroaryl), saturated or partially saturated and is preferably a monocyclic or in a broader aspect of the invention bicyclic or tricyclic ring; and has 3 to 24, more preferably 4 to 16, most preferably 4 to 10 ring atoms; wherein one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; which heterocyclic radical (heterocyclyl) is unsubstituted or substituted by one or more, especially 1 to 3, substituents independently selected from the group consisting of the substituents defined above for substituted alkyl and oxo (═O); and where heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]-dioxol-5-yl and 2,3-dihydro-benzo[1,4]dioxin-6-yl, each of these radicals being unsubstituted or substituted by one or more, preferably up to three, substitutents selected from those mentioned above for substituted alkyl, from alkenyl, e.g. C₁-C₇-alkenyl, and from oxo, especially from the group consisting of lower alkyl, especially methyl or tert-butyl, lower alkoxy, especially methoxy, oxo and halo.

In unsubstituted or substituted “cycloalkyl”, cycloalkyl is preferably a saturated mono- or bicyclic hydrocarbon group with 3 to 16, more preferably 3 to 9 ring carbon atoms, especially C₃-C₈-cycloalkyl, e.g. cyclopropyl, cyclobutyl, cyclopentyl, cyclohexyl, cycloheptyl or cyclooctyl, and is substituted by one or more, preferably one to three, substitutents independently selected from those described for substituted alkyl, especially from C₁-C₇-alkyl and hydroxy, or is (preferably) unsubstituted.

In unsubstituted or substituted “alkanoyl” (or alkylcarbonyl), alkanoyl is preferably formyl or more preferably C₂-C₂₀— yet more preferably C₂-C₇-alkanoyl, such as acetyl, propanoyl or butyroyl, is linear or branched and is substituted with one or more, especially up to three, substitutents independently selected from those mentioned above for substituted alkyl or is preferably unsubstituted as mentioned above, or is formyl (—CHO). Correspondingly, in unsubstituted or substituted “aroyl”, aroyl is preferably aryl-carbonyl (aryl-C(═O)—) wherein aryl is defined as above, e.g. benzoyl or naphthoyl, and is unsubstituted or substituted by one or more, preferably up to three, substituents independently selected from those mentioned above for alkyl.

In “amino-alkyl” (also a special variant of substituted alkyl), alkyl is preferably as defined above and is unbranched or branched. The amino moiety is preferably bound to a terminal carbon atom. Preferred is amino-C₁-C₇-alkyl, especially aminomethyl.

In “N-mono- or N,N-disubstituted amino-alkyl”, alkyl is preferably as defined above and is unbranched or branched. The mono- or disubstituted amino moiety is preferably bound to a terminal carbon atom. The substituents are preferably selected from unsubstituted or substituted alkyl, especially C₁-C₇-alkyl or phenyl-C₁-C₇-alkyl, such as methyl, ethyl or benzyl, acyl, especially C₁-C₇-alkanoyl, such as acetyl, unsubstituted or substituted aryl, preferably as defined above, especially phenyl, unsubstituted or substituted aroyl, preferably as defined above, e.g. benzoyl, and unsubstituted or substituted cycloalkyl, preferably as defined above, especially cyclopropyl, cyclobutyl, cyclopentyl or cyclohexyl.

In “azido-alkyl” (also a special variant of substituted alkyl), alkyl is preferably as defined above and is unbranched or branched. The azido moiety is preferably bound to a terminal carbon atom. Preferred is azido-C₁-C₇-alkyl, especially azidomethyl.

“Etherified hydroxyl” is preferably unsubstituted or substituted (preferably C₁-C₇-) alkyloxy, wherein the substituents are preferably independently selected from those mentioned for substituted alkyl, preferably methoxy or 3-(2-trimethylsilyl-ethoxy-methoxy; or is unsubstituted or substituted aryloxy wherein unsubstituted or substituted aryl is as defined above; e.g. substituted or preferably unsubstituted phenyloxy or naphthyloxy, respectively.

“Esterified hydroxyl” is preferably acyloxy with acyl as defined below, more preferably C₁-C₇-alkanoyloxy, such as acetoxy, benzoyloxy, naphthoyloxy, C₁-C₇-alkansulfonyloxy (alkyl-S(O)₂—O—), or phenyl- or naphthylsulfonyloxy (phenyl-S(O)₂—O— or naphthyl-S(O)₂—O—) wherein phenyl is unsubstituted or substituted, e.g. by one or more, e.g. up to 3, C₁-C₇-alkyl moieties.

In a first aspect, the invention relates to a compound of the formula (I),

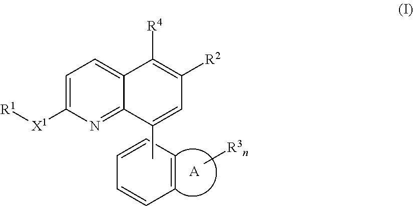

wherein
A represents an aryl, cycloalkyl, heterocyclyl condensed to the phenyl ring;
R¹ represents a substituent different from hydrogen;
R² represents hydrogen, halogen, nitro, optionally substituted amino, optionally substituted aryl, optionally substituted heterocyclyl;
R³ represents oxo (═O), amino, optionally substituted alkyl;
R⁴ represents hydrogen, alkoxy
X¹ represents a direct bond or an alkanediyl which is optionally interrupted by one or more groups selected from —O—, —C(O)—, —N(H)—, —N(lower alkyl)-, alkenediyl, provided that when more than one of said groups is present, two or more oxygen or nitrogen atoms are not bonded together (adjacent to each other);
n represent an integer from 0-3;
   except the compounds 2-methyl-8-naphthalen-quinoline and 2,2′-dimethyl-[8,8′]-biquinolinyl; or a salt thereof.

In preferred embodiments, which are preferred independently, collectively or in any combination or sub-combination, the invention relates to a compound of the formula (I), in free base form or in acid addition salt form, wherein the substituents are as defined herein.
A preferably represents—together with the phenyl ring to which it is attached—a moiety selected from the group consisting of naphthalene, 1,2,3,4-tetrahydronaphthalene, indole, isoindole, quinoline, isoquinoline, aryl, cycloalkyl, heterocyclyl condensed to the phenyl ring; said moiety optionally substituted by one or more substituents selected from the group consisting of lower alkyl, hydroxyl, oxo.
A in particular preferably represents—together with the phenyl ring to which is attached—a moiety selected from the group consisting of naphthalene, alpha or beta tetralone (in particular, 5-(3,4-dihydro-2H-naphthalen-1-one)), indole, oxindole, quinoline, (in particular, 5-quinoline), 2-(1H)quinolinone (=quinolinole) (in particular, 5-(1H)quinoline-2-one), isoquinoline (in particular, 8-isoquinoline), 1-(2H)isoquinolinone (=isoquinolinole).
A very particularly preferably represents—together with the phenyl ring to which is attached—a moiety selected from the group consisting of naphthalene, quinoline (in particular, 5-quinoline), isoquinoline (in particular, 8-isoquinoline).
R¹ advantageously represents a substituent different from hydrogen and different from unsubstituted alkyl.

R¹ preferably represents one of the following groups:

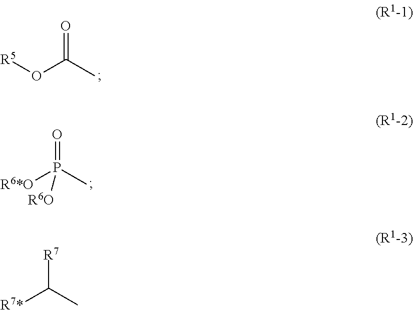

wherein
R⁵ represents hydrogen, unsubstituted alkyl, alkyl substituted by aryl;
R⁶ represents hydrogen, unsubstituted alkyl;
R⁶* represents hydrogen, unsubstituted alkyl;
R⁷ represents hydrogen, halogen, hydroxy, amino, N-substituted amino, N,N-di-substituted amino and
R⁷* represents hydrogen, carboxy, alkoxycarbonyl or
R⁷* and R⁷ represent together with the carbon to which they are attached an optionally substituted heterocycle.

Preferably, R⁷ and R⁷* are not at the same time both hydrogen.
R¹ particular preferably represents the group (R¹-1).
R¹ further preferably represents the group (R¹-2).
R² preferably represents hydrogen, chloro, bromo, iodo, nitro or
   amino, N-substituted amino, N,N-disubstituted amino, the substituents being selected from the group consisting of (C₁-C₄)-alkylcarbonyl, (C₁-C₄)-alkoxycarbonyl, aryl, heteroarylcarbonyl, benzoxycarbonyl, (C₁-C₄)-alkylsulfonyl or (C₁-C₆)-alkylcarbonyl wherein the alkyl of the (C₁-C₆)-alkylcarbonyl is substituted by NH₂, (C₁-C₄)-alkyl or (C₁-C₄)-alkoxycarbonyl or
   unsubstituted or substituted aryl the substituents being selected from the group consisting of halo, cyano, hydroxy, lower alkyl, lower haloalkyl, lower alkyl substituted by aryl, lower alkoxy, lower alkoxy substituted by aryl, lower alk-dioxy, lower alkanoyl, lower alkoxycarbonyl, tri(lower alkyl)silyl or
   unsubstituted or substituted heterocyclyl, said heterocyclyl being mono- or bicyclic with 5 to 10 ring atoms wherein one to four ring atoms are selected from the group consisting of nitrogen, oxygen and sulfur, said heterocyclyl preferably being a heteroaryl, said substituents being selected from the group consisting of halo, cyano, hydroxy, lower alkyl, lower haloalkyl, lower alkyl substituted by aryl, lower alkoxy, lower alkoxy substituted by aryl, lower alkanoyl, lower alkoxycarbonyl, tri(lower alkyl)silyl, oxo.
R² particular preferably represents hydrogen, iodo, chloro, nitro, or
   amino, N-substituted amino, N,N-disubstituted amino, the substituents being selected from the group consisting of ethoxycarbonyl, methylsulfonyl or
   unsubstituted or substituted phenyl the substituents being selected from the group consisting of hydroxy, methyl, ethyl, iso-propyl, tert.-butyl, trifluoromethyl, benzyl, methoxy, ethoxy, iso-propoxy, tert.-butoxy, benzoxy, acyl, methoxycarbonyl, ethoxycarbonyl, iso-propoxycarbonyl, tert.-butoxycarbonyl, trimethylsilyl or
   unsubstituted heterocyclyl or heterocyclyl substituted by one or two substitutents, said heterocyclyl being selected from the group consisting of pyrrole, pyridine, pyrimidine, indole, isoindole, furane, thiophene, 1,3-benzodioxole (in particular: thiophene such as 2- or 3-thiophene, pyridine such as pyridin-3-yl, pyrrole such as 2- or 3-pyrrole), said substituents being selected from the group consisting of hydroxy, methyl, ethyl, iso-propyl, tert.-butyl, trifluoromethyl, benzyl, methoxy, ethoxy, iso-propoxy, tert.-butoxy, benzoxy, acyl, methoxycarbonyl, ethoxycarbonyl, iso-propoxycarbonyl, tert.butoxycarbonyl, trimethylsilyl, oxo.
R³ preferably represents hydrogen (or alternatively expressed, when n=0), oxo, amino, lower alkyl, lower alkyl substituted by hydroxyl, lower alkanoyl, lower alkanoyloxy.
R³ particularly preferably represents oxo, amino, methyl, ethyl, propyl, n-butyl, sec-butyl, isobutyl, tert-butyl, 2,2-dimethylpropyl, 1,2,2-trimethylpropyl, 1-ethyl-propyl, substituted methyl, ethyl, propyl, n-butyl, sec-butyl, isobutyl, tert-butyl, 2,2-dimethylpropyl, 1,2,2-trimethylpropyl, 1-ethyl-propyl, the substituents being selected from the group consisting of hydroxy, lower acetyl, propanoyl, butyroyl, acetyloxy, propanoyloxy, butyroyloxy.
R⁴ preferably represents hydrogen, lower alkoxy (such as methoxy, ethoxy).
R⁴ particularly preferably represents hydrogen.
R⁵ preferably represents hydrogen, lower alkyl, lower alkyl substituted by phenyl (in particular R⁵ is: benzyl, methyl, ethyl, propyl, n-butyl, sec-butyl, isobutyl, tert-butyl, 2,2-dimethylpropyl, 1,2,2-trimethylpropyl, 1-ethyl-propyl).
R⁵ particularly preferably represents hydrogen, benzyl, methyl, ethyl.
R⁶ preferably represents hydrogen, lower alkyl (in particular: methyl, ethyl, propyl, n-butyl, sec-butyl, isobutyl, tert-butyl, 2,2-dimethylpropyl, 1,2,2-trimethylpropyl, 1-ethyl-propyl).
R⁶* particularly preferably represents hydrogen, methyl, ethyl.
R⁷ preferably represents hydrogen, halogen, hydroxy, amino, N-substituted amino, N,N-di-substituted amino, the substituents being selected from the group consisting of (C₁-C₄)-alkoxycarbonyl, benzoxycarbonyl, aminosulfonyl, (C₁-C₄)-alkoxycarbonylaminosulfonyl, benzoxycarbonyl-aminosulfonyl, and R⁷*preferably represents hydrogen, carboxy, (C₁-C₄)-alkoxycarbonyl or
R⁷* and R⁷ preferably represent together with the carbon to which they are attached a heterocycle optionally substituted by one or two oxo groups.
R⁷ particular preferably represents hydrogen, halogen, hydroxy, amino, N-substituted amino, N,N-disubstituted amino the substituents being selected from the group consisting of methoxycarbonyl, ethoxycarbonyl, tert-butoxycarbonyl, benzoxycarbonyl, aminosulfonyl, methoxycarbonyl-aminosulfonyl, ethoxycarbonyl-aminosulfonyl, benzoxycarbonyl-aminosulfonyl and R⁷* particular preferably represents carboxy, methoxycarbonyl, ethoxycarbonyl, tert-butoxycarbonyl, benzoxycarbonyl or
R⁷* and R⁷ particular preferably represent together with the carbon to which they are attached a five-membered heterocycle optionally substituted by one or two oxo groups, for example, R⁷* and R⁷ preferably represent together with the carbon to which they are attached 2H-tetrazol-5-yl, 2H-tetrazol-3-yl, 2-methyl-2H-tetrazol-5-yl, 2-methyl-2H-tetrazol-3-yl.
X¹ preferably represents a direct bond or a straight-chain or branched-chain C₁₋₁₂ alkandiyl which is optionally interrupted by one or more groups selected from —O—, —C(O)—, —N(H)—, —N(lower alkyl)-, or a straight-chain or branched-chain C₂₋₆ alkendiyl.
X¹ particular preferably represents a direct bond or —CH═CH— (vinyl) (cis- or trans-) or an alkandiyl selected from the group consisting of methandiyl, 1,2-ethanediyl, 1,3-propandiyl, said alkandiyl is optionally interrupted by one or more groups selected from —C(O)—, —N(H)—.
X¹ very particular preferably represents a direct bond, vinyl, methandiyl, 1,2-ethanediyl.
n preferably represents 0 or 1, most preferably 0.

In a further preferred embodiment, the invention relates to a compound of formula (I-A)

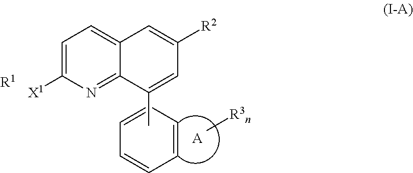

wherein the substituents are as defined herein.

In a further preferred embodiment, the invention relates to a compound of formula (I-B)

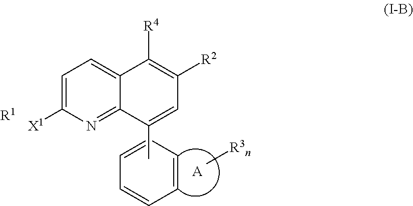

wherein the substituents are as defined herein and R⁴ does not represent hydrogen.

In a further preferred embodiment, the invention relates to a compound of formula (I-C)

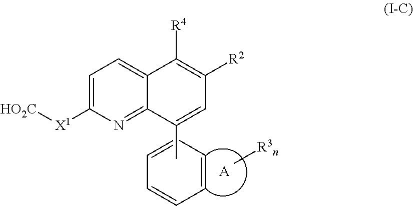

wherein the substituents are as defined herein.

In a further preferred embodiment, the invention relates to a compound of formula (I-D

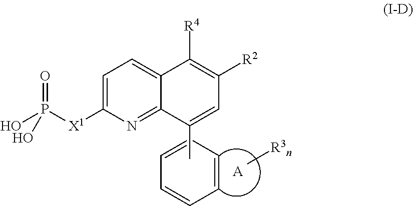

wherein the substituents are as defined herein.

In a further preferred embodiment, the invention relates to a compound of formula (I-E)

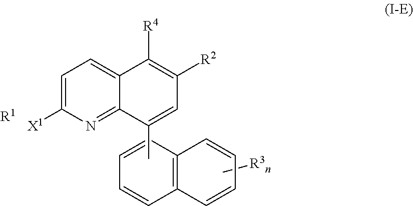

wherein the substituents are as defined herein.

The invention further relates to pharmaceutically acceptable prodrugs of a compound of formula (I). The invention thus relates also to pharmaceutically acceptable esters of a compound of formula (I); in particular to lower alkyl esters of a compound of formula (I).

The invention further relates to pharmaceutically acceptable metabolites of a compound of formula (I). The invention relates especially to the compounds of the formula (I) given in the Examples, as well as the methods of manufacture described therein. The compounds of formula (I) have valuable pharmacological properties, as described hereinbefore and hereinafter. Other preferred embodiments are mentioned above and below or in the claims which are incorporated by reference herein.

In a second aspect, the invention relates to a compound of formula (I′)

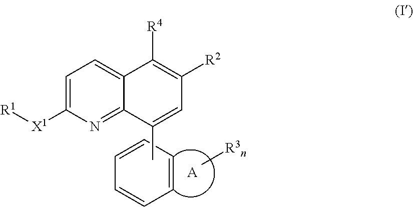

wherein
A represents an aryl, cycloalkyl, heterocyclyl condensed to the phenyl ring;
R¹ represents a substituent different from hydrogen;
R² represents hydrogen, halogen, nitro, optionally substituted amino, optionally substituted aryl, optionally substituted heterocyclyl;
R³ represents oxo (═O), amino, optionally substituted alkyl;
R⁴ represents hydrogen, alkoxy
X¹ represents a direct bond or an alkanediyl which is optionally interrupted by one or more groups selected from —O—, —C(O)—, —N(H)—, —N(lower alkyl)-, alkenediyl;
n represent an integer from 0-3;
   or a pharmaceutically acceptable salt thereof, or a pharmaceutically acceptable ester thereof, as medicament.

In preferred embodiments, the invention relates to a compound of formula (I′) wherein the substituents are as defined for a compound of formula (I), or a pharmaceutically acceptable salt thereof, or a pharmaceutically acceptable ester thereof, as medicament.

In a further aspect, the invention relates to methods of manufacturing a compound of formula (I) and intermediates thereof. A compound of the formula (I) may be prepared by processes that, though not applied hitherto for the new compounds of the present invention where they thus form new processes, are known per se.

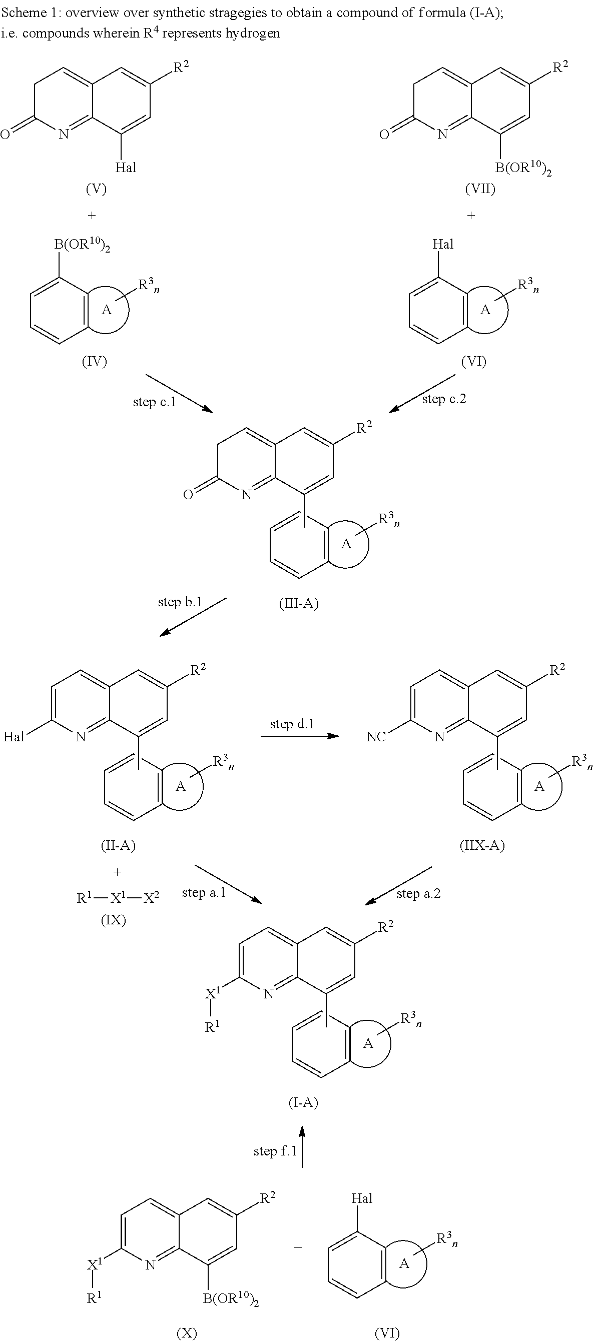

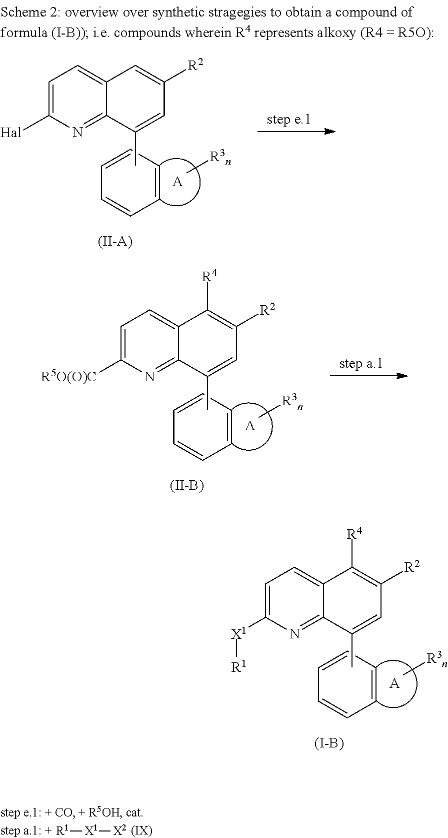

Schemes 1 and 2 illustrate methods for preparations according to this invention. Thus, the invention relates to a method of manufacturing (a manufacturing process) for a compound of formula (I) comprising the step of

method A: reacting a compound of formula (II)

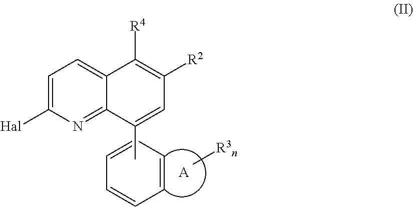

wherein the substituents are as defined herein and Hal represents halogen, in particular chloro, with a compound of formula (IX)

R¹—X¹—X² (IX)

wherein the substituents are as defined herein and X² represents either hydrogen or a leaving group; or
method B: converting a compound of formula (IIX)

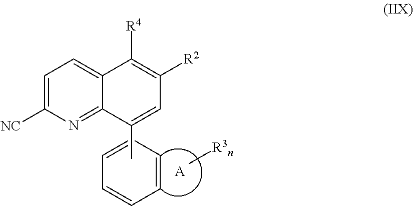

wherein the substituent are as defined herein into a compound of formula (I); or
method C: reacting a compound of formula (X)

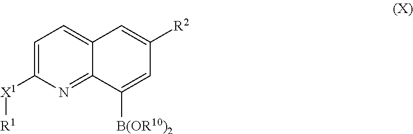

wherein the substituents are as defined herein and —B(OR¹⁰)₂ represents boronic acid or an ester thereof with a compound of formula (VI)

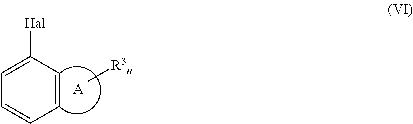

wherein the substituents are as defined herein and Hal represents halogen, in particular bromo, in the presence of an activating agent, such as a catalyst, in particular a homogeneous Pd catalyst; and, if desired, converting a compound of the formula (I) obtained according to method A, method B or method C into a different compound of the formula (I), and/or converting an obtainable salt of a compound of the formula (I) into a different salt thereof, and/or converting an obtainable free compound of the formula (I) into a salt thereof, and/or converting an obtainable ester of a compound of the formula (I) into the free acid thereof, and/or separating an obtainable isomer of a compound of the formula (I) from one or more different obtainable isomers of the formula (I).

Thus, the invention relates further to a method of manufacturing (a manufacturing process) a compound of formula (I-B) comprising

step e1: reacting a compound of formula (II-A)

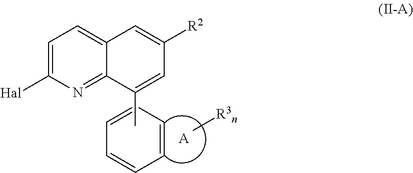

wherein the substituents are as defined herein and Hal represents halogen, in particular chloro, with an alcohol R⁵OH, wherein R⁵ represents unsubstituted alkyl, in the presence of CO and a homogeneous catalyst, such as a Pd catalyst, to obtain a compound of formula (II-B)

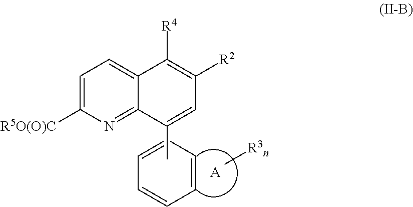

wherein the substituents are as defined herein and
step a.1: converting a compound of formula (II-B), optionally after purification, into a compound of formula (I-B)

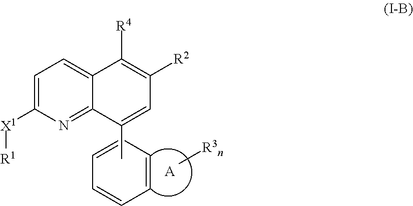

wherein the substituents are as defined herein, and, if desired,
converting a compound of the formula (I) obtained into a different compound of the formula (I), and/or converting an obtainable salt of a compound of the formula (I) into a different salt thereof, and/or converting an obtainable free compound of the formula (I) into a salt thereof, and/or converting an obtainable ester of a compound of the formula (I) into the free acid thereof, and/or separating an obtainable isomer of a compound of the formula (I) from one or more different obtainable isomers of the formula (I).

Step e.1 may take place at elevated CO pressure, e.g. 1-100 bar, preferably 5-50 bar.

### Reaction Conditions

Where temperatures are given hereinbefore or hereinafter, “about” has to be added, as minor deviations from the numeric values given, e.g. variations of ±10%, are tolerable. All reactions may take place in the presence of one or more diluents and/or solvents. The starting materials may be used in equimolar amounts; alternatively, a compound may be used in excess, e.g. to function as a solvent or to shift equilibrium or to generally accelerate reaction rates. Reaction aids, such as acids, bases or catalysts may be added in suitable amounts, as known in the field, required by a reaction and in line with generally known procedures.

### Protecting Groups

If one or more other functional groups, for example carboxy, hydroxy, amino or the like are or need to be protected in a starting material of the formula II or any precursor, because they should not take part in the reaction or disturb the reaction, these are such groups as are usually used in the synthesis of peptide compounds, and also of cephalosporins and penicillins, as well as nucleic acid derivatives and sugars. Protecting groups are such groups that are no longer present in the final compounds once they are removed, while groups that remain as substitutents are not protecting groups in the sense used here which is groups that are added at a starting material or intermediate stage and removed to obtain a final compound. For example, tert-butoxy if remaining in a compound of the formula (I) is a substituent, while if it is removed to obtain the final compound of the formula (I) it is a protecting group.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned above and below.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

### Further Optional Reactions and Conversions

A compound of the formula (I) may be converted into a different compound of the formula (I), e.g. by the methods as described herein, in particular below.

### Reduction

Carbonyl, hydroxy group reduction reactions are generally known. Typical condition suitable for the process as described herein are: carbonyl group reduction with sodium borohydride, e.g. as described in “Sodium Borohydride” in Encyclopedia of Reagents for Organic Synthesis (Ed: L. Paquette) 2004, J. Wiley & Sons, New York. Reduction of benzyl alcohols using triethylsilane in presence of TFA, eg as described in Tetrahedron Letters, 1993, 34, 1605-1608; or using a combination of hypophosphorous acid and iodine, e.g. as described in Tetrahedron Letters, 2001, 42, 831-833, the content of these documents is incorporated by reference.

Further, in a compound of the formula (I) wherein a substituent is present which is carboxy, said carboxy can be reduced to hydroxymethyl, e.g. by treatment first with ethylchloroformate in the presence of a tertiary nitrogen base, such as triethylamine or diisopropylethylamine, in an appropriate solvent, e.g. a cyclic ether, such as tetrahydrofuran, preferably at temperatures in the range from −50° C. to 30° C., followed by treatment with a reducing agent, e.g. sodium borohydride, in an appropriate solvent or solvent mixture, such as an alcohol, e.g. methanol, preferably at a temperature in the range from −50 to 20° C., e.g. from −20 to 10° C.

### Buchwald Reaction

This reaction, also known as Buchwald amination or Buchwald-Hartwig reaction is generally known in the field. This reaction is catalyzed by transition metals, in particular Cu or Pd complexes or salts; takes place in the presence of one or more basic compounds (such as an amine or an alkalialkoxide) and one or more diluents (such as polar aprotic diluents). Further details may be found in the examples.

### Fluorination

Methods to convert carbonyl and hydroxy groups into the corresponding fluoro compounds are generally known. Typical conditions suitable for the process are described e.g. in J. Org. Chem., 1986, 51, 3508-3513 or J. Am. Chem. Soc. 1984, 106, 4189-4192; the content of which is incorporated by reference.

### Alkylation

Carbonyl groups may be converted in the corresponding alkylated hydroxyl group using a Grignard reaction. Typical conditions suitable for the process are described, e.g. in Synthesis, 1981, 585-604. Further, carbonyl groups may be converted in the corresponding dialkylated compounds using a multi-step protocol, e.g. as described in Chem. Ber., 1985, 118, 1050-1057. Furthermore, carbonyl groups may be converted in the corresponding ispiro cyclopropane compound in two steps by Wittig olefination, e.g. as described in Chem. Rev., 1989, 89, 863-927, and subsequent cyclopronation reaction, e.g. Simmons-Smith as described in Org. React., 2001, 58, 1-415; the content of the above documents is incorporated by reference.

### Suzuki-Coupling

Reaction conditions, starting materials and catalysts for a Suzuki(-Miyaura) reaction are generally known in the field. This reaction typically takes place by palladium-catalyzed crosscoupling of organoboranes (e.g. of formula (IV) or (VII)) or a reactive derivative thereof, with a halogen derivative (e.g. of the formula (V) or (VI)). The reaction may be performed in analogy to the procedure described by K. Jones, M. Keenan, and F. Hibbert [Synlett, 1996, (6), 509-510].

Thus, the invention further relates to a process for manufacturing a compound of formula (III) which comprises method a) reacting a compound of formula (V)

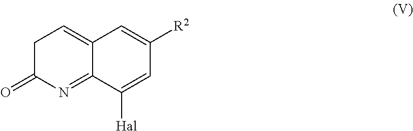

wherein the substituents are as defined above and hal represents halogen, in particular bromo, with a compound of formula (IV)

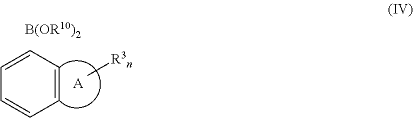

wherein the substituents are as defined above and —B(OR¹⁰)₂ represents boronic acid or an ester thereof, or
method b) reacting a compound of formula (VI)

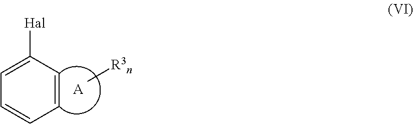

wherein the substituents are as defined above and hal represents halogen, in particular bromo, with a compound of formula (VII)

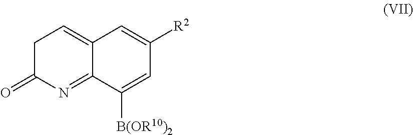

wherein the substituents are as defined above and —B(OR¹⁰)₂ represents boronic acid or an ester thereof; in the presence of an activating agent, such as a catalyst, in particular a homogeneous Pd catalyst; and optionally converting a substituent R² or R³ into an other substituent R² or R³.

Further, Suzuki coupling may be useful to convert a compound of formula (I), (II), (III), (V), (VII) wherein R² represents halo, in particular iodo, into another compound of formula (I), (II), (III), (V), (VII) wherein R² represents optionally substituted aryl.

Further, Suzuki coupling may be useful to convert a compound of formula (X) into a compound of formula (I).

### Halogenation

Reaction conditions, starting materials and catalysts for converting lactames to halogen compounds are generally known in the field. This reaction typically takes place in the presence of a halogenating agent, in particular P(O)Hal₃., such as POCl3.

Thus, the invention further relates to a process for manufacturing a compound of formula (II) which comprises reacting a compound of formula (III)

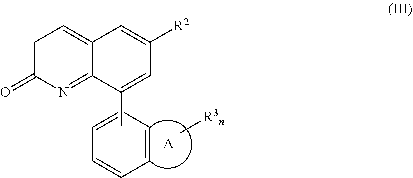

wherein the substituents are as defined above with a halogenating agent, optionally in the presence of a diluent and optionally in the presence of an reaction aid, to obtain the corresponding compound of formula (II)

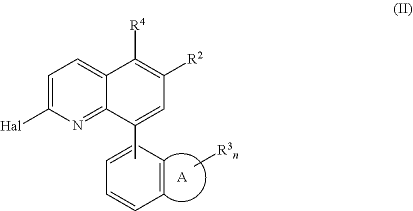

wherein the substituents are as defined above and Hal represents halogen to obtain said compound of formula (II).

Also in the optional process steps, carried out “if desired”, functional groups of the starting compounds which should not take part in the reaction may be present in unprotected form or may be protected for example by one or more of the protecting groups mentioned hereinabove under “protecting groups”. The protecting groups are then wholly or partly removed according to one of the methods described there.

Salts of a compound of formula (I) with a salt-forming group may be prepared in a manner known per se. Acid addition salts of compounds of formula (I) may thus be obtained by treatment with an acid or with a suitable anion exchange reagent. A salt with two acid molecules (for example a dihalogenide of a compound of formula I) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula I. Salts can usually be converted to free compounds, e.g. by treating with suitable basic compounds, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula (I) itself. Enantiomers may be separated through the formation of diastereomeric salts, for example by salt formation with an enantiomer-pure chiral acid, or by means of chromatography, for example by HPLC, using chromatographic substrates with chiral ligands.

It should be emphasized that reactions analogous to the conversions mentioned in this chapter may also take place at the level of appropriate intermediates (and are thus useful in the preparation of corresponding starting materials).

### Starting Materials

The starting materials of the formulae (II), (III), (IV), (V), (VI), (VII), (IIX), (IX), (X) as well as other starting materials (including intermediates) mentioned herein, e.g. below, can be prepared according to or in analogy to methods that are known in the art, are known in the art and/or are commercially available. Novel starting materials, in particular compound of the formula (II), (III) and (IIX), as well as processes for the preparation thereof, are likewise an embodiment of the present invention. In the preferred embodiments, such starting materials are used and the reactions chosen are selected so as to enable the preferred compounds to be obtained.

A compound of formula (X) may be obtained by converting the corresponding hydroxy compound (XI)

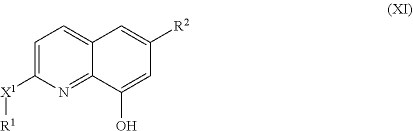

e.g. via the triflate, into the boronic acid or boronic acid ester, optionally in the presence of a diluent and optionally in the presence of a reaction aid.

In the synthesis of starting materials, the symbols (R¹, R², R³, n and so on) in the formulae given in the starting materials and intermediates given below have the meanings given for a compound of the formula (I) or as indicated specifically.

In a further aspect, the invention relates to the use of compounds of formula (I) as defined herein. As indicated above, compounds of formula (I) and (I′) are FPPS inhibitors and are thus useful as medicaments.

In further embodiments, the invention relates also

to a compound of formula (I) or (I′) for use in the treatment of a warm-blooded animal, especially a human, preferably for the treatment of an FPPS dependent disorder;
to the use of a compound of the formula (I), or a pharmaceutically acceptable salt thereof, in the treatment of an FPPS dependent disease;
to the use of a compound of the formula (I), or a pharmaceutically acceptable salt thereof, for the manufacture of a pharmaceutical preparation useful in the treatment of an FPPS dependent disease;
to a method of treatment comprising administering a compound of the formula (I), or a pharmaceutically acceptable salt thereof, in a therapeutically effective amount to a warm-blooded animal, especially a human, especially where in need of such treatment;
to a pharmaceutical preparation for the treatment of an FPPS-dependent disease, comprising a compound of the formula (I), or a pharmaceutically acceptable salt thereof, and a pharmaceutically acceptable carrier;
to a method of preparing such a pharmaceutical preparation, comprising mixing a compound of the formula (I), or a pharmaceutically acceptable salt thereof, with at least one pharmaceutically acceptable carrier material.

The activity of the compounds of the present invention as FPPS inhibitors can be tested using the scintillation proximity principal similar to a previously reported fatty acid synthase assay using a phospholipid-coated flashplate (see Weiss D R, Glickman J F (2003) Characterization of Fatty Acid Synthase Activity Using Scintillation Proximity. Assay and Drug Development Technologies; 1 (1-2):161-6). Prior FPPS assay methods have used organic:aqueous extraction to separate substrate from product. These methods are extremely time consuming and not compatible with testing large numbers (greater than 20,000) compounds. The FlashPlate method described below has the advantages of enabling the rapid testing of large numbers of compounds, easily, and directly. The product formation can be detected by using a phospholipid-coated “Flashplate” (trademark, PerkinElmer Lifesciences) which comprises surface-embedded scintillation materials. The lipophilic tritiated FPP which is formed binds to the plate while the tritiated IPP does not. The radiolabelled lipophilic product of the reaction is thus captured on the “Image FlashPlate” which emits photons when tritium is in close proximity. Additionally the use of the LEADseeker imager General Electric, Amersham Lifesciences Division, Cardiff, GB is incorporated which has distinct advantages in plate reading time and in reduced compound interference from yellow compounds over the previously cited Fatty Acid synthase assay. (Weiss Glickman 2003). Results of assayed compounds are given below.

Due to their ability to inhibit FPPS, and thus on the one hand cholesterol biosynthesis, on the other hand protein farnesylation, the compounds of the formula (I) are, inter alia, useful in the treatment or in the manufacture of pharmaceutical preparations for the treatment of cholesterol biosynthesis related disorders, e.g. for the lowering of the cholesterol level in blood, on the one hand, and/or protein farnesylation related disorders on the other hand, especially proliferative diseases such as cancer or tumor diseases. Metastasis, especially also bone metastasis, of any cancer or tumor disease is to be included especially. A compound of the formula (I) may also be used to diminish the susceptibility to cholera toxin by diminishing the number of membrane bound Gₛ protein molecules and for the treatment of pertussis toxin induced coughing by diminishing the number of G proteins. All these disorders are referred to as FPPS-dependent diseases hereinafter (the plural also including the singular, i.e. only one disease).

Where subsequently or above the term “use” is mentioned (as verb or noun) (relating to the use of a compound of the formula (I) or a pharmaceutically acceptable salt thereof and comparable embodiments of the invention like methods of their use and the like), this includes any one or more of the following embodiments of the invention, respectively: the use in the treatment of an FPPS-dependent disease, the use for the manufacture of pharmaceutical compositions for use in the treatment of an FPPS-dependent disease, methods of use of one or more compounds of the formula (I) in the treatment of an FPPS-dependent disease, the use of, the use of pharmaceutical preparations comprising one or more compounds of the formula (I) for the treatment of an FPPS-dependent disease, a process for the manufacture of a pharmaceutical preparation for the treatment of an FPPS-dependent disease, preferably also comprising making it ready for use in such treatment (e.g. adding an instruction insert (e.g. package leaflet or the like), formulation, appropriate preparation, adaptation for specific uses, customizing and the like), and the use of a compound of the formula (I) for such preparation, and/or all other prophylactic or therapeutic uses mentioned hereinbefore or below, a method of treatment comprising administering a compound of the formula (I) for the treatment of an FPPS-dependent disease and one or more compounds of the formula (I) for use in the treatment of a protein kinase dependent disease, as appropriate and expedient and if not stated otherwise. In particular, diseases to be treated and are thus preferred for “use” of a compound of formula (I) are selected from FPPS-dependent disease (“dependent” meaning dependent “on the activity of”, but also “supported”, not only “solely dependent”, e.g. in case where the FPPS activity is inadequate absolutely or in a given physiological context, either directly or indirectly due to other (e.g. preceding) regulatory mechanisms) diseases mentioned herein, especially proliferative diseases mentioned herein.

Based on the property of the compounds of formula (I) as potent FPPS inhibitors, the compounds of formula (I) are especially suitable for the treatment of neoplastic diseases such as cancers and tumors (especially solid tumours but also leukemias, benign or especially malignant tumors), e.g. carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, a neoplasia, a neoplasia of epithelial character or lymphomas, as well as myeloma, especially multiple myeloma, myelodysplastic syndrome, AML (acute myeloid leukemia), AMM (angiogenic myeloid metaplasia), mesothelioma, glioma and glioblastoma, or bone cancer.

On the other hand, compounds of the formula (I) are especially appropriate for treating cholesterol biosynthesis related disorders, e.g. for the lowering of the cholesterol level in blood, for example for the treatment (including prophylaxis) of atherosclerosis, bilestones, especially cholelithiasis, lipocalcinogranulomatosis, hypercholesterolaemia, hyperlipoproteinaemia, cholesterol crystal embolism, myocardial infection, cerebral infarction, angina pectoris, and/or the like, also as auxiliary treatment together with other treatment (including prophylactic) measures.

Furthermore, in view of the activities disclosed herein, the compounds of the formula (I) are especially appropriate for treating in general or inflammation related types of bone loss, including osteoporose, arthritis including rheumatoid arthritis, osteoarthritis and Paget's disease.

The invention relates also to pharmaceutical compositions comprising a compound of formula (I), to their use in the therapeutic (in a broader aspect of the invention also prophylactic) treatment or a method of treatment of an FPPS-dependent disease, especially the preferred diseases mentioned above, to the compounds for said use and to pharmaceutical preparations and their manufacture, especially for said uses, and to methods of use of a compound of the formula (I) in the treatment of such a disease.

The present invention also relates to pro-drugs of a compound of formula (I), in particular an ester that converts in vivo to the compound of formula (I) as such. Any reference to a compound of formula (I) is therefore to be understood as referring also to the corresponding pro-drugs of the compound of formula (I), as appropriate and expedient.

The pharmacologically acceptable compounds of the present invention may be present in or employed, for example, for the preparation of pharmaceutical compositions that comprise an effective amount of a compound of the formula (I), or a pharmaceutically acceptable salt thereof, as active ingredient together or in admixture with one or more inorganic or organic, solid or liquid, pharmaceutically acceptable carriers (carrier materials).

The invention relates also to a method of treatment for a disease that responds to inhibition of an FPPS-dependent disease and/or a proliferative disease, which comprises administering a prophylactically or especially therapeutically (against the mentioned diseases) effective amount of a compound of formula (I) according to the invention, or a tautomer thereof or a pharmaceutically acceptable salt thereof, especially to a warm-blooded animal, for example a human, that, on account of one of the mentioned diseases, requires such treatment.

Furthermore, the invention provides the use of a compound according to the definitions herein, or a pharmaceutically acceptable salt, or a hydrate or solvate thereof for the preparation of a medicament for the treatment of an FPPS-dependent disease, especially a proliferative disease or a cholesterol biosynthesis related disorder.

The invention especially relates to the use of a compound of the formula (I) (or a pharmaceutical formulation comprising a compound of the formula I) in the treatment of one or more of the diseases mentioned above and below where the disease(s) respond or responds (in a beneficial way, e.g. by partial or complete removal of one or more of its symptoms up to complete cure or remission) to an inhibition of FPPS, especially where FPPS shows (in the context of other regulatory mechanisms) inadequately high or more preferably higher than normal (e.g. constitutive) activity.

In a further aspect, the invention relates to a combination of a compound of formula (I) with one or more other therapeutically active agents. Thus, a compound of formula (I) can be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents.

A compound of formula (I) can besides or in addition be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Thus, a compound of the formula (I) may also be used in combination with other anti-proliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibit tors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; N-bisphosphonic acid derivatives; cathepsin K inhibitors; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array PioPharma, AZD6244 from AstraZeneca, PD181461 from Pfizer, leucovorin, EDG binders, antileukemia compounds, ribonucleotide reductase inhibitors, S-adenosylmethionine decarboxylase inhibitors, antiproliferative antibodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in antiproliferative treatment, combination with anti-inflammatory drugs is included.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, colchicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
j) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a PI3K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr <1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TN P-470.

Compounds which target, decrease or inhibit the activity of a protein or lipid phosphatase are e.g. inhibitors of phosphatase 1, phosphatase 2A, or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “N-bisphosphonic acid derivatives” as used herein includes, but is not limited to, 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529. especially etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA. All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The term “cathepsin K inhibitors” as used herein includes, but is not limited to, the compounds exemplified in U.S. Pat. No. 6,353,017B1 and WO 03/020278A1.

The term “mTOR inhibitors” relates to compounds which inhibit the mammalian target of rapamycin (mTOR) and which possess antiproliferative activity such as sirolimus (Rapamune®), everolimus (Certican™, CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity. For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2″-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Somatostatin receptor antagonists as used herein refers to compounds which target, treat or inhibit the somatostatin receptor such as octreotide, and SOM230.

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium. Angiostatic steroids as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art, such as in the documents cited above.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The invention also provides a pharmaceutical preparation, comprising a compound of formula (I) as defined herein, or an N-oxide or a tautomer thereof, or a pharmaceutically acceptable salt of such a compound, or a hydrate or solvate thereof, and at least one pharmaceutically acceptable carrier.

A compound of formula (I) can be administered alone or in combination with one or more other therapeutic compounds, possible combination therapy taking the form of fixed combinations or the administration of a compound of the invention and one or more other therapeutic (including prophylactic) compounds being staggered or given independently of one another, or the combined administration of fixed combinations and one or more other therapeutic compounds. A compound of formula (I) can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

The dosage of the active ingredient (=compound of the formula (I) in free and/or pharmaceutically acceptable salt form) depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

The dose of a compound of the formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 2.5 g per person per day, divided preferably into 1 to 3 single doses which may, for example, be of the same size. Usually, children receive half of the adult dose.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or, creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The invention relates also to pharmaceutical compositions comprising an effective amount, especially an amount effective in the treatment of one of the above-mentioned disorders, of a compound of formula (I) or an N-oxide or a tautomer thereof together with one or more pharmaceutically acceptable carriers that are suitable for topical, enteral, for example oral or rectal, or parenteral administration and that may be inorganic or organic, solid or liquid.

There can be used for oral administration especially tablets or gelatin capsules that comprise the active ingredient together with pharmaceutically acceptable carrier materials, e.g. diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilisers, wetting compounds and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectionning, dissolving or lyophilising processes, and comprise approximately from 1% to 99%, especially from approximately 1% to approximately 20%, active ingredient(s).

Additionally, the present invention provides a compound of formula (I), or a pharmaceutically acceptable salt of such a compound, for use in a method for the treatment of the human or animal body, especially for the treatment of a disease mentioned herein, most especially in a patient requiring such treatment.

The present invention also relates to the use of a compound of formula (I), or a pharmaceutically acceptable salt of such a compound, for the preparation of a medicament for the treatment of a proliferative disease.

Furthermore, the invention relates to a method for the treatment of a proliferative disease which responds to an inhibition of FPPS, which comprises administering a compound of formula (I) or a pharmaceutically acceptable salt thereof, wherein the radicals and symbols have the meanings as defined above, especially in a quantity effective against said disease, to a warm-blooded animal requiring such treatment.

Furthermore, the invention relates to a pharmaceutical composition for treatment of solid or liquid tumours in warm-blooded animals, including humans, comprising an antitumor effective dose of a compound of the formula (I) as described above or a pharmaceutically acceptable salt of such a compound together with a pharmaceutical carrier.

The following examples serve to illustrate the invention without limiting its scope. If not indicated otherwise, reactions are conducted at room temperature. Temperatures are given in degrees Celsius (° C.). Unless otherwise indicated, the reactions take place at room temperature under N₂-atmosphere. Where the term “heated at” is used, this means “heated to and kept at”. Ratios e.g. of solvents or eluents in mixtures and the like are given as volume by volume (v/v) ratios. The following abbreviations are used:

### Abbreviations:

Anal. elemental analysis (for indicated atoms, difference between calculated and measured value ≦0.4%)
aq. aqueous
brine saturated solution of NaCl in water
conc. concentrated
d day(s)
DIPE diisopropyl-ether
DMAP dimethylaminopyridine
DMF dimethyl formamide
DMSO dimethyl sulfoxide
Et ethyl
ether diethylether
Et₃N triethylamine
EtOAc ethyl acetate
EtOH ethanol
eq. equivalent
Ex. Example
h hour(s)
HPLC high pressure liquid chromatography: System: HPLC-system Acquity, Waters; Column: BEH C18 1.7 μM; Gradient: t_{Ret}: retention time [min]: Linear gradient: [CH₃CN (0.1% TFA)] and [H₂O (0.1% TFA)], 2→100% CH₃CN (0.1% TFA) in 1.6 min+0.4 min 100% CH₃CN (0.1% TFA); flow rate 1 ml/min; detection at 215 nm.
Hyflo Hyflo Super Cel® (filtering aid based on diatomaceous earth; obtainable from Fluka, Buchs, Switzerland)
HOAc acetic acid
HV high vacuum
l litre(s)
Me methyl
MeOH methanol
min minute(s)
m.p. melting point
MPLC medium pressure liquid chromatography
   Combi Flash system: Systeme: Combi Flash Companion from Isco, Inc.; Columns: RediSep® flash column, Teledyne Isco, filled with 4 g, 12 g, 40 g or 120 g of SiO₂; application to column: either mixture is dissolved as a concentrated solution in eluent, or a solution of the mixture is concentrated together with SiO₂ in vacuo and applied as powder)
   Reversed phase chromatography: Gilson system: reversed phase Nucleosil C18 (H₂O/CH₃CN+TFA), generally product obtained as TFA-salt by concentration and lyophilisation, or as free base after neutralization with NaHCO₃, partial concentration and filtration or extraction with EtOAc
MS mass spectrum
NMP N-methyl-pyrrolidone
Ph phenyl
propylphosphonic anhydride: 2,4,6-tripropyl-1,3,5,2,4,6-trioxatriphosphorinane-2,4,6-trioxide [68957-94-8]; 50% in DMF
R_{f} ratio of fronts (TLC)
rt room temperature
sat. saturated
THF tetrahydrofuran (distilled from Na/benzophenone)
TFA trifluoroacetic acid
TLC thin layer chromatography: The R_{f} values which indicate the ratio of the distance moved by each substance to the distance moved by the eluent front are determined on silica gel thin-layer plates (Merck, Darmstadt, Germany) by thin-layer chromatography using the respective named solvent systems.
t_{Ret} retention time (HPLC)

### EXAMPLE 1

### 8-Naphthalen-1-quinoline-2-carboxylic acid

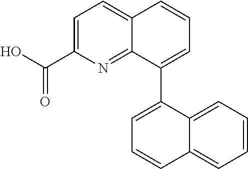

To 210 mg Pd/C 10% in 20 ml MeOH, 8-naphthalen-1-yl-quinoline-2-carboxylic acid benzyl ester (210 mg, 0.54 mMol) is added, followed by ammonium formiate (170 mg, 2.7 mMol). This mixture is stirred for 40 min at 65° C. Then the catalyst is filtered off and extensively washed with MeOH. The filtrate is concentrated and triturated in hexane. The crude product is dryed (HV; 70° C.), yielding the title compound: MS: [M+1]⁺=300; ¹H-NMR (DMSO-d₆): δ ppm 8.50 (d, 1H), 8.14 (d, 1H), 8.02 (d, 2H), 7.94 (d, 1H), 7.75 (m, 2H), 7.64 (t, 1H), 7.50 (m, 2H), 7.32 (t, 1H), 7.27 (d, 1H).

The starting material is prepared as follows:

### Step 1.1: 8-Hydroxy-quinoline-2-carboxylic acid benzyl ester

8-Hydroxy-quinoline-2-carboxylic acid (5.67 g, 30.0 mMol), PPh₃ (11.8 g, 45 mMol) and benzylalcohol (2.96 ml, 28.5 mMol) are dissolved in 500 ml THF and cooled in an ice bath. Then diethyl azodicarboxylate (7.0 ml, 45 mMol) is added dropwise during 5 min and the mixture is stirred for 30 min. The reaction mixture is concentrated in vacuo, the residue diluted with water and EtOAc, the aq. phase separated off and extracted twice with EtOAc. The organic layers are washed with water and brine, dried (Na₂SO₄) and concentrated. Trituration from ether and filtration gives from the concentrated filtrate the title compound after column chromatography (SiO₂; hexane/EtOAc 17:3→4:1): MS: [M+1]⁺=280; TLC(EtOAc): Rf=0.59.

### Step 1.2: 8-Trifluoromethanesulfonyloxy-quinoline-2-carboxylic acid benzyl ester

8-Hydroxy-quinoline-2-carboxylic acid benzyl ester (1.396 g, 5.0 mMol) and pyridine (1.61 ml, 20 mMol) are dissolved in 75 ml CH₂Cl₂/dioxane 2:1 and cooled to −75° C. Then a solution of (F₃CSO₂)₂O (1.65 ml, 10 mMol) in 1 ml CH₂Cl₂ is added and the mixture is allowed to warm up slowly to 5° C. during 260 min. The reaction mixture is diluted with water, sat. NaHCO₃ and EtOAc, the aq. phase separated off and extracted twice with EtOAc. The organic layers are washed with water and brine, dried (Na₂SO₄) and concentrated, yielding the title compound: MS: [M+1]⁺=412; ¹H-NMR (DMSO-d₆): δ ppm 8.80 (d, 1H), 8.33 (d, 1 H), 8.27 (d, 1H), 8.07 (d, 1H), 7.89 (t, 1H), 7.58 (d, 2H), 7.43 (m, 3H), 5.50 (s, 1H₂C).

### Step 1.3: 8-(4,4,5,5-Tetramethyl-[1,3,2]dioxaborolan-2-yl)-quinoline-2-carboxylic acid benzyl ester

8-Trifluoromethanesulfonyloxy-quinoline-2-carboxylic acid benzyl ester (1.5 g, 3.64 mMol) is dissolved in 20 ml DMF. Then bis-(pinacolato)-diboron (1.1 g, 4.3 mMol), potassium acetate (1.07 g, 10.9 mMol) and 6 g molecular sieves 4 Å are added. After degassing the mixture, [1,1′-bis(diphenylphosphino)ferrocene]palladium(II) chloride, complex with CH₂Cl₂, (90 mg, 0.11 mMol) is added. The reaction mixture is stirred for 1% h at 80° C., diluted with brine and EtOAc, the aq. phase separated off and extracted twice with EtOAc. The organic layers are washed with water and brine, dried (Na₂SO₄) and concentrated, yielding the title compound, which is used in the next step without further purification.

### Step 1.4: 8-Naphthalen-1-yl-quinoline-2-carboxylic acid benzyl ester

1.0 mMol 8-(4,4,5,5-Tetramethyl-[1,3,2]dioxaborolan-2-yl)-quinoline-2-carboxylic acid benzyl ester is dissolved in 5 ml toluene. Then 1-bromo-naphthaline (140 μl, 1.00 mMol) and K₂CO₃ (207 mg, 1.5 mMol) are added. After degassing the mixture, (Ph₃P)₄Pd (50 mg, 0.043 mMol) is added. The reaction mixture is stirred for 18 h at 90° C., diluted with water and EtOAc, the aq. phase separated off and extracted twice with EtOAc. The organic layers are washed with water and brine, dried (Na₂SO₄) and concentrated. Chromatography (Combi Flash; hexane→+hexane/EtOAc 17:3) gives the title compound: MS: [M+1]⁺=390; TLC(EtOAc/hexane 1:1): Rf=0.70.

### EXAMPLE 2

the following derivatives are obtained analogously to Ex. 1:

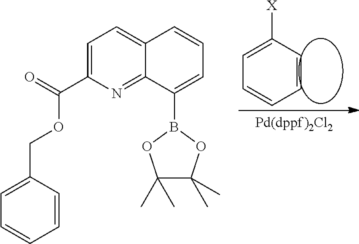

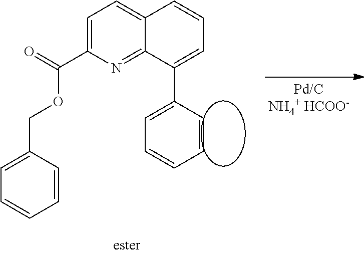

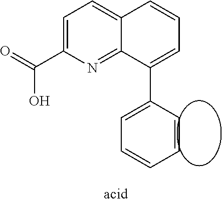

| Ex. 2. | 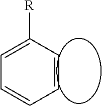 | TLC R_{f} | MS [M + 1]⁺ | m.p. [° C.] |
|---|---|---|---|---|
| a.i ester a.ii acid | 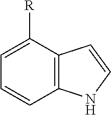 | 0.50¹⁾ 0.45²⁾ | 379 289 | |
| b.i ester b.ii acid | 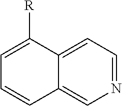 | 0.47³⁾ 0.21²⁾ | 391 301 | 148-149 |
| c.i ester c.ii acid | 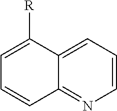 | 0.53³⁾ 0.27²⁾ | 391 301 | 189-191 |
| d.i ester d.ii acid | 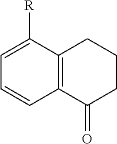 | 0.50¹⁾ 0.53²⁾ | 408 318 | |
| e.i ester e.ii acid | 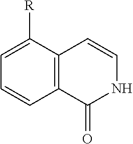 | 0.40³⁾ 0.20²⁾ | 407 317 | |
| f.i ester f.ii acid | 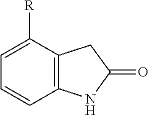 | 0.57³⁾ 0.13²⁾ | 395 305 | |
| g.i ester g.ii acid | 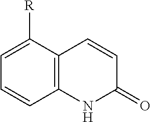 | 0.13²⁾ | 407 317 | |

¹⁾hexane/EtOAc 1:1;
²⁾CH₂Cl₂/MeOH 5:1;
³⁾EtOAc;

### EXAMPLE 3

### {[(8-Naphthalen-1-yl-quinoline-2-carbonyl)-amino]-methyl}-phosphonic acid

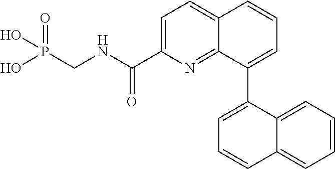

A solution of {[(8-naphthalen-1-yl-quinoline-2-carbonyl)amino]-methyl}-phosphonic acid diethyl ester (104 mg, 0.23 mMol) in 5 ml CH₂Cl₂ is cooled in an icebath. Then bromo-trimethyl-silane (268 μl, 2.07 mMol) is added and the mixture is stirred for 18.5 h at rt. The mixture is concentrated in vacuo, the residue re-dissolved in MeOH and again concentrated. Trituration in EtOAc and filtration yields the title compound as the hydrobromide salt (C₂₁H₁₇N₂O₄P.HBr): MS: [M+1]⁺=393; ¹H-NMR (DMSO-d₆): δ ppm 8.70 (d, 1H), 8.21 (d, 1H), 8.17 (d, 1H), 8.00 (d, 2H), 7.88 (m, 2H), 7.63 (t, 1H), 7.57 (d, 1H), 7.49 (t, 1H), 7.4 (m, 2H), 7.35 (t, 1H), 3.55 (m, 1 H), 3.18 (m, 1H).

The starting material is prepared as follows:

### Step 3.1: {[(8-Naphthalen-1-yl-quinoline-2-carbonyl)-amino]-methyl}-phosphonic acid diethyl ester

To an ice cooled solution of 8-naphthalen-1-yl-quinoline-2-carboxylic acid (119 mg, 0.40 mMol) and aminomethyl-phosphonic acid diethyl ester (134 mg, 0.80 mMol) in 2.5 ml DMF, Et₃N (560 μl, 4 mMol), DMAP (12 mg) and propylphosphonic anhydride (456 μl, 0.80 mMol) are added. The mixture is stirred for 2.5 h at rt and then poured into brine and EtOAc. The aq. phase is separated off and extracted with EtOAc. The organic layers are washed with water and brine, dried (Na₂SO₄) and concentrated. Chromatography (SiO₂; hexane/EtOAc 1:1→1:3→EtOAc) gives the title compound as an oil: MS: [M+1]⁺=449; TLC (EtOAc): R_{f}=0.25.

### EXAMPLE 4

### (8-Naphthalen-1-yl-quinolin-2-yl)-phosphonic acid

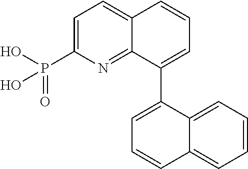

A solution of (8-naphthalen-1-yl-quinolin-2-yl)-phosphonic acid diethyl ester (100 mg, 0.255 mMol) in 10 ml CH₂Cl₂ is cooled in an icebath. Then bromo-trimethyl-silane (330 μl, 2.55 mMol) is added and the yellowish solution is stirred for 24 h at rt. The mixture is concentrated in vacuo, the residue re-dissolved in MeOH and again concentrated (two times). Trituration from tert-butylmethylether/CH₂Cl₂ yields the title compound as the hydrobromide salt (C₁₉H₁₄NO₃P.HBr): MS: [M+1]⁺=336; ¹H-NMR (DMSO-d₆): δ ppm 8.56 (dd, 1H), 8.14 (d, 1H), 7.99 (d, 2H), 7.92 (dd, 1H), 7.80 (t, 1H), 7.75 (d, 1 H), 7.62 (t, 1H), 7.49 (m, 2H), 7.32 (d, 2H).

The starting material is prepared as follows:

### Step 4.1: 8-Naphthalen-1-yl-1H-quinolin-2-one

A mixture of 8-bromo-2(1H)-quinolinone [1.98 g, 8.84 mMol (synthesis see: Eur. J. Org. Chem. 2003, 1559)] and 1-naphthaleneboronic acid (1.84 g, 10.7 mMol) in 19 ml K₂CO₃ (1 M in H₂O) and 9 ml DMF is degassed by 3 times evacuation and flushing with N₂. Then Pd(PPh₃)₂Cl₂ (380 mg, 0.53 mMol) is added and the mixture is heated to 120° C. for 60 min. After filtration, the filtrate is diluted with water and EtOAc, the aq. phase separated off and extracted twice with EtOAc. The organic layers are washed with water and brine, dried (Na₂SO₄) and partially concentrated. The resulting suspension is filtered, the title compound washed with ice cold EtOAc and dried in HV at 40° C.: m.p.: 198-199° C.; MS: [M+1]⁺=272. More product can be isolated from the filtrate by chromatography (Combi Flash; CH₂Cl₂/acetone 99:1→92:8).

### Step 4.2: 2-Chloro-8-naphthalen-1-yl-quinoline

To 8-naphthalen-1-yl-1H-quinolin-2-one (1.66 g, 6.12 mMol), tetraethylammonium chloride (2.23 g, 13.5 mMol) and N,N-dimethylaniline (1.71 ml, 13.5 mMol) in 175 ml acetonitrile, POCl₃ (7.3 ml, 79.7 mMol) is added. This mixture is stirred for 80 min at 55° C. and then poured into 900 g ice. Vigorously stirring, warming up to rt, filtration, washing with water and drying gives the title compound: m.p.: 133-135° C.; MS: [M+1]⁺=290.

### Step 4.3: (8-Naphthalen-1-yl-Quinolin-2-yl)-phosphonic acid diethyl ester

A mixture of 2-chloro-8-naphthalen-1-yl-quinoline (200 mg, 0.69 mMol), (EtO)₃P (460 mg, 2.76 mMol) and dry NiCl₂ (30 mg, 0.23 mMol) is stirred for 5 h at 170° C. After cooling to it dilution with CH₂Cl₂ and addition of 4 g SiO₂, the mixture is concentrated. The resulting powder is put on top of a SiO₂-column and the title compound chromatographed with CH₂Cl₂/EtOAc 19:1→4:1): m.p.: 128° C.; MS: [M+1]⁺=392.

### EXAMPLE 5

### [(8-Naphthalen-1-yl-quinolin-2-ylamino)-methyl]-phosphonic acid

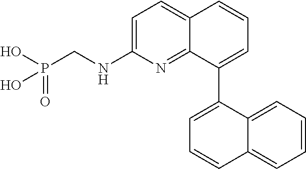

To a solution of [(8-naphthalen-1-yl-quinolin-2-ylamino)-methyl]-phosphonic acid diethyl ester (70 mg, 0.16 mMol) in 5 ml CH₂Cl₂, bromo-trimethyl-silane (220 μl, 1.7 mMol) is given.

To drive the reaction to completion, another portion of 220 μl bromo-trimethyl-silane is added after 23 h, followed by a 110 μl portion after 47 h. After 71 h at rt, the mixture is concentrated in vacuo, the residue re-dissolved in CH₂Cl₂ and again concentrated. Trituration from ether yields the title compound as the hydrobromide salt: Anal. (+1 HBr+2 H₂O+1 ether): C, H, N, Br; MS: [M+1]⁺=365.

The starting material is prepared as follows:

### Step 5.1: 8-Bromo-6-nitro-1H-quinolin-2-one

To an ice cooled solution of 8-bromo-1H-quinolin-2-one [190.0 g, 0.848 Mol (synthesis see: Eur. J. Org. Chem. 2003, 1559)] in 836 ml TFA, 304 ml of fuming HNO₃ is added via an addition funnel over 90 min and the temperature is kept between 0 and 5° C. The dark reaction mixture is stirred for 7 h at rt and then poured into 2 kg of ice/water. Vigorously stirring, warming up to rt, filtration, washing with water, sat. NaHCO₃ and again water with subsequent drying gives the title compound: m.p.: decomp. >250° C.; MS: [M+1]⁺=249.

### Step 5.2: 8-Naphthalen-1-yl-6-nitro-1H-quinolin-2-one

8-Bromo-6-nitro-1H-quinolin-2-one (207.8 g, 0.772 Mol) in 1.6 l of DMF is degassed by evacuation (3 times) and flushing with N₂. The yellow suspension is heated to 80° C. (internal temperature) and 1-naphthaleneboronic acid (149.3 g, 0.868 Mol) and 1.53 l K₂CO₃ (1 M in H₂O) is added. Then Pd(PPh₃)₂Cl₂ (29.77 g, 0.042 Mol) is added and the dark brown mixture is heated to 92° C. (internal temperature) for 3 h. After cooling to rt and filtration over highflow, the filter cake was washed with 10 l of hot CH₂Cl₂. The aq. phase is separated off and extracted with 5 l of CH₂Cl₂. The organic layers are combined and partially concentrated. The resulting suspension is filtered, washed with hexane and dried under reduced pressure to obtain the title compound: m.p.: 234-235° C.; MS: [M+1]⁺=317. More product can be isolated from the filtrate by chromatography (3 kg SiO₂; CH₂Cl₂/acetone 99:1→92:8).

### Step 5.3: 2-Chloro-8-naphthalen-1-yl-6-nitro-quinoline

To 8-naphthalen-1-yl-6-nitro-1H-quinolin-2-one (63.7 g, 0.201 Mol), tetraethylammonium chloride (72 g, 0.402 Mol) and N,N-dimethylaniline (54 ml, 0.243 Mol) in 1.8 l acetonitrile, POCl₃ (180 ml, 1.96 Mol) is added. This mixture is stirred over night at reflux and then cooled to 45° C. The warm mixture is carefully added to 2.5 l of warm water (45° C.). Vigorously stirring, cooling to rt, filtration, washing with water and drying gives the crude compound which is purified by chromatography (2 kg SiO₂; CH₂Cl₂/hexane 1:1) to obtain the title compound: m.p.: 224.5-225° C.; MS: [M+1]⁺=335.

### Step 5.4: [(6-Nitro-8-naphthalen-1-yl-quinolin-2-ylamino)-methyl]-phosphonic acid diethyl ester

2-Chloro-8-naphthalen-1-yl-6-nitro-quinoline (3.0 g, 9.0 mMol) and aminomethyl-phosphonic acid diethyl ester (9.0 g, 54 mMol) are dissolved in 40 ml NMP. After addition of Cs₂CO₃ (3.2 g, 9.9 mMol) and a trace of KI, the mixture is stirred vigorously for 3 h at 80° C. The suspension is poured into brine and extracted three times with EtOAc. The organic layers are washed with water and brine, dried (Na₂SO₄) and concentrated. Chromatography (SiO₂; hexane/EtOAc 1:1→EtOAc→EtOAc/MeOH 9:1) and trituration from hexane gives the title compound: m.p.: 187-188° C.; MS: [M+1]⁺=466; TLC (EtOAc): R_{f}=0.20.

### Step 5.5: [(6-Amino-8-naphthalen-1-yl-quinolin-2-ylamino)-methyl]-phosphonic acid diethyl ester

Hydrogenation of [(6-nitro-8-naphthalen-1-yl-quinolin-2-ylamino)-methyl]-phosphonic acid diethyl ester (1.78 g, 3.8 mMol) in 100 ml MeOH/THF 4:1 in presence of 0.6 g Raney Nickel, filtration, extensive washing of the catalyst with MeOH and concentration of the filtrate gives the title compound: MS: [M+1]⁺=436; TLC (CH₂Cl₂/MeOH 9:1): R_{f}=0.53.

### Step 5.6: [(6-Iodo-8-naphthalen-1-yl-quinolin-2-ylamino)-methyl]-phosphonic acid diethyl ester

To [(6-amino-8-naphthalen-1-yl-quinolin-2-ylamino)-methyl]-phosphonic acid diethyl ester (980 mg, 2.25 mMol) in 11 ml conc. HCl, pieces of ice are added and the mixture is cooled to −18° C. Then a solution of NaNO₂ (311 mg, 4.5 mMol) in 20 ml H₂O is added during 10 min and it is stirred for 20 min. The reddish solution is added dropwise to a solution of KI (20.3 g, 122 mMol) in 65 ml H₂O. The resulting suspension is warmed up to rt and stirred for 5 h, giving a brown solution which is diluted with ether and water. The aq. phase is separated off and extracted twice with ether. The organic layers are washed with 2 N NaOH, diluted Na₂S₂O₃ solution, water and brine, dried (Na₂SO₄) and concentrated, yielding the title compound: MS: [M+1]⁺=547; TLC (EtOAc): R_{f}=0.27.

### Step 5.7: [(8-Naphthalen-1-yl-quinolin-2-ylamino)-methyl]-phosphonic acid diethyl ester

A mixture of [(6-iodo-8-naphthalen-1-yl-quinolin-2-ylamino)-methyl]-phosphonic acid diethyl ester (137 mg, 0.25 mMol), Et₃N (38 μl, 0.27 mMol), 25 mg Pd/C (10%; Engelhard 4505) and 15 ml MeOH is hydrogenated. The catalyst is filtered off, washed with MeOH and the filtrate concentrated. The residue is dissolved in water and EtOAc, the aq. phase separated off and extracted twice with EtOAc. The organic layers are washed with water and brine, dried (Na₂SO₄) and concentrated, yielding the title compound: MS: [M+1]⁺=421; TLC (EtOAc): R_{f}=0.29.

### EXAMPLE 6

### [(6-Nitro-8-naphthalen-1-yl-quinolin-2-ylamino)-methyl]-phosphonic acid

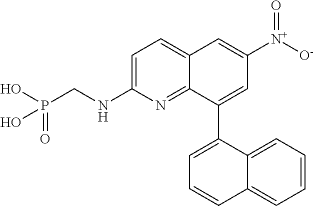

The title compound is obtained analogousely to Ex. 5 starting from [(6-nitro-8-naphthalen-1-yl-quinolin-2-ylamino)methyl]-phosphonic acid diethyl ester (93 mg, 0.20 mMol; Step 5.4) as the hydrobromide salt: Anal. (+1.3 HBr+2 H₂O): C, H, N, Br; MS: [M+1]⁺=410.

### EXAMPLE 7

### [(6-Amino-8-naphthalen-1-yl-quinolin-2-ylamino)-methyl]-phosphonic acid

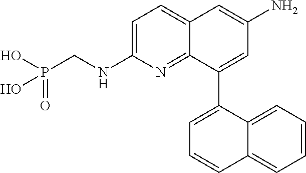

The title compound is obtained analogousely to Ex. 5 starting from [(6-amino-8-naphthalen-1-yl-quinolin-2-ylamino)-methyl]-phosphonic acid diethyl ester (87 mg, 0.20 mMol; Step 5.5) as the dihydrobromide salt: Anal. (+1.85 HBr+2 H₂O): C, H, N, Br; MS: [M+1]⁺=380; ³¹P-NMR (DMSO-d₆): δ16.7 ppm.

### EXAMPLE 8

### [(6-Iodo-8-naphthalen-1-yl-quinolin-2-ylamino)-methyl]-phosphonic acid

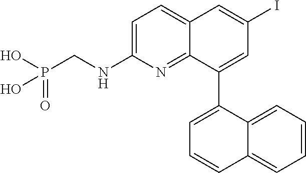

The title compound is obtained analogousely to Ex. 5 staring from [(6-iodo-8-naphthalen-1-yl-quinolin-2-ylamino)-methyl]-phosphonic acid diethyl ester (82 mg, 0.15 mMol; Step 5.6) as the hydrobromide salt: MS: [M+1]⁺=491.

### EXAMPLE 9

### {[8-Naphthalen-1-yl-6-(1H-pyrrol-3-yl)-quinolin-2-ylamino]-methyl}-phosphonic acid

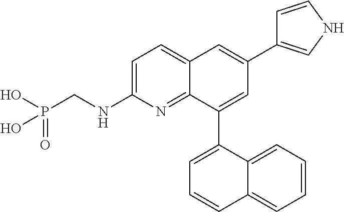

The title compound is obtained analogousely to Ex. 5 starting from {[8-naphthalen-1-yl-6-(1-triisopropylsilanyl-1H-pyrrol-3-yl)-quinolin-2-ylamino]-methyl}-phosphonic acid diethyl ester (110 mg, 0.17 mMol) as the hydrobromide salt: MS: [M+1]⁺=430.

The starting material is prepared as follows:

### Step 9.1: {[8-Naphthalen-1-yl-6-(1-triisopropylsilanyl-1H-pyrrol-3-yl)-quinolin-2-ylamino]-methyl}-phosphonic acid diethyl ester

[(6-Iodo-8-naphthalen-1-yl-quinolin-2-ylamino)-methyl]-phosphonic acid diethyl ester (162 mg, 0.30 mMol; Step 5.6) is dissolved in 1.5 ml degassed DMF. Then 1-(triisopropylsilanyl)-1H-pyrrole-3-boronic acid (120 mg, 0.45 mMol), 0.48 ml 2 M Na₂CO₃ in H₂O and [1,1-bis(diphenylphosphino)ferrocene]dichloropalladium(II) dichloromethane complex (21 mg, 0.025 mMol) are added. The mixture is stirred for 2% h at 80° C., then poured into brine and extracted three times with EtOAc. The organic layers are washed with water and brine, dried (Na₂SO₄) and concentrated. Chromatography (Combi Flash; hexane→hexane/EtOAc 3:2→EtOAc) gives the title compound: MS: [M+1]⁺=642; TLC (EtOAc): R_{f}=0.31.

### EXAMPLE 10

### [(8-Naphthalen-1-yl-6-pyridin-3-yl-quinolin-2-ylamino)-methyl]-phosphonic acid

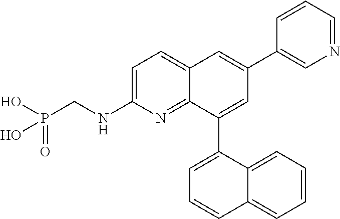

The title compound is obtained analogousely to Ex. 4 starting from [(8-naphthalen-1-yl-6-pyridin-3-yl-quinolin-2-ylamino)-methyl]-phosphonic acid diethyl ester (83 mg, 0.167 mMol) as the dihydrobromide salt: Anal. (+2 HBr+2 H₂O): C, H, N, Br; MS: [M+1]⁺=442.

The starting material is prepared as follows:

### Step 10.1: [(8-Naphthalen-1-yl-6-pyridin-3-yl-quinolin-2-ylamino)-methyl]-phosphonic acid diethyl ester

[(6-Iodo-8-naphthalen-1-yl-quinolin-2-ylamino)-methyl]-phosphonic acid diethyl ester (136 mg, 0.25 mMol; Step 5.6) is dissolved in 1.25 ml degassed DMF. Then pyridine-3-boronic acid (46 mg, 0.37 mMol), 0.40 ml 2 M Na₂CO₃ in H₂O and [1,1′-bis(diphenylphosphino)ferrocene]dichloropalladium(II) dichloromethane complex (19 mg, 0.022 mMol) are added. The mixture is stirred for 3 h at 80° C. and then worked up analogously to Ex. 9.1 giving the title compound: MS: [M+1]⁺=498; TLC (CH₂Cl₂/MeOH 9:1): R_{f}=0.60.

### EXAMPLE 11

### (8-Naphthalen-1-yl-quinolin-2-ylmethyl)-phosphonic acid

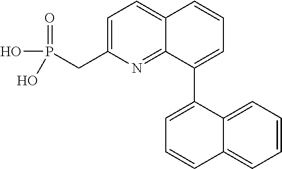

The title compound is obtained analogousely to Ex. 4 starting from (8-naphthalen-1-yl-quinolin-2-ylmethyl)-phosphonic acid diethyl ester (122 mg, 0.30 mMol) as its hydrobromide salt: Anal. (+0.85 HBr+2 H₂O+0.3 ether): C, H, N, Br; MS: [M+1]⁺=350; ³¹P-NMR (DMSO-d₆): δ 16.6 ppm.

The starting material is prepared as follows:

### Step 11.1: 8-Hydroxy-quinoline-2-carboxylic acid methyl ester

Me₃SiCl (3.8 ml, 30 mMol) is added to a suspension of 8-hydroxy-quinoline-2-carboxylic acid (2.0 g, 10.5 mMol) in 30 ml MeOH. Stirring for 16 at 50° C. gives a yellowish solution, which is poured into 250 ml water and 50 ml sat. NaHCO₃. The precipitated title compound is filtered off, washed with water and dried: MS: [M+1]⁺=204.

### Step 11.2: 8-Trifluoromethanesulfonyloxy-quinoline-2-carboxylic acid methyl ester

A solution of 8-hydroxy-quinoline-2-carboxylic acid methyl ester (2.03 g, 10 mMol) in 100 ml CH₂Cl₂ is cooled to −78° C. Then Et₃N (4.3 ml, 31 mMol) is dropped in, followed by a solution of trifluoromethanesulfonic anhydride (2.3 ml, 14 mMol) in 10 ml CH₂Cl₂. After 3 h at −78° C., the mixture is poured into EtOAc and water/sat. NaHCO₃ 10:1. The aq. phase is separated off and extracted twice with EtOAc. The organic layers are washed with water and brine, dried (Na₂SO₄) and concentrated. Crystallization from DIPE/hexane gives the title compound: m.p.: 77-78° C.; MS: [M+1]⁺=336.

### Step 11.3: 8-Naphthalen-1-yl-quinoline-2-carboxylic acid methyl ester

A solution of 8-trifluoromethanesulfonyloxy-quinoline-2-carboxylic acid methyl ester (1.05 g, 3.13 mMol) in 50 ml tert-butanol is degassed by repeated evacuation to HV and flushing with N₂. Then 1-naphthalene-boronic acid (0.59 g, 3.44 mMol), Pd(OAc)₂ (56 mg, 0.25 mMol), K₃PO₄ (1.592 g, 7.5 mMol) and 2-dicyclohexylphosphino-2′,4′,6′-triisopropyl-1,1′-biphenyl (179 mg, 0.375 mMol) are added successively. This mixture is stirred for 3 h at 80° C., cooled to rt and diluted with EtOAc and water. The aq. phase is separated off and extracted twice with EtOAc. The organic layers are washed with water and brine, dried (Na₂SO₄) and concentrated. Column chromatography (SiO₂; hexane/CH₂Cl₂1:1→1:2→CH₂Cl₂) and crystallization from hexane gives the title compound: m.p.: 157° C.; MS: [M+1]⁺=314; TLC(hexane/CH₂Cl₂ 2:3): R_{f}=0.11.

### Step 11.4: 2-Hydroxymethyl-8-naphthalen-1-yl-quinoline

A suspension of 8-naphthalen-1-yl-quinoline-2-carboxylic acid methyl ester (1.01 g, 3.22 mMol) and NaBH₄ (365 mg, 9.6 mMol) in 50 ml tert-butanol is stirred for 3 h at 40° C. and 1 h at 60° C. After addition of 25 ml H₂O to the cooled mixture, it is concentrated partially in vacuo. The residue is dissolved in EtOAc and water, the aq. phase separated off and extracted twice with EtOAc. The organic layers are washed with water and brine, dried (Na₂SO₄) and concentrated. Column chromatography (SiO₂; CH₂Cl₂/hexane 1:1→CH₂Cl₂) gives the title compound: MS: [M+1]⁺=286; TLC(CH₂Cl₂): R_{f}=0.17.

### Step 11.5: 2-Chloromethyl-8-naphthalen-1-yl-quinoline hydrochloride

To an ice cooled solution of 2-hydroxymethyl-8-naphthalen-1-yl-quinoline (216 mg, 0.76 mMol) in 5 ml acetonitrile, SOCl₂ (0.25 ml, 3.4 mMol) is added. The solution is stirred for 1 h and then concentrated in vacuo, giving the title compound: MS: [M+1]⁺=304/306.

### Step 11.6: (8-Naphthalen-1-yl-quinolin-2-ylmethyl)-phosphonic acid diethyl ester

A mixture of 2-chloromethyl-8-naphthalen-1-yl-quinoline hydrochloride (187 mg, 0.55 mMol) and (EtO)₃P (0.41 g, 2.4 mMol) is stirred for 2 h at 170° C. The cooled mixture is dissolved in EtOAc and H₂O/sat. NaHCO₃ 9:1, the aq. layer separated off and extracted twice with EtOAc. The organic phases are washed with water and brine, dried (Na₂SO₄) and concentrated. Chromatography (Combi Flash; hexane/EtOAc 3:2→EtOAc) gives the title compound: Anal. (+0.5; H₂O): C, H, N; MS: [M+1]⁺=406; ³¹P-NMR (DMSO-d₆): δ 24.4 ppm.

### EXAMPLE 12

### {[(8-Naphthalen-1-yl-quinolin-2-ylmethyl)-amino]-methyl}-phosphonic acid

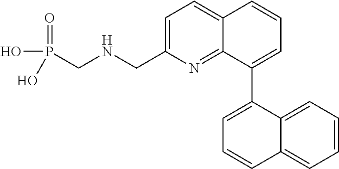

To an ice cooled solution of {[(8-naphthalen-1-yl-quinolin-2-ylmethyl)-amino]-methyl}-phosphonic acid ethyl ester (67 mg, 0.15 mMol) in 5 ml CH₂Cl₂, Me₃SiBr (194 μl, 1.5 mMol) is added. After 7 d at rt, the mixture is concentrated, re-dissolved in MeOH and again concentrated. Reversed phase chromatography, concentration and lyophilisation gives the title compound: Anal. (+1.1; H₂O): C, H, N; MS: [M+1]⁺=379; ¹H-NMR (CD₃OD): δ ppm 8.52 (d, 1H), 8.13 (d, 1H), 8.01 (m, 2H), 7.86 (d, 1H), 7.82 (t, 1H), 7.68 (t, 1H), 7.59 (m, 2H), 7.51 (t, 1H), 7.39 (d, 1 H), 7.35 (m, 1H), 4.54 (s, 2H), 2.79 (quint., 2H).

The starting material is prepared as follows:

### Step 12.1: {[(8-Naphthalen-1-yl-quinolin-2-ylmethyl)-amino]-methyl}-phosphonic acid ethyl ester

A mixture of 2-chloromethyl-8-naphthalen-1-yl-quinoline hydrochloride (169 mg, 0.50 mMol), aminomethyl-phosphonic acid diethyl ester (203 mg, 1.2 mMol), 22 mg KI and Cs₂CO₃ (293 mg, 0.90 mMol) in 10 ml tert-butanol is heated for 7 h at 100° C. This mixture is diluted with EtOAc and water, the aq. phase separated off and extracted twice with EtOAc. The organic layers are washed with water and brine, dried (Na₂SO₄) and concentrated. Chromatography [Combi Flash; CH₂Cl₂→EtOAc→EtOAc/(EtOAc+2% Et₃N) 4:1] and trituration from hexane gives the title compound: MS: [M+1]⁺=435; TLC(EtOAc): R_{f}=0.09; ³¹P-NMR (CDCl₃): δ 27.3 ppm.

### EXAMPLE 13

### rac. 2-[(tert-Butoxycarbonylamino-sulfonyl)-amino]-3-[(8-naphthalen-1-yl-quinoline-2-carbonyl)-amino]-propionic acid methyl ester

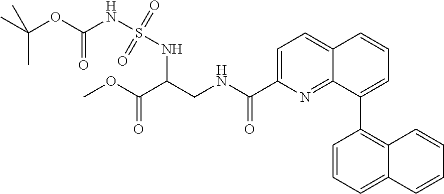

tert-Butyl (chlorosulfonyl)carbamate [prepared from chlorosulfonylisocyanate (166 μl, 1.91 mMol) and tert-butanol (305 μl, 3.25 mMol) in 13 ml CH₂Cl₂ as described in Heteroatom Chemistry 12, (2001), 1] is added dropwise to a suspension of rac. 2-amino-3-[(8-naphthalen-1-yl-quinoline-2-carbonyl)-amino]-propionic acid methyl ester hydrochloride (758 mg, 1.74 mMol) and Et₃N (532 μl, 3.82 mMol) in 9 ml CH₂Cl₂. After 90 min at rt, the mixture is diluted with CH₂Cl₂ and washed twice with 0.05 N HCl, water and brine. The aq. layers are re-extracted twice with CH₂Cl₂, the organic phases dried (Na₂SO₄) and concentrated. Chromatography (Combi Flash; hexane/EtOAc 4:1→1:1) gives the title compound: MS: [M+1]⁺=579; TLC(EtOAc/hexane 1:1): R_{f}=0.17.

The starting material is prepared as follows:

### Step 13.1: rac. 2-(Benzyloxycarbonyl-amino)-3-[(8-trifluoromethanesulfonyloxy-quinoline-2-carbonyl)-amino]-propionic acid methyl ester

A solution of 8-hydroxy-quinolin-2-carbonic acid (1.96 g, 10.4 mMol) in 100 ml CH₂Cl₂, 50 ml dioxane and Et₃N (8.6 ml, 62 mMol) is cooled to −70° C. Trifluoromethanesulfonic anhydride (3.6 ml, 21.8 mMol) dissolved in 15 ml CH₂Cl₂ is added dropwise. After 2 h at −70° C., another 0.35 ml trifluoromethanesulfonic anhydride are added and the mixture is slowly warmed up to 0° C. Then a suspension of rac. 3-amino-2-benzyloxycarbonylamino-propionic acid methyl ester (3.29 g, 11.4 mMol) in 60 ml CH₂Cl₂/dioxane 1:2 is added portion wise, the mixture warmed up to rt and stirred for 16 h. The reaction mixture is poured into 0.8 l EtOAc, 0.4 l sat. NaHCO₃ and 0.4 l H₂O, the aq. phase separated off and extracted twice with EtOAc. The organic layers are washed with water and brine, dried (Na₂SO₄) and concentrated. Column chromatography (SiO₂; CH₂Cl₂/EtOAc 19:1→9:1→88:12→85:15) gives the title compound: MS: [M+1]⁺=556; TLC(CH₂Cl₂/EtOAc 3:1): R_{f}=0.47.

### Step 13.2: rac. 2-(Benzyloxycarbonyl-amino)-3-[(8-naphthalen-1-yl-quinoline-2-carbonyl)amino]-propionic acid methyl ester

rac. 2-(Benzyloxycarbonyl-amino)-3-[(8-trifluoromethanesulfonyloxy-quinoline-2-carbonyl)amino]-propionic acid methyl ester (1.49 g, 2.68 mMol) in 150 ml tert-butanol is degassed by repeated evacuation to HV and flushing with N₂. Then 1-naphthalene-boronic acid (507 mg, 2.95 mMol), K₃PO₄ (1.37 g, 6.45 mMol), 2-dicyclohexylphosphino-2′,4′,6′-triisopropyl-1,1′-biphenyl (179 mg, 0.375 mMol) and Pd(OAc)₂ (54 mg, 0.24 mMol) are added successively. This mixture is stirred for 70 min at 80° C., cooled to rt and concentrated in vacuo. The residue is re-dissolved in EtOAc and water, the aq. phase separated off and extracted twice with EtOAc. The organic layers are washed with water and brine, dried (Na₂SO₄) and concentrated. Column chromatography (SiO₂; hexane/EtOAc 7:3→11:9) gives the title compound: MS: [M+1]⁺=534; TLC(hexane/EtOAc 1:1): R_{f}=0.20.

### Step 13.3: rac. 2-Amino-3-[(8-naphthalen-1-yl-quinoline-2-carbonyl)-amino]-propionic acid methyl ester hydrochloride

A mixture of rac. 2-(benzyloxycarbonyl-amino)-3-[(8-naphthalen-1-yl-quinoline-2-carbonyl)amino]-propionic acid methyl ester (925 mg, 1.73 mMol) and 140 mg Pd/C (10% Engelhard 4505) in 70 ml THF, 70 ml MeOH and 1.9 ml 1 M HCl is hydrogenated. Filtration and concentration of the filtrate gives the title compound: MS: [M-1-1]⁺=400.

### EXAMPLE 14

### rac. 2-[(Amino-sulfonyl)-amino]-3-[(8-naphthalen-1-yl-quinoline-2-carbonyl)amino]-propionic acid methyl ester

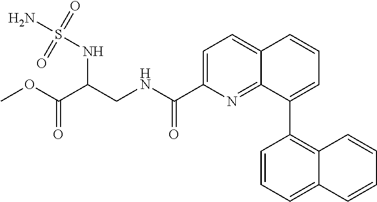

To a solution of rac. 2-[(tert-butoxycarbonylamino-sulfonyl)-amino]-3-[(8-naphthalen-1-yl-quinoline-2-carbonyl)-amino]-propionic acid methyl ester (265 mg, 0.458 mMol) in 5 ml dioxane, 5 ml HCl (4 M in dioxane) is added. After 110 min, the reaction mixture is concentrated in vacuo, giving the hydrochloride of the title compound.

Preparation of the free base: The reaction mixture is diluted with EtOAc and sat. NaHCO₃. The aq. layer is separated off and extracted twice with EtOAc. The organic phases are washed with water and brine, dried (Na₂SO₄) and concentrated. Reversed phase chromatography gives the title compound: MS: [M+1]⁺=479.

### EXAMPLE 15

### rac. 8-Naphthalen-1-yl-quinoline-2-carboxylic acid (1,1,4-trioxo-1lambda*6*-[1,2,5]thiadiazolidin-3-ylmethyl)-amide A and rac. 2-[(amino-sulfonyl)-amino]-3-[(8-naphthalen-1-yl-quinoline-2-carbonyl)-amino]-propionic acid B

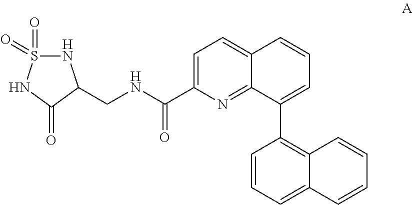

To rac. 2-[(amino-sulfonyl)-amino]-3-[(8-napthalen-1-yl-quinoline-2-carbonyl)-amino]-propionic acid methyl ester hydrochloride (0.24 mMol, Ex. 14) in 7 ml THF cooled in an ice bath, 1.5 ml 4 M aq. NaOH are added dropwise. After 20 min, the reaction mixture is poured into diluted citric acid and extracted 3 times with EtOAc. The organic phases are washed with water and brine, dried (Na₂SO₄) and concentrated. Chromatography (Combi Flash; EtOAc/EtOH 82:8→1:1) gives A followed by B. A: MS: [M+1]⁺=447. B: MS: [M+1]⁺=465.

### EXAMPLE 16

### [6-(3-Methoxy-phenyl)-8-naphthalen-1-yl-quinolin-2-yl]-phosphonic acid

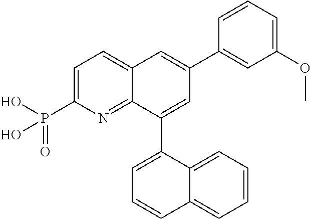

The title compound is obtained analogously to Ex. 4 starting from 6-(3-methoxy-phenyl)-8-naphthalen-1-yl-quinolin-2-yl]-phosphonic acid diethyl ester (84 mg, 0.169 mMol): MS: [M+1]⁺=442.

The starting material is prepared as follows:

### Step 16.1: 6-Amino-8-naphthalen-1-yl-1H-quinolin-2-one

8-Naphthalen-1-yl-6-nitro-1H-quinolin-2-one (1.43 g, 4.52 mMol; Step 5.2) in 80 ml MeOH and 20 ml THF is hydrogenated in presence of 0.7 g Raney-Nickel (in EtOH; B113 W Degussa). The catalyst is filtered off through Hyflo and extensively washed with MeOH/THF 4:1. Concentration of the filtrate and chromatography gives the title compound: MS: [M+1]⁺=287; TLC (CH₂Cl₂/aceton4 9:1): R_{f}=0.13.

### Step 16.2: 6-Iodo-8-naphthalen-1-yl-1H-quinolin-2-one

To 6-amino-8-naphthalen-1-yl-1H-quinolin-2-one (520 mg, 1.82 mMol) in 11.6 ml conc. HCl, pieces of ice are added and the mixture is cooled to −16° C. Then a solution of NaNO₂ (251 mg, 3.63 mMol) in 15 ml H₂O is added during 5 min and it is stirred for 20 min. The yellow solution is added dropwise to a solution of KI (16.3 g, 98 mMol) in 68 ml H₂O. The brown suspension is warmed up to rt and stirred for 5 h and then diluted with EtOAc and water. The aq. phase is separated off and extracted twice with EtOAc. The organic layers are washed with 2 N NaOH, 10% Na₂S₂O₃ solution and brine, dried (Na₂SO₄), concentrated and triturated in hexane, yielding the title compound: MS: [M+1]⁺=547; TLC (EtOAc): R_{f}=0.27.

### Step 16.3: 6-(3-Methoxy-phenyl)-8-naphthalen-1-yl-1H-quinolin-2-one

A suspension of 6-iodo-8-naphthalen-1-yl-1H-quinolin-2-one (460 mg, 1.16 mMol), 3-methyoxy-phenyl boronic acid (211 mg, 1.39 mMol) and 2.5 ml 1 M aq. K₂CO₃ in 5 ml DMF is degassed by repeated evacuation by HV and flushing with N₂. Then (Ph₃P)₂PdCl₂ (49 mg, 0.069 mMol) is added and heated up to 110° C. for 1 h. The cold mixture is poured into brine and extracted three times with EtOAc. The organic layers are washed with water and brine, dried (Na₂SO₄) and concentrated. Chromatography (Combi Flash; hexane→CH₂Cl₂/EtOAc 99:1→19:1) gives the title compound: MS: [M+1]⁺=378; TLC (CH₂Cl₂/EtOAc 19:1): R_{f}=0.14.

### Step 16.4: 2-Chloro-6-(3-methoxy-phenyl)-8-naphthalen-1-yl-quinoline

To a solution of 6-(3-methoxy-phenyl)-8-naphthalen-1-yl-1H-quinolin-2-one (480 mg, 1.27 mMol) in 20 ml acetonitrile, Et₄NCl (463 mg, 2.8 mMol), N,N-dimethylaniline (355 μl, 2.8 mMol) and POCl₃ (1.51 ml, 16.5 mMol) are added. After stirring for ½ h at 60° C., the cooled solution is poured into a mixture of EtOAc, ice water and sat. NaHCO₃, the aq. layer separated off and extracted twice with EtOAc. The organic phases are washed with water and brine, dried (Na₂SO₄) and concentrated. Chromatography (Combi Flash; hexane/(EtOAc/ether 1:1) 99:1→19:1) gives the title compound: MS: [M+1]⁺=396/398; HPLC: t_{Ret}=1.63.

### Step 16.5: 6-(3-Methoxy-phenyl)-8-naphthalen-1-yl-quinolin-2-yl]-phosphonic acid diethyl ester

2-Chloro-6-(3-methoxy-phenyl)-8-naphthalen-1-yl-quinoline (223 mg, 0.56 mMol) is dissolved in 3 ml degassed toluene. Then diethylphosphite (80 μl, 0.62 mMol), Et₃N (86 μl, 0.62 mMol) and (Ph₃P)₄Pd (65 mg, 0.056 mMol) are added. This solution is stirred for 21 h at 100° C. in a sealed vessel. The cold reaction mixture is diluted in water and EtOAc, the aq. layer separated off and extracted twice with EtOAc. The organic phases are washed with water and brine, dried (Na₂SO₄) and concentrated. Chromatography (Combi Flash; hexane/EtOAc 4:1→3:7) gives the title compound: MS: [M+1]⁺=498; HPLC: t_{Ret}=1.54; TLC(hexane/EtOAc 1:2): R_{f}=0.31.

### EXAMPLE 17

### (8-Naphthalen-1-yl-6-thiophen-2-yl-quinolin-2-yl)-phosphonic acid

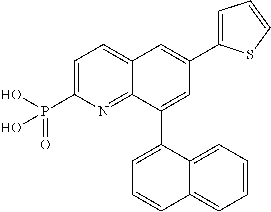

A solution of (8-naphthalen-1-yl-6-thiophen-2-yl-quinolin-2-yl)phosphonic acid diethyl ester (75 mg, 0.158 mMol) in 3 ml CH₂Cl₂ is cooled in an icebath. Then bromo-trimethyl-silane (102 μl, 0.79 mMol) is added and the orange solution is stirred for 5 h at rt in a sealed vessel. Addition of hexane leads to a precipitation, which can be filtered off and washed with hexane, giving the hydrobromide salt of the title compound: Anal. (+0.72 HBr+3.5; H₂O+0.2 hexane): C, H, N, S, Br; MS: [M+1]⁺=418; ¹H-NMR (DMSO-d₆): δ ppm 8.58 (dd, 1H), 8.40 (s, 1H), 8.96 (s, 1H), 8.02 (d, 1H), 8.00 (d, 1H), 7.90 (dd, 1H), 7.78 (d, 1H), 7.67 (d, 1H), 7.63 (t, 1H), 7.55 (d, 1H), 7.49 (t, 1H), 7.35 (m, 2H), 7.18 (dd, 1H).

The starting material is prepared as follows:

### Step 17.1: (8-Naphthalen-1-yl-6-nitro-quinolin-2-yl)-phosphonic acid diethyl ester

To a suspension of 2-chloro-8-naphthalen-1-yl-6-nitro-quinoline (5.62 g, 16.8 mMol; Step 5.3) in 50 ml degassed toluene, diethylphosphite (3.24 ml, 25.2 mMol), Et₃N (2.57 ml, 18.5 mMol) and (Ph₃P)₄Pd (1.94 g, 1.68 mMol) are added. This mixture is stirred for 2 h at 100° C. in a sealed vessel, cooled to rt and diluted with 0.5 l EtOAc and 0.5 l 0.1N aq. HCl. The aq. layer is separated off and extracted twice with EtOAc. The organic phases are washed with water and brine, dried (Na₂SO₄) and concentrated. Column chromatography (SiO₂; CH₂Cl₂/EtOAc 49:1→24:1→19:1→9:1→4:1) gives the title compound: MS: [M+1]⁺=437; HPLC: t_{Ret}=1.37; TLC(CH₂Cl₂/EtOAc 9:1): R_{f}=0.18.

### Step 17.2: (6-Amino-8-naphthalen-1-yl-quinolin-2-yl)-phosphonic acid diethyl ester

(8-Naphthalen-1-yl-6-nitro-quinolin-2-yl)-phosphonic acid diethyl ester (5.9 g, 13.5 mMol) in 300 ml THF is hydrogenated in presence of 4 g Raney-Nickel (in EtOH; B113 W Degussa). The catalyst is filtered off through Hyflo and washed with THF. Concentration of the filtrate and crystallization from DIPE gives the title compound: MS: [M+1]⁺=407; HPLC: t_{Ret}=1.13; TLC(CH₂Cl₂/EtOAc 1:1): R_{f}=0.34.

### Step 17.3: (6-Iodo-8-naphthalen-1-yl-quinolin-2-yl)-phosphonic acid diethyl ester

To (6-amino-8-naphthalen-1-yl-quinolin-2-yl)-phosphonic acid diethyl ester (4.8 g, 11.8 mMol) in 73 ml conc. HCl, pieces of ice are added and the mixture is cooled to −15° C. Then a solution of NaNO₂ (1.63 g, 23.6 mMol) in 96 ml H₂O is added during 20 min. After stirring for 30 min, the reddish solution is added to a solution of KI (106 g, 638 mMol) in 544 ml H₂O during 7 min. The brown suspension is warmed up to rt and stirred for 5 h and then diluted with EtOAc and water. The aq. phase is separated off and extracted twice with EtOAc. The organic layers are washed with 2 N NaOH, 10% Na₂S₂O₃ solution and brine, dried (Na₂SO₄) concentrated. Column chromatography (SiO₂; CH₂Cl₂/EtOAc 9:1→7:3) and crystallization from hexane gives the title compound: m.p.: 127-128° C.; Anal.: C, H, N, I, P; MS: [M+1]⁺=518.

### Step 17.4: (8-Naphthalen-1-yl-6-thiophen-2-yl-quinolin-2-yl)-phosphonic acid diethyl ester

A solution of (6-iodo-8-naphthalen-1-yl-quinolin-2-yl)-phosphonic acid diethyl ester (250 mg, 0.483 mMol), 2-thiophene boronic acid (74.2 mg, 0.58 mMol) and 1.1 ml 1 M aq. K₂CO₃ in 2.5 ml DMF is degassed by repeated evacuation by HV and flushing with N₂. Then (Ph₃P)₂PdCl₂ (20.6 mg, 0.029 mMol) is added and it is heated up to 85° C. for 1¼ h. The cold mixture is poured into brine and extracted three times with EtOAc. The organic layers are washed with water and brine, dried (Na₂SO₄) and concentrated. Reversed phase chromatography gives the title compound: MS: [M+1]⁺=474; HPLC: t_{Ret}=1.54; TLC(CH₂Cl₂/EtOAc 4:1): R_{f}=0.50.

### EXAMPLE 18

### (8-Naphthalen-1-yl-6-nitro-quinolin-2-yl)-phosphonic

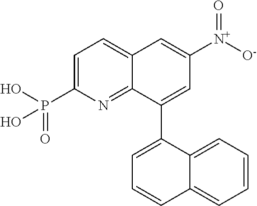

The title compound is obtained analogously to Ex. 4 starting from (8-naphthalen-1-yl-6-nitro-quinolin-2-yl)-phosphonic acid diethyl ester (282 mg, 0.646 mMol): Anal. (+0.03 HBr+0.5; H₂O): C, H, N, Br, P; MS: [M+1]⁺=381; HPLC: t_{Ret}=0.98.

### EXAMPLE 19

### (8-Naphthalen-1-yl-6-nitro-quinolin-2-yl)-phosphonic acid monoethyl ester

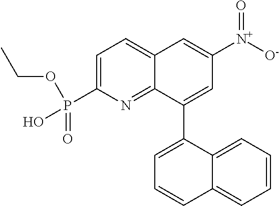

To a suspension of 2-chloro-8-naphthalen-1-yl-6-nitro-quinoline (167.4 mg, 0.50 mMol; Step 5.3) in 1.5 ml degassed toluene, Et₃N (77 μl, 0.55 mMol), diethylphosphite (71 μl, 0.55 mMol), ⁿBu₄NI (203 mg, 0.55 mMol) and (Ph₃P)₄Pd (58 mg, 0.05 mMol) are added. This mixture is stirred for 19 h at 100° C. in a sealed vessel, giving a mixture of (8-naphthalen-1-yl-6-nitro-quinolin-2-yl)-phosphonic acid diethyl ester and (8-naphthalen-1-yl-6-nitro-quinolin-2-yl)-phosphonic acid monoethyl ester. Separation by reversed phase chromatography gives the title compound: MS: [M+1]⁺=409; HPLC: t_{Ret}=1.12; TLC(EtOAc/EtOH/HOAc 200:200:1): R_{f}=0.29.

### EXAMPLE 20

### (6-Amino-8-naphthalen-1-yl-quinolin-2-yl)-phosphonic acid

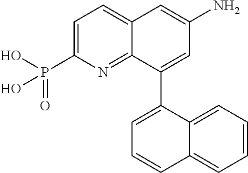

A solution of (6-amino-8-naphthalen-1-yl-quinolin-2-yl)-phosphonic acid diethyl ester (134 mg, 0.33 mMol) in 10 ml CH₂Cl₂ is cooled in an icebath. Then bromo-trimethyl-silane (427 μl, 3.3 mMol) is added and the orange solution is stirred for 5 h at it in a sealed vessel. The mixture is concentrated in vacuo and the residue triturated from hot EtOAc, yielding the title compound as the hydrobromide salt: Anal. (+1.08HBr+2.3; H₂O+0.8 EtOAc): C, H, N, Br, P; MS: [M+1]⁺=351; HPLC: t_{Ret}=0.73.

### EXAMPLE 21

### (6-Iodo-8-naphthalen-1-yl-quinolin-2-yl)-phosphonic acid

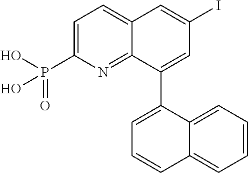

(6-Iodo-8-naphthalen-1-yl-quinolin-2-yl)-phosphonic acid diethyl ester (170 mg, 0.329 mMol) is deprotected as described in Ex. 20, yielding the title compound as the hydrobromide salt: Anal. (+0.9HBr+1.5; H₂O+0.75 EtOAc): C, H, N, Br, I, P; MS: [M+1]⁺=462; HPLC: t_{Ret}=1.04.

### EXAMPLE 22

### 2-(8-Naphthalen-1-yl-2-phosphono-quinolin-6-yl)-pyrrole-1-carboxylic acid tert-butyl ester

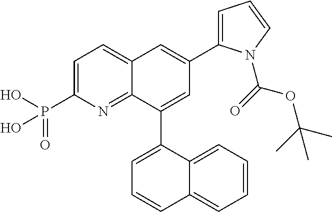

A solution of 2-[2-(diethoxy-phosphoryl)-8-naphthalen-1-yl-quinolin-6-yl]-pyrrole-1-carboxylic acid tert-butyl ester (95 mg, 0.171 mMol) in 5 ml CH₂Cl₂ is cooled in an icebath. Then bromo-trimethyl-silane (221 μl, 1.71 mMol) is added and the orange solution is stirred for 4 h at rt in a sealed vessel. The mixture is concentrated in vacuo and the residue triturated from EtOAc and filtered: The precipitate consists of a mixture of the title compound and [8-naphthalen-1-yl-6-(1H-pyrrol-2-yl)-quinolin-2-yl]-phosphonic acid. Concentration and crystallization of the mother liquor from ether gives the pure title compound: MS: [M+1]⁺=501; HPLC: t_{Ret}=1.18; IR [cm⁻¹]: 1743s, 1323s, 1142s.

The starting material is prepared as follows:

### Step 22.1: 2-[2-(Diethoxy-phosphoryl)-8-naphthalen-1-yl-quinolin-6-yl]-pyrrole-1-carboxylic acid tert-butyl ester A and [8-naphthalen-1-yl-6-(1H-pyrrol-2-yl)-quinolin-2-yl]-phosphonic acid diethyl ester B

A suspension of (6-iodo-8-naphthalen-1-yl-quinolin-2-yl)-phosphonic acid diethyl ester (250 mg, 0.483 mMol), 1-N—BOC-pyrrole-2-boronic acid (122 mg, 0.58 mMol) and 1.1 ml 1 M aq. K₂CO₃ in 2.5 ml DMF is degassed by repeated evacuation by HV and flushing with N₂. Then (Ph₃P)₂PdCl₂ (20.6 mg, 0.029 mMol) is added and it is heated up to 110° C. for 50 min. The cold mixture is poured into brine and extracted three times with EtOAc. The organic layers are washed with water and brine, dried (Na₂SO₄) and concentrated. Chromatography (Combi Flash; CH₂Cl₂/EtOAc 9:1→4:1) gives A, followed by B: A: MS: [M+1]⁺=557; HPLC: t_{Ret}=1.60; TLC(CH₂Cl₂/EtOAc 4:1): R_{f}=0.45. B: MS: [M+1]⁺=457; HPLC: t_{Ret}=1.38; TLC(CH₂Cl₂/EtOAc 4:1): R_{f}=0.19.

### EXAMPLE 23

### [8-Naphthalen-1-yl-6-(1H-pyrrol-2-yl)-quinolin-2-yl]-phosphonic acid

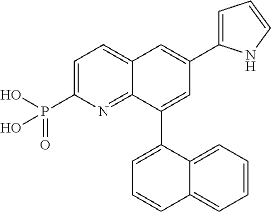

The mixture of 2-(8-naphthalen-1-yl-2-phosphono-quinolin-6-yl)-pyrrole-1-carboxylic acid tert-butyl ester and [8-naphthalen-1-yl-6-(1H-pyrrol-2-yl)-quinolin-2-yl]-phosphonic acid (precipitate Ex. 22) is stirred in HCl in dioxane (2 N; 6 ml). Concentration and reversed phase chromatography gives the title compound: MS: [M+1]⁺=401; HPLC: t_{Ret}=0.92.

### EXAMPLE 24

### [6-(1H-Indol-2-yl)-8-naphthalen-1-yl-quinolin-2-yl]-phosphonic acid

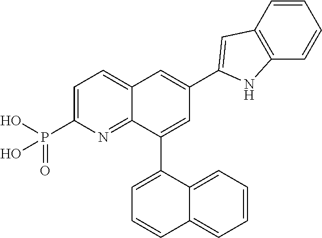

A solution of 2-[2-(diethoxy-phosphoryl)-8-naphthalen-1-yl-quinolin-6-yl]-indole-1-carboxylic acid tert-butyl ester (150 mg, 0.247 mMol) in 7 ml CH₂Cl₂ is cooled in an icebath. Then bromo-trimethyl-silane (319 μl, 2.47 mMol) is added and the orange solution is stirred for 2 h at rt in a sealed vessel. Precipitation with hexane, collection of the solid and reversed phase chromatography gives the title compound: MS: [M+1]⁺=451; HPLC: t_{Ret}=1.08.

The starting material is prepared as follows:

### Step 24.1: 2-[2-(Diethoxy-phosphoryl)-8-naphthalen-1-yl-quinolin-6-yl]-indole-1-carboxylic acid tert-butyl ester A and [6-(1H-indol-2-yl)-8-naphthalen-1-yl-quinolin-2-yl]-phosphonic acid diethyl ester B

(6-Iodo-8-naphthalen-1-yl-quinolin-2-yl)-phosphonic acid diethyl ester (250 mg, 0.483 mMol) and 1-N—BOC-indole-2-boronic acid (151 mg, 0.58 mMol) are converted to A and B as described in Step 22.1: A: MS: [M+1]⁺=607; HPLC: t_{Ret}=1.79; TLC(CH₂Cl₂/EtOAc 4:1): R_{f}=0.42. B: MS: [M+1]⁺=507; HPLC: t_{Ret}=1.62; TLC(CH₂Cl₂/EtOAc 4:1): R_{f}=0.19.

### EXAMPLE 25

### [6-(6-Methoxy-pyridin-3-yl)-8-naphthalen-1-yl-quinolin-2-yl]-phosphonic acid A and [6-(6-hydroxy-pyridin-3-yl)-8-naphthalen-1-yl-quinolin-2-yl]-phosphonic acid B

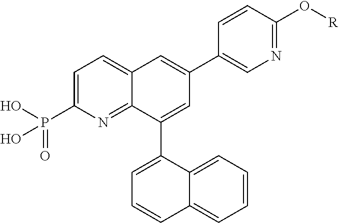

A solution of [6-(6-methoxy-pyridin-3-yl)-8-naphthalen-1-yl-quinolin-2-yl]-phosphonic acid diethyl ester (135 mg, 0.271 mMol) in 7 ml CH₂Cl₂ is cooled in an icebath. Then bromo-trimethyl-silane (350 μl, 2.71 mMol) is added and the orange solution is stirred for 3 h at rt in a sealed vessel. Concentration in vacuo and reversed phase chromatography gives A and B as the TFA-salts: A: MS: [M+1]⁺=443; HPLC: t_{Ret}=1.10. B: MS: [M+1]⁺=429; HPLC: t_{Ret}=0.86.

The starting material is prepared as follows:

### Step 25.1: [6-(6-Methoxy-pyridin-3-yl)-8-naphthalen-1-yl-quinolin-2-yl]-phosphonic acid diethyl ester

(6-Iodo-8-naphthalen-1-yl-quinolin-2-yl)-phosphonic acid diethyl ester (250 mg, 0.483 mMol) and 2-methoxypyridin-5-yl-boronic acid (88.7 mg, 0.58 mMol) are converted to the title compound as described in Step 22.1: MS: [M+1]⁺=499; HPLC: t_{Ret}=1.47; TLC(CH₂Cl₂/EtOAc 1:1): R_{f}=0.22.

### EXAMPLE 26

### [8-Naphthalen-1-yl-6-(1H-pyrrol-3-yl)-quinolin-2-yl]-phosphonic acid

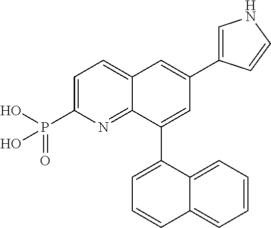

The title compound is obtained analogously to Ex. 17 starting from [8-naphthalen-1-yl-6-(1H-pyrrol-3-yl)-quinolin-2-yl]-phosphonic acid diethyl ester (94 mg, 0.206 mMol): MS: [M+1]⁺=401; HPLC: t_{Ret}=0.83.

The starting material is prepared as follows:

### Step 26.1: [8-Naphthalen-1-yl-6-[1-(triisopropylsilyl)-1H-pyrrol-3-yl]-quinolin-2-yl]-phosphonic acid diethyl ester A and [8-naphthalen-1-yl-6-(1H-pyrrol-3-yl)-quinolin-2-yl]-phosphonic acid diethyl ester B

A suspension of (6-iodo-8-naphthalen-1-yl-quinolin-2-yl)-phosphonic acid diethyl ester (250 mg, 0.483 mMol), 1-(triisopropylsilyl)-1H-pyrrole-3-boronic acid (155 mg, 0.58 mMol) and 1.1 ml 1 M aq. K₂CO₃ in 2.5 ml DMF is degassed by repeated evacuation by HV and flushing with N₂. Then (Ph₃P)₂PdCl₂ (20.6 mg, 0.029 mMol) is added and it is heated up to 85° C. for 30 min. The cold mixture is poured into brine and extracted three times with EtOAc. The organic layers are washed with water and brine, dried (Na₂SO₄) and concentrated. Chromatography (Combi Flash; CH₂Cl₂/EtOAc 49:1→9:1) gives A, followed by B: A: MS: [M+1]⁺=613; HPLC: t_{Ret}=1.89; TLC(CH₂Cl₂/EtOAc 4:1): R_{f}=0.41. B: MS: [M+1]⁺=457; HPLC: t_{Ret}=1.31; TLC(CH₂Cl₂/EtOAc 4:1): R_{f}=0.22.

### EXAMPLE 27

the following derivatives are obtained analogously to Step 22.1; 26.1; Ex. 17:

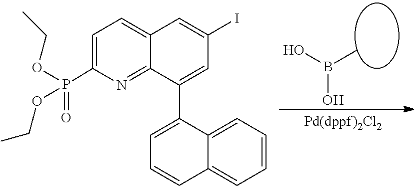

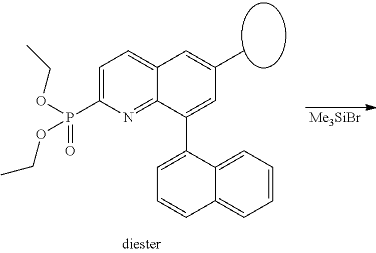

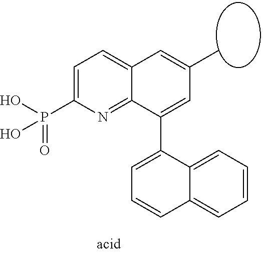

| Ex. 27. | 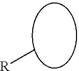 | TLC R_{f} | MS [M + 1]⁺ | HPLC t_{Ret} | salt form |
|---|---|---|---|---|---|
| a.i diester a.ii acid | 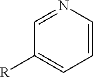 | 0.26¹⁾ | 469 413 | 1.01 0.63 | 1 HBr |
| b.i diester b.ii acid | 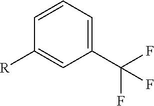 | 0.19²⁾ | 536 480 | 1.62 1.15 | no salt |
| c.i dieseter c.ii acid | 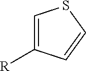 | 0.39³⁾ | 474 418 | 1.54 1.00 | no salt |
| d.i diester d.ii acid | 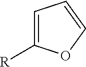 | 0.28³⁾ | 458 402 | 1.49 0.96 | no salt |
| e.i diester e.ii acid | 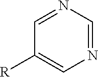 | 0.13⁴⁾ | 470 414 | 1.22 0.76 | 1 HBr |
| f.i diester f.ii acid | 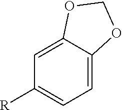 | 0.25³⁾ | 512 456 | 1.51 1.00 | no salt |
| g.i dieseter g.ii acid | 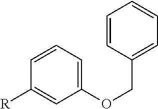 | 0.36³⁾ | 574 518 | 1.68 1.23 | no salt |

¹⁾EtOAc/EtOH 9:1;
²⁾CH₂Cl₂/EtOAc 9:1;
³⁾CH₂Cl₂/EtOAc 4:1;
⁴⁾CH₂Cl₂/EtOAc/EtOH 10:9:1

### EXAMPLE 28

### [6-(3-Hydroxy-phenyl)-8-naphthalen-1-yl-quinolin-2-yl]-phosphonic acid

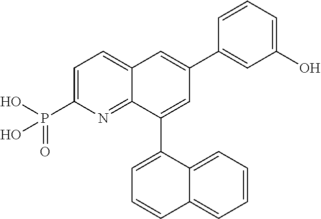

The title compound is obtained analogously to Ex. 17 after deprotection of [6-(3-hydroxy-phenyl)-8-naphthalen-1-yl-quinolin-2-yl]-phosphonic acid diethyl ester (49 mg, 0.101 mMol) by bromo-trimethyl-silane (65 μl, 0.50 mMol) during 20 h at rt: Anal. (+0.1 HBr+3.4; H₂O): C, H, N, Br; MS: [M+1]⁺=428; HPLC: t_{Ret}=0.87.

The starting material is prepared as follows:

### Step 28.1: [6-(3-Hydroxy-phenyl)-8-naphthalen-1-yl-quinolin-2-yl]-phosphonic acid diethyl ester

A solution of [6-(3-benzyloxy-phenyl)-8-naphthalen-1-yl-quinolin-2-yl]-phosphonic acid diethyl ester (260 mg, 0.453 mMol; Ex. 27g) in 10 ml THF is hydrogenated in presence of 80 mg Pd/C (10%, Engelhard 4505). The catalyst is filtered off and the filtrate concentrated. The residue is re-dissolved in 7 ml benzene and after addition of 2,3-dichloro-5,6-dicyano-p-benzochinone (209 mg, 0.92 mMol) stirred for 30 min under reflux. Concentration, chromatography (Combi Flash; CH₂Cl₂/EtOAc 49:1→1:1) and treatment with char coal gives the title compound: MS: [M+1]⁺=484; HPLC: t_{Ret}=1.36; TLC(CH₂Cl₂/EtOAc 1:1): R_{f}=0.31.

### EXAMPLE 29

### 6-Ethoxycarbonylamino-8-naphthalen-1-yl-quinoline-2-carboxylic acid

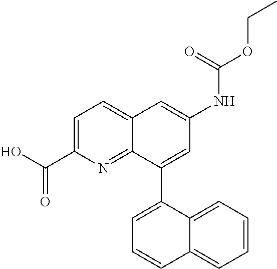

To a solution of 6-ethoxycarbonylamino-8-naphthalen-1-yl-quinoline-2-carboxylic acid ethyl ester (70 mg, 0.169 mMol) in 1.1 ml dioxane, 200 μl of a 1 M aq. LiOH solution are added. It is stirred for 3 h at rt and then concentrated in vacuo. Chromatography (Combi Flash; CH₂Cl₂→CH₂Cl₂/(EtOH+2% HOAc) 9:1) gives the title compound: m.p.: 205-208° C.; MS: [M+1]⁺=387; HPLC: t_{Ret}=1.19; ¹H-NMR (DMSO-d₆): δ ppm 10.22 (s, HN), 8.48 (d, 1H), 8.34 (s, 1H), 8.02 (m, 3H), 7.77 (s, 1H), 7.62 (t, 1H), 7.48 (m, 2H), 7.33 (t, 1H), 7.27 (d, 1H), 4.19 (q, H₂C), 1.28 (t, H₃C).

The starting material is prepared as follows:

### Step 29.1: 6-Ethoxycarbonylamino-8-naphthalen-1-yl-quinoline-2-carboxylic acid ethyl ester A; 6-amino-5-ethoxy-8-naphthalen-1-yl-quinoline-2-carboxylic acid ethyl ester B and 6-amino-8-naphthalen-1-yl-quinoline-2-carboxylic acid ethyl ester C

To 2-chloro-8-naphthalen-1-yl-6-nitro-quinoline (9.4 g, 28.1 mMol; Step 5.3) in 120 ml EtOH and Et₃N (7.9 ml, 56 mMol), (Ph₃P)₂PdCl₂ (1.97 g, 2.8 mMol) is added. This mixture is heated up to 110° C. in an autoclave under a CO-atmosphere of 120 bar for 24 h. After 8 h and 16 h, additional portions of 1.97 g and 3.0 g of (Ph₃P)₂PdCl₂ are added. The reaction mixture is diluted with EtOAc and water, the aq. layer separated off and extracted twice with EtOAc. The organic layers are washed 2× with water and brine, dried (Na₂SO₄) and concentrated. Column chromatography (SiO₂; hexane/EtOAc 2:1) successively gives a mixture of A and B (AB), followed by a mixture of B and C (BC) and finally C. Reversed phase chromatography of AB gives B and A: A: MS: [M+1]⁺=415; HPLC: t_{Ret}=1.39; ¹H-NMR (DMSO-d₆): δ ppm 10.25 (s, HN), 8.52 (d, 1H), 8.35 (s, 1H), 8.02 (m, 3H), 7.81 (s, 1 H), 7.62 (t, 1H), 7.49 (m, 2H), 7.33 (t, 1H), 7.31 (d, 1H), 4.19 (q, H₂C), 4.16 (m, H₂C), 1.27 (t, H₃C), 1.12 (t, H₃C). B: MS: [M+1]⁺=387; HPLC: t_{Ret}=1.35; ¹H-NMR (DMSO-d₆): δ ppm 8.35 (d, 1H), 7.98 (m, 2H), 7.93 (d, 1H), 7.58 (t, 1H), 7.48 (t, 1H), 7.45 (d, 1H), 7.38 (s, 1 H), 7.33 (m, 2H), 5.87 (s, H₂N), 4.10 (m, H₂C), 4.02 (q, H₂C), 1.49 (t, H₃C), 1.10 (t, H₃C). C: MS: [M+1]⁺=343; HPLC: t_{Ret}=1.19; ¹H-NMR (DMSO-d₆): δ ppm 8.13 (d, 1H), 7.98 (m, 2 H), 7.84 (d, 1H), 7.59 (t, 1H), 7.48 (t, 1H), 7.45 (d, 1H), 7.33 (m, 2H), 7.22 (s, 1H), 6.93 (s, 1H), 6.11 (s, H₂N), 4.10 (m, H₂C), 1.11 (t, H₃C).

### EXAMPLE 30

### 6-Amino-5-ethoxy-8-naphthalen-1-yl-quinoline-2-carboxylic acid

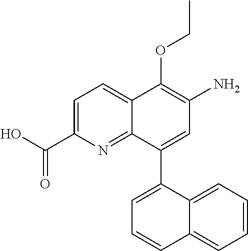

Prepared as described in Ex. 29 from 6-amino-5-ethoxy-8-naphthalen-1-yl-quinoline-2-carboxylic acid ethyl ester (100 mg, 0.259 mMol): m.p.: 182-184° C.; MS: [M+1]⁺=359; HPLC: t_{Ret}=1.12; ¹H-NMR (Li-salt; DMSO-d₆): δ ppm 8.18 (d, 1H), 7.95 (d, 1 H), 7.92 (d, 1H), 7.83 (d, 1H), 7.54 (t, 1H), 7.45 (t, 1H), 7.39 (d, 1H), 7.29 (t, 1H), 7.25 (d, 1H), 7.12 (s, 1H), 5.34 (s, H₂N), 4.01 (q, H₂C), 1.47 (t, H₃C).

### EXAMPLE 31

### 6-Amino-8-naphthalen-1-yl-quinoline-2-carboxylic acid

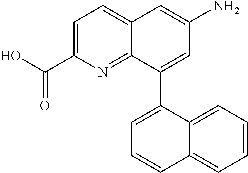

Prepared as describe in Ex. 29 from 6-amino-8-naphthalen-yl-quinoline-2-carboxylic acid ethyl ester (100 mg, 0.292 mMol) and isolated as its TFA-salt via reversed phase chromatography: Anal. (+0.8 TFA+0.5; H₂O+0.1 dioxane): C, H, N, F; MS: [M+1]⁺=315; HPLC: t_{Ret}=0.93; ¹H-NMR (Li-salt; DMSO-d₆): δ ppm 8.13 (d, 1H), 7.98 (m, 2H), 7.86 (d, 1H), 7.59 (t, 1H), 7.48 (m, 1H), 7.45 (d, 1H), 7.33 (m, 2H), 7.21 (s, 1H), 6.95 (s, 1H).

### EXAMPLE 32

### 6-Iodo-8-naphthalen-1-yl-quinoline-2-carboxylic acid lithium salt

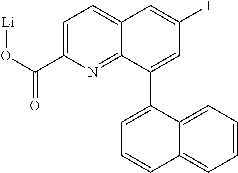

Saponification of 6-iodo-8-naphthalen-1-yl-quinoline-2-carboxylic acid ethyl ester (90 mg, 0.199 mMol) as described in Ex. 29 leads to precipitation of the title compound, which can be isolated by filtration and washing with dioxane/DIPE 1:1: Anal. (+1.4; H₂O): C, H, N, Li; MS: [M+1]⁺=426; HPLC: t_{Ret}=1.43.

The starting material is prepared as follows:

### Step 32.1: 6-Iodo-8-naphthalen-1-yl-quinoline-2-carboxylic acid ethyl ester

6-Amino-8-naphthalen-1-yl-quinoline-2-carboxylic acid ethyl ester (364 mg, 1.06 mMol) and pieces of ice in 6.6 ml conc. HCl are cooled to −15° C. Then a solution of NaNO₂ (146 mg, 2.1 mMol) in 8.7 ml H₂O is added dropwise and the mixture is stirred for 20 min. The suspension is added portion wise to an ice cooled solution of KI (9.6 g, 58 mMol) in 40 ml H₂O. After 15 min at 0° C., the mixture is diluted with EtOAc and water. The aq. phase is separated off and extracted twice with EtOAc. The organic layers are washed with water, diluted Na₂S₂O₃ solution and brine, dried (Na₂SO₄) and concentrated. Chromatography (Combi Flash; toluene toluene/CH₂Cl₂ 7:3) gives the title compound: MS: [M+1]⁺=454; HPLC: t_{Ret}=1.59; TLC(toluene): R_{f}=0.09.

### EXAMPLE 33

### 5-Ethoxy-6-iodo-8-naphthalen-1-yl-quinoline-2-carboxylic acid lithium salt

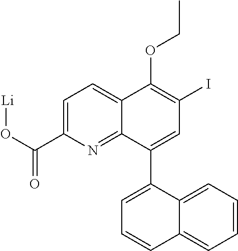

Saponification of 5-ethoxy-6-iodo-8-naphthalen-1-yl-quinoline-2-carboxylic acid ethyl ester (100 mg, 0.20 mMol) as described in Ex. 29 leads to precipitation of the title compound, which can be isolated by filtration and washing with dioxane/DIPE 1:1: Anal. (+1.7; H₂O): C, H, N, Li; MS: [M+1]⁺=470; HPLC: t_{Ret}=1.50.

The starting material is prepared as follows:

### Step 33.1: 5-Ethoxy-6-iodo-8-naphthalen-1-yl-quinoline-2-carboxylic acid ethyl ester

Prepared from a mixture of 6-amino-5-ethoxy-8-naphthalen-1-yl-quinoline-2-carboxylic acid ethyl ester and 6-amino-8-naphthalen-1-yl-quinoline-2-carboxylic acid ethyl ester (BC; Step 29.1) as described in Step 32.1 and column chromatography (SiO₂; toluene→toluene/CH₂Cl₂ 19:1→23:2→4:1) gives the title compound: MS: [M+1]⁺=498; HPLC: t_{Ret}=1.65.

### EXAMPLE 34

### 8-Naphthalen-1-yl-6-thiophen-3-yl-quinoline-2-carboxylic acid

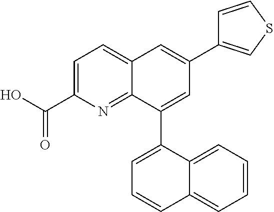

To a solution of 8-naphthalen-1-yl-6-thiophen-3-yl-quinoline-2-carboxylic acid ethyl ester (86 mg, 0.21 mMol) in 2 ml dioxane, 220 μl of a 1 M aq. LiOH solution are added. It is stirred for 18 h at rt and after addition of 0.7 g SiO₂ concentrated in vacuo. The resulting powder is put on a Combi Flash chromatography column and the title compound eluted [CH₂Cl₂→CH₂Cl₂/(EtOH+2% HOAc) 9:1]: m.p.: 164-166° C.; MS: [M+1]⁺=382; HPLC: t_{Ret}=1.43; ¹H-NMR (DMSO-d₆): δ ppm 13.05 (s, HOOC), 8.61 (d, 1H), 8.54 (s, 1H), 8.22 (m, 2H), 8.08 (d, 1H), 8.03 (d, 1H), 8.01 (d, 1H), 7.83 (d, 1H), 7.73 (dd, 1H), 7.64 (t, 1H), 7.55 (d, 1H), 7.50 (m, 1H), 7.32 (m, 2H).

The starting material is prepared as follows:

### Step 34.1: 8-Naphthalen-1-yl-6-thiophen-3-yl-quinoline-2-carboxylic acid ethyl ester

A solution of 6-iodo-8-naphthalen-1-yl-quinoline-2-carboxylic acid ethyl ester (129 mg, 0.28 mMol) in 1.3 ml DMF is degassed by repeated evacuation by HV and flushing with N₂. Then thiophen-3-boronic acid (71.7 mg, 0.56 mMol), anhydrous Na₂CO₃ (59.4 mg, 0.56 mMol) and (Ph₃P)₂PdCl₂ (12 mg, 0.017 mMol) are added. This mixture is then stirred in a pre-heated oil bath of 100° C. for 100 min. The cold mixture is poured into water and EtOAc, the aq. layer separated off and extracted twice with EtOAc. The organic layers are washed with brine, dried (Na₂SO₄) and concentrated. Chromatography (Combi Flash; toluene→toluene/CH₂Cl₂ 3:2) yields the title compound: MS: [M+1]⁺=410; HPLC: t_{Ret}=1.57; TLC(CH₂Cl₂): R_{f}=0.41.

### EXAMPLE 35

### 8-Naphthalen-1-yl-6-thiophen-2-yl-quinoline-2-carboxylic acid lithium salt

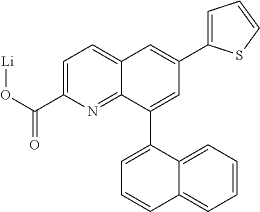

Saponification of 8-naphthalen-1-yl-6-thiophen-2-yl-quinoline-2-carboxylic acid ethyl ester (60 mg, 0.147 mMol) in 1.5 ml dioxane as described in Ex. 29 leads to precipitation of the title compound, which can be isolated by filtration and washing with DIPE: Anal. (+1.7; H₂O): C, H, N, S, Li; MS: [M+1]⁺=382; HPLC: t_{Ret}=1.45.

The starting material is prepared as follows:

### Step 35.1: 8-Naphthalen-1-yl-6-thiophen-2-yl-quinoline-2-carboxylic acid ethyl ester

Prepared as described in Step 34.1 from 6-iodo-8-naphthalen-1-yl-quinoline-2-carboxylic acid ethyl ester (125 mg, 0.276 mMol) and thiophen-2-boronic acid (70.6 mg, 0.552 mMol) in 2 ml DMF: MS: [M+1]⁺=410; HPLC: t_{Ret}=1.59; TLC(CH₂Cl₂/hexane 9:1): R_{f}=0.40.

### EXAMPLE 36

### 8-Naphthalen-1-yl-6-pyrrol-2-yl-quinoline-2-carboxylic acid

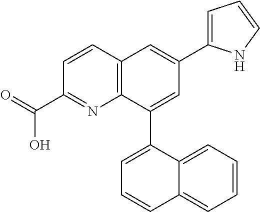

A mixture of 6-(1-tert-butoxycarbonyl-1H-pyrrol-2-yl)-8-naphthalen-1-yl-quinoline-2-carboxylic acid ethyl ester (42 mg, 0.072 mMol), 2 ml THF and 2 ml 4 N aq. HCl is stirred for 12 h at 50° C. Then it is diluted with water and EtOAc, the aq. layer separated off and extracted twice with EtOAc. The organic layers are washed with brine, dried (Na₂SO₄) and concentrated. Reversed phase chromatography gives the title compound: MS: [M+1]⁺=365; HPLC: t_{Ret}=1.28; TLC(CH₂Cl₂/MeOH 9:1): R_{f}=0.23.

The starting material is prepared as follows:

### Step 36.1: 6-(1-tert-Butoxycarbonyl-1H-pyrrole-2-yl)-8-naphthalen-1-yl-quinoline-2-carboxylic acid ethyl ester

Prepared as described in Step 34.1 from 6-iodo-8-naphthalen-1-yl-quinoline-2-carboxylic acid ethyl ester (128 mg, 0.282 mMol) and 1-N-Boc-pyrrole-2-boronic acid (119 mg, 0.564 mMol) in 2 ml DMF: MS: [M+1]⁺=493; HPLC: t_{Ret}=1.66; TLC(CH₂Cl₂/EtOAc/hexane 9:1:10): R_{f}=0.40.

### EXAMPLE 37

### 8-(5-Hydroxymethyl-naphthalen-1-yl)-quinoline-2-carboxylic acid

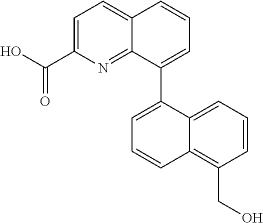

Prepared as described in Ex. 29 from 8-(5-hydroxymethyl-naphthalen-1-yl)-quinoline-2-carboxylic acid methyl ester (32 mg, 0.093 mMol): MS: [M+1]⁺=330; HPLC: t_{Ret}=1.00; TLC(CH₂Cl₂/EtOH/HOAc 450:50:1): R_{f}=0.27.

The starting material is prepared as follows:

### Step 37.1: 8-Trifluoromethanesulfonyloxy-quinoline-2-carboxylic acid methyl ester

8-Hydroxy-quinoline-2-carboxylic acid methyl ester (2.03 g, 10.0 mMol) is dissolved in 100 ml CH₂Cl₂ and cooled to −78° C. Then Et₃N (4.3 ml, 31 mMol) is added, followed by a solution of (F₃CSO₂)₂O (2.4 ml, 14 mMol) in 10 ml CH₂Cl₂. After 3 h at −78° C., the mixture is poured into a mixture of EtOAc and water/sat. NaHCO₃ 10:1. The aq. phase is separated off and extracted twice with EtOAc. The organic layers are washed with water and brine, dried (Na₂SO₄), concentrated and crystallized from DIPE/hexane gives the title compound: m.p.: 77-78° C.; MS: [M+1]⁺=336.

### Step 37.2: 8-(5-Hydroxymethyl-naphthalen-1-yl)-quinoline-2-carboxylic acid methyl ester

8-Trifluoromethanesulfonyloxy-quinoline-2-carboxylic acid methyl ester (1.0 g, 2.98 mMol) is dissolved in 14 ml dry DMF. Then bis-(pinacolato)-diboron (909 mg, 3.58 mMol), potassium acetate (878 mg, 8.95 mMol) and 4 g molecular sieves 4 Å are added. After degassing the mixture by repeated evacuation and flushing with N₂, [1,1′-bis(diphenylphosphino)ferrocene]palladium(II) chloride, complex with CH₂Cl₂, (36.5 mg, 0.045 mMol) is added. The reaction mixture is heated for 4½ h at 80° C. without stirring, filtered and the filtrate diluted with brine and EtOAc. The aq. phase is separated off and extracted twice with EtOAc. The organic layers are washed with water and brine, dried (Na₂SO₄) and concentrated (1.1 g boronate).

To 229 mg of this boronate in 6 ml degassed toluene, 1-bromo-5-hydroxymethyl-naphthaline (196 mg, 0.827 mMol), K₂CO₃ (206 mg, 1.49 mMol) and (Ph₃P)₄Pd (46 mg, 0.04 mMol) is added. The reaction mixture is stirred for 18 h at 90° C., diluted with water and EtOAc, the aq. phase separated off and extracted twice with EtOAc. The organic layers are washed with water and brine, dried (Na₂SO₄) and concentrated. Chromatography (Combi Flash; CH₂Cl₂→CH₂Cl₂/acetone 19:1) gives the title compound: MS: [M+1]⁺=344; TLC(CH₂Cl₂/acetone 19:1): Rf=0.17.

### EXAMPLE 38

### 8-(5-Methyl-naphthalen-1-yl)-quinoline-2-carboxylic acid

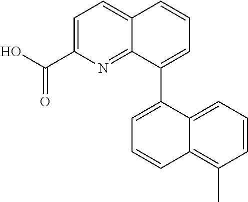

Prepared as described in Ex. 29 from 8-(5-methyl-naphthalen-1-yl)-quinoline-2-carboxylic acid methyl ester (9 mg, 0.027 mMol): MS: [M+1]⁺=314; HPLC: t_{Ret}=1.31; TLC(CH₂Cl₂/EtOH/HOAc 450:50:1): R_{f}=0.51.

The starting material is prepared as follows:

### Step 38.1: 8-(5-Acetoxymethyl-naphthalen-1-yl)-quinoline-2-carboxylic acid methyl ester

A solution of 8-(5-hydroxymethyl-naphthalen-1-yl)-quinoline-2-carboxylic acid methyl ester (99 mg, 0.288 mMol), 0.1 ml acetic acid anhydride and a trace of DMAP in 2 ml CH₂Cl₂ and 0.2 ml pyridine is stirred for 16 h at rt and then diluted with EtOAc and water. The aq. phase is separated off and extracted twice with EtOAc. The organic layers are washed with water and brine, dried (Na₂SO₄), concentrated to the crude title compound: MS: [M+1]⁺=386.

### Step 38.2: 8-(5-Methyl-naphthalen-1-yl)-quinoline-2-carboxylic acid methyl ester

The crude 8-(5-acetoxymethyl-naphthalen-1-yl)-quinoline-2-carboxylic acid methyl ester from Step 38.1 is dissolved in 10 ml MeOH and hydrogenated in presence of 60 mg Pd/C (5%, E101N/D Degussa). The catalyst is then filtered off, washed with MeOH and the filtrate is concentrated. This residue is re-dissolved in 5 ml benzene and after addition of 2,3-dichloro-5,6-dicyano-p-benzochinone (132 mg, 0.58 mMol) stirred for 30 min under reflux. Concentration after addition of SiO₂ and chromatography (Combi Flash; CH₂Cl₂/hexane 1:9→CH₂Cl₂) gives the title compound: MS: [M+1]⁺=328; HPLC: t_{Ret}=1.43; TLC(CH₂Cl₂): R_{f}=0.42.

### EXAMPLE 39

### 8-(5-Amino-naphthalen-1-yl)-quinoline-2-carboxylic acid

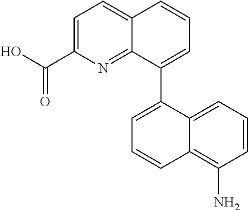

Prepared as described in Ex. 29 from 8-(5-amino-naphthalen-1-yl)-quinoline-2-carboxylic acid methyl ester (50 mg, 0.15 mMol) and isolated by reversed phase chromatography as trifluoracetate salt: Anal. (+1.15 TFA+0.8; H₂O): C, H, N, F; MS: [M+1]⁺=315; HPLC: t_{Ret}=0.81.

The starting material is prepared as follows:

### Step 39.1: 8-(5-Amino-naphthalen-1-yl)-quinoline-2-carboxylic acid methyl ester

Prepared as described in Step 37.2 from 0.15 g boronate, 1-amino-5-bromo-naphthaline (117 mg, 0.526 mMol), K₂CO₃ (110 mg, 0.79 mMol) and (Ph₃P)₄Pd (24.3 mg, 0.021 mMol) in 2.5 ml toluene: MS: [M+1]⁺=329; HPLC: t_{Ret}=0.94; TLC(hexane/EtOAc 1:1): Rf=0.24.

### EXAMPLE 40

### [(E)-2-(8-Naphthalen-1-yl-quinolin-2-yl)-vinyl]-phosphonic acid

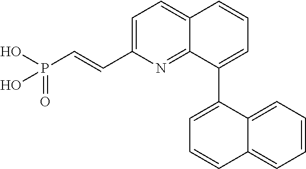

To a solution of [(E)-2-(8-naphthalen-1-yl-quinolin-2-yl)-vinyl]-phosphonic acid diethyl ester (100 mg, 0.24 mMol) in 10 ml CH₂Cl₂, bromo-trimethyl-silane (310 μl, 2.4 mMol) is added and the mixture is stirred for 6 h at rt. It is concentrated in vacuo, the residue re-dissolved in MeOH and again concentrated. Trituration in tert-butyl-methyl-ether/CH₂Cl₂ and filtration yields the title compound as the hydrobromide salt: Anal. (+0.9 HBr+2 H₂O): C, H, N, Br; MS: [M+1]⁺=362; ¹H-NMR (DMSO-d₆): δ ppm 8.50 (d, 1H), 8.10 (d, 1H), 7.99 (d, 2H), 7.91 (d, 1H), 7.75 (m, 2H), 7.61 (t, 1 H), 7.48 (m, 2H), 7.28 (m, 2H), 7.00 (dd, 1H), 6.59 (dd, 1H); ³¹P-NMR (DMSO-d₆): δ 13.0 ppm.

The starting material is prepared as follows:

### Step 40.1: [(E)-2-(8-Hydroxy-quinolin-2-yl)-vinyl]-phosphonic acid diethyl ester

To a solution of 8-hydroxy-quinolin-2-carbaldehyde (4.8 g, 27.7 mMol) in 37 ml CH₂Cl₂ at −10° C., 37 ml of a 50% aq. solution of NaOH are given. Then (diethoxy-phosphorylmethyl)phosphonic acid diethyl ester (Fluka: tetraethyl methylenediphosphonate; 7 ml, 28 mMol) is added dropwise. After stirring the mixture for 4 h, the aq. layer is separated off and extracted twice with CH₂Cl₂. The organic layers are washed with water and brine, dried (Na₂SO₄), concentrated. Crystallization from DIPE/hexane gives the title compound: MS: [M+1]⁺=308.

### Step 40.2: [(E)-2-(8-Trifluoromethanesulfonyloxy-quinolin-2-yl)-vinyl]-phosphonic acid diethyl ester

[(E)-2-(8-Hydroxy-quinolin-2-yl)-vinyl]-phosphonic acid diethyl ester (1.29 g, 4.2 mMol) is dissolved in 26 ml CH₂Cl₂ and cooled to −78° C. Then Et₃N (1.75 ml, 12.6 mMol) is added, followed portion wise by (F₃CSO₂)₂O (1.04 ml, 6.3 mMol). After 1 h at −78° C., the mixture is poured into a mixture of EtOAc and water. The aq. phase is separated off and extracted twice with EtOAc. The organic layers are washed with water and brine, dried (Na₂SO₄) and concentrated. Chromatography (Combi Flash; CH₂Cl₂→CH₂Cl₂/EtOAc 4:1) gives the title compound: MS: [M+1]⁺=440; ¹H-NMR (DMSO-d₆): δ ppm 8.63 (d, 1H), 8.14 (d, 1H), 8.10 (d, 1H), 7.94 (d, 1H), 7.74 (t, 1H), 7.60 (dd, 1H), 7.24 (dd, 1H), 4.07 (quint, 2H₂C); 1.27 (t, 2H₃C).

### Step 40.3: [(E)-2-(8-Naphthalen-1-yl-quinolin-2-yl)-vinyl]-phosphonic acid diethyl ester

[(E)-2-(8-Trifluoromethanesulfonyloxy-quinolin-2-yl)-vinyl]-phosphonic acid diethyl ester (1.2 g, 2.73 mMol) dissolved in 84 ml tert.-butanol is degassed by repeated evacuation to HV and flushing with N₂. Then 1-naphthalene boronic acid (477 mg, 2.77 mMol), Pd(OAc)₂ (128 mg, 0.57 mMol), K₃PO₄ (1.452 g, 6.8 mMol) and 2-dicyclohexylphosphino-2′,4′,6′-triisopropylbiphenyl (XPhos; 323 mg, 0.68 mMol) are added. The reaction mixture is stirred for 2½ h at 82° C., diluted with water and EtOAc, the aq. phase separated off and extracted twice with EtOAc. The organic layers are washed with water and brine, dried (Na₂SO₄) and concentrated. The residue is adsorbed on SiO₂ put on top of a chromatography column (SiO₂) and the title compound eluted with CH₂Cl₂/EtOAc 4:1: MS: [M+1]⁺=418; TLC(CH₂Cl₂/EtOAc 4:1): R_{f}=0.17.

### EXAMPLE 41

### [2-(8-Naphthalen-1-yl-quinolin-2-yl)-ethyl]-phosphonic acid

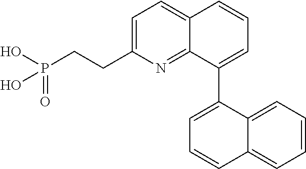

[2-(8-Naphthalen-1-yl-quinolin-2-yl)-ethyl]-phosphonic acid diethyl ester (200 mg, 0.48 mMol) is deprotected analogousely to Ex. 40 to the hydrobromide salt of the title compound: Anal. (+1.1 HBr+1.2; H₂O): C, H, N, Br; MS: [M+1]⁺=364.

The starting material is prepared as follows:

### Step 41. 1[2-(8-Naphthalen-1-yl-quinolin-2-yl)-ethyl]-phosphonic acid diethyl ester [(E)-2-(8-Naphthalen-1-yl-quinolin-2-yl)-vinyl]-phosphonic acid diethyl ester (0.48 g, 1.15 mMol) in 15 ml EtOH is hydrogenated in presence of 0.1 g Pd/C (10%; Engelhard 4505). The catalyst is filtered off, the filtrate concentrated and chromatographed (Combi Flash; CH₂Cl₂→EtOAc), giving the title compound: MS: [M+1]⁺=420; ¹H-NMR (DMSO-d₆): δ ppm 8.36 (d, 1H), 8.05 (d, 1H), 7.97 (d, 2H), 7.73 (d, 1H), 7.67 (t, 1H), 7.59 (dd, 1H), 7.46 (m, 3H), 7.27 (t, 1H), 7.23 (t, 1H), 3.71 (m, 2H₂C); 2.82 (m, H₂C), 1.74 (dt, H₂C), 1.03 (m, 2 H₃C).

### EXAMPLE 42

### [(E)-2-(6-Amino-8-naphthalen-1-yl-quinolin-2-yl)-vinyl]-phosphonic acid

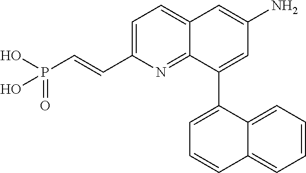

The title compound is obtained analogously to Ex. 17 after deprotection of [(E)-2-(6-amino-8-naphthalen-1-yl-quinolin-2-yl)-vinyl]-phosphonic acid diethyl ester (68 mg, 0.157 mMol) by bromo-trimethyl-silane (102 μl, 0.786 mMol) during 3 h at rt and purification by reversed phase chromatography as the TFA-salt: MS: [M+1]⁺=377; HPLC: t_{Ret}=0.69; ¹H-NMR (DMSO-d₆): δ ppm 8.08 (sb, 1H), 7.98 (d, 2H), 7.68 (sb, 1H), 7.60 (t, 1H), 7.48 (m, 1H), 7.44 (d, 1H), 7.33 (m, 2H), 7.18 (s, 1H), 6.96 (s, 1H), 6.91 (m, 1H), 6.40 (m, 1H).

The starting material is prepared as follows:

### Step 42.1: [(E)-2-(6-Nitro-8-naphthalen-1-yl-quinolin-2-yl)-vinyl]-phosphonic acid diethyl ester

To a suspension of 2-chloro-8-naphthalen-1-yl-6-nitro-quinoline (3.0 g, 8.96 mMol; Step 5.3) in 12 ml degassed dry DMF, diethyl vinylphosphonate (1.66 ml, 10.8 mMol), Et₃N (3.74 ml, 26.9 mMol), Pd(OAc)₂ (40.2 mg, 0.179 mMol) and Tri(o-tolyl)phosphine (109 mg, 0.358 mMol) are added. This mixture is stirred for 12 h at 100° C., cooled to rt and diluted with EtOAc and brine. The aq. phase is separated off and extracted twice with EtOAc. The organic layers are washed twice with water brine, dried (Na₂SO₄) and concentrated. Column chromatography (SiO₂; CH₂Cl₂→CH₂Cl₂/acetone 19:1→9:1) gives the title compound: MS: [M+1]⁺=463; HPLC: t_{Ret}=1.37; TLC(CH₂Cl₂/acetone 9:1): Rf=0.31.

### Step 42.2: [(E)-2-(6-Amino-8-naphthalen-1-yl-quinolin-2-yl)-vinyl]-phosphonic acid diethyl ester

[(E)-2-(6-Nitro-8-naphthalen-1-yl-quinolin-2-yl)-vinyl]-phosphonic acid diethyl ester (1.1 g, 2.38 mMol) is dissolved in 16.5 ml EtOH. Then 6 ml H₂O, Fe-powder (664 mg, 11.9 mMol) and NH₄Cl (636 mg, 11.9 mMol) are added. This mixture is stirred for 2 h at 65° C. and then diluted with 50 ml EtOAc and vigorously stirred. The aq. phase is separated off and extracted twice with EtOAc. The organic layers are washed twice with brine, dried (Na₂SO₄) and concentrated. Chromatography (Combi Flash; CH₂Cl₂/EtOAc 49:1→4:1) gives the title compound: MS: [M+1]⁺=433; HPLC: t_{Ret}=1.02; TLC(CH₂Cl₂/acetone 4:1): Rf=0.34.

### EXAMPLE 43

### [(E)-2-(6-Nitro-8-naphthalen-1-yl-quinolin-2-yl)-vinyl]-phosphonic acid

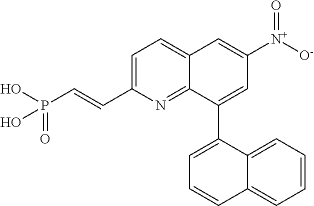

The title compound is obtained analogously to Ex. 17 after deprotection of [(E)-2-(6-nitro-8-naphthalen-1-yl-quinolin-2-yl)-vinyl]-phosphonic acid diethyl ester (97 mg, 0.21 mMol) by bromo-trimethyl-silane (136 μl, 1.05 mMol) during 4 h at rt: Anal. (+0.16 HBr+2 H₂O+0.2 hexane): C, H, N, Br; MS: [M+1]⁺=407; HPLC: t_{Ret}=1.08.

### EXAMPLE 44

### [(E)-2-(6-Methoxycarbonylamino-8-naphthalen-1-yl-quinolin-2-yl)-vinyl]-phosphonic acid

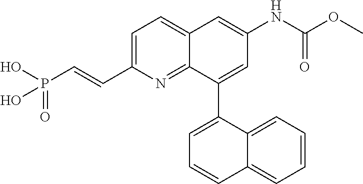

[(E)-2-(6-Methoxycarbonylamino-8-napthalen-1-yl-quinolin-2-yl)-vinyl]-phosphonic acid diethyl ester (45 mg, 0.092 mMol) is dissolved in 4.1 ml CH₂Cl₂. Then pyridine (14.7 μl, 0.206 mMol) and bromo-trimethyl-silane (59.5 μl, 0.46 mMol) are added. After stirring for 3 h at rt, pyridine (32.9 μl, 0.46 mMol) and 1 ml tert-butanol are added. Concentration and purification by reversed phase chromatography gives the title compound as the TFA-salt: Anal. (+0.72 TFA+1.2; H₂O): C, H, N, F; MS: [M+1]⁺=435; HPLC: t_{Ret}=0.81.

The starting material is prepared as follows:

### Step 44.1: [(E)-2-(6-Methoxycarbonylamino-8-naphthalen-1-yl-quinolin-2-yl)-vinyl]-phosphonic acid diethyl ester

A solution of [(E)-2-(6-amino-8-naphthalen-1-yl-quinolin-2-yl)-vinyl]-phosphonic acid diethyl ester (194 mg, 0.449 mMol) dissolved in 3 ml CH₂Cl₂ and 2 ml pyridine is cooled in an ice bath. Then methyl chloroformate (41.4 μl, 0.538 mMol) is added, warmed up to it and stirred for 1 h. The solution is diluted with 50 ml 5% aq. citric acid and 100 ml EtOAc, the aq. layer separated off and extracted twice with EtOAc. The organic layers are washed with water and brine, dried (Na₂SO₄) and concentrated. Chromatography (Combi Flash; CH₂Cl₂/acetone 99:1→9:1) and trituration in DIPE gives the title compound: MS: [M+1]⁺=491; HPLC: t_{Ret}=1.20.

### EXAMPLE 45

### [(E)-2-(6-Acetylamino-8-naphthalen-1-yl-quinolin-2-yl)-vinyl]-phosphonic acid

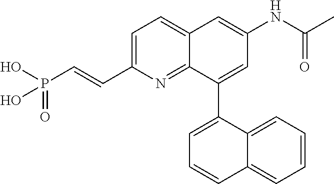

[(E)-2-(6-Acetylamino-8-naphthalen-1-yl-quinolin-2-yl)vinyl]-phosphonic acid diethyl ester (33 mg, 0.070 mMol) is deprotected as described in Ex. 44, yielding the title compound as the TFA-salt: Anal. (+0.6 TFA+1.2 H₂O): C, H, N, F; MS: [M+1]⁺=419; HPLC: t_{Ret}=0.73.

The starting material is prepared as follows:

### Step 45.1: [(E)-2-(6-Acetylamino-8-naphthalen-1-yl-quinolin-2-yl)-vinyl]-phosphonic acid diethyl ester

A solution of [(E)-2-(6-amino-8-naphthalen-1-yl-quinolin-2-yl)-vinyl]-phosphonic acid diethyl ester (194 mg, 0.449 mMol) dissolved in 3 ml CH₂Cl₂ and 2 ml pyridine is cooled in an ice bath. Then acetic anhydride (50.8 μl, 0.538 mMol) is added, warmed up to rt and stirred for 2 h. Workup as described in Step 44.1 gives the title compound: MS: [M+1]⁺=475; HPLC: t_{Ret}=1.11.

### EXAMPLE 46

### [(E)-2-(6-Methanesulfonylamino-8-naphthalen-1-yl-quinolin-2-yl)-vinyl]-phosphonic acid

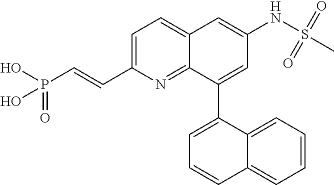

[(E)-2-(6-Methanesulfonylamino-8-napthalen-1-yl-quinolin-2-yl)-vinyl]-phosphonic acid diethyl ester (29 mg, 0.057 mMol) in 2.45 ml CH₂Cl₂ and pyridine (8.1 μl, 0.114 mMol) is deprotected with bromo-trimethyl-silane (37.9 μl, 0.28 mMol). After 2.5 h at rt, 20 μl pyridine, 2.5 ml CH₂Cl₂ and 0.8 ml tert-butanol are added. Then the mixture is concentrated in vacuo, the title compound purified by reversed phase chromatography and isolated as lyophilisate from dioxane as its TFA-salt: Anal. (+0.8 TFA+1.1 H₂O+0.3 dioxane): C, H, N, S, F; MS: [M+1]⁺=455; HPLC: t_{Ret}=0.76; IR [cm⁻¹]: 1611s, 1323m, 1153s, 970s.

The starting material is prepared as follows:

### Step 46.1: [(E)-2-(6-Methanesulfonylamino-8-naphthalen-1-yl-quinolin-2-yl)-vinyl]-phosphonic acid diethyl ester

A solution of [(E)-2-(6-amino-8-naphthalen-1-yl-quinolin-2-yl)-vinyl]-phosphonic acid diethyl ester (194 mg, 0.449 mMol) dissolved in 3 ml CH₂Cl₂ and 2 ml pyridine is cooled in an ice bath. Then methanesulfonic anhydride (94 mg, 0.54 mMol) is added and warmed up to rt. After 5 h, 24 h and 48 h, additional portions of 94 mg methanesulfonic anhydride are added. Then the mixture is stirred for 2 h at 50° C. and finally workup as described in Step 44.1. Chromatography (Combi Flash; CH₂Cl₂/acetone 99:1→9:1) gives {(E)-2-[6-(dimethanesulfonyl)amino-8-naphthalen-1-yl-quinolin-2-yl]-vinyl}-phosphonic acid diethyl ester A followed by the title compound B: A: MS: [M+1]⁺=589; HPLC: t_{Ret}=1.20. B: MS: [M+1]⁺=511; HPLC: t_{Ret}=1.12.

### EXAMPLE 47

### {(E)-2-[6-(di-methanesulfonyl)amino-8-naphthalen-1-yl-quinolin-2-yl]-vinyl}-phosphonic acid

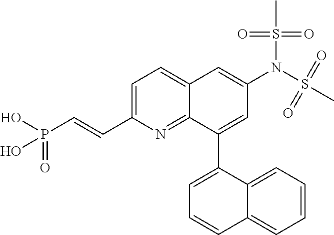

{(E)-2-[6-di-methanesulfonyl)amino-8-naphthalen-1-yl-quinolin-2-yl]-vinyl}-phosphonic acid diethyl ester (38 mg, 0.065 mMol) is deprotected as described in Ex. 46, yielding the title compound as the TFA-salt: Anal. (+0.6 TFA+0.4 H₂O+0.6 dioxane): C, H, N, S, F; MS: [M+1]⁺=533; HPLC: t_{Ret}=0.93; IR [cm⁻¹]: 1372s, 1162s, 977s, 937s.

### EXAMPLE 48

### ((E)-2-{8-Naphthalen-1-yl-6-[(pyridine-3-carbonyl)-amino]-quinolin-2-yl}-vinyl)phosphonic acid

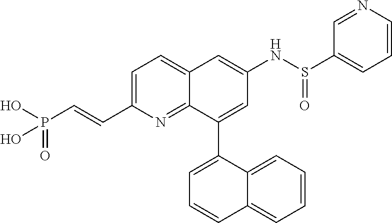

((E)-2-{8-Naphthalen-1-yl-6-[(pyridine-3-carbonyl)-amino]-quinolin-2-yl}-vinyl)-phosphonic acid diethyl ester (80 mg, 0.149 mMol) is deprotected as described in Ex. 44, yielding the title compound as the TFA-salt: Anal. (+1.1 TFA+1.3 H₂O): C, H, N, F, P; MS: [M+1]⁺=482.

The starting material is prepared as follows:

### Step 48.1: ((E)-2-{8-Naphthalen-1-yl-6-[(pyridine-3-carbonyl)-amino]-quinolin-2-yl}-vinyl)-phosphonic acid diethyl ester

To [(E)-2-(6-amino-8-naphthalen-1-yl-quinolin-2-yl)-vinyl]-phosphonic acid diethyl ester (155 mg, 0.359 mMol) dissolved in 5 ml DMF, nicotinic acid (48.6 mg, 0.395 mMol), Et₃N (749 μl, 5.38 mMol) and DMAP (19.2 mg, 0.157 mMol) are added. Then propylphosphonic anhydride (440 μl, 0.75 mMol) is dropped in and the resulting solution is stirred for 1 h. The reaction mixture is poured into water and EtOAc, the aq. layer separated off and extracted twice with EtOAc. The organic layers are washed with water and brine, dried (Na₂SO₄) and concentrated. Chromatography (Combi Flash; CH₂Cl₂/acetone 19:1→1:1) and trituration in DIPE gives the title compound: m.p.: 216-217° C.; MS: [M+1]⁺=538.

### EXAMPLE 49

### rac. {(E)-2-[6-(2-tert-Butoxycarbonylamino-3,3-dimethyl-butyrylamino)-8-naphthalen-1-yl-quinolin-2-yl]-vinyl}-phosphonic acid A and rac. {(E)-2-[6-(2-amino-3,3-dimethyl-butyrylamino)-8-naphthalen-1-yl-quinolin-2-yl]-vinyl}-phosphonic acid B

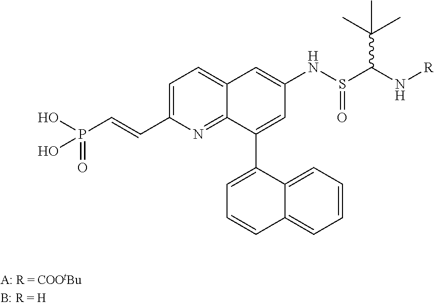

rac. {(E)-2-[6-(2-tert-Butoxycarbonylamino-3,3-dimethyl-butyrylamino)-8-naphthalen-1-yl-quinolin-2-yl]-vinyl}-phosphonic acid diethyl ester (67.5 mg, 0.106 mMol) suspended in 2.3 ml CH₂Cl₂ and pyridine (39.6 μl, 0.55 mMol) is cooled in an ice-bath. Then a solution of Me₃SiBr (68.5 μl, 0.53 mMol) in 2 ml CH₂Cl₂ is added and the mixture is stirred for 8 h at rt. After addition of a solution of 39.4 μl pyridine in 2.3 ml CH₂Cl₂ and 1 ml methanol, the mixture is concentrated in vacuo. Reversed phase chromatography and lyophilisation gives B as its TFA-salt, followed by A (bis TFA-salt): A: Anal. (+1.8 TFA+1 H₂O+0.6 dioxane): F; MS: [M+1]⁺=590. B: Anal. (+1.8 TFA+1 H₂O+0.6 dioxane): F; MS: [M+1]⁺=490.

The starting material is prepared as follows:

### Step 49.1: rac. {(E)-2-[6-(2-tert-Butoxycarbonylamino-3,3-dimethyl-butyrylamino)-8-naphthalen-1-yl-quinolin-2-yl]-vinyl}-phosphonic acid diethyl ester

[(E)-2-(6-amino-8-naphthalen-1-yl-quinolin-2-yl)-vinyl]-phosphonic acid diethyl ester (275 mg, 0.635 mMol) and rac. 2-tert-butoxycarbonylamino-3,3-dimethyl-butyric acid (161 mg, 0.698 mMol) are converted to the title compound as described in Step 48.1 (reaction time: 20 h rt, 4 h 60° C.): m.p.: 249-250° C.; MS: [M+1]⁺=646.

### EXAMPLE 50

### N-[8-Naphthalen-1-yl-2-(2H-tetrazol-5-yl)-quinolin-6-yl]-carbamic acid ethyl ester

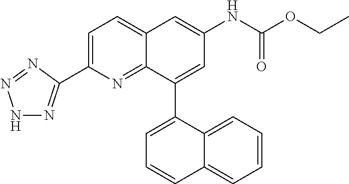

A mixture of sodium azide (228 mg, 3.5 mMol) and 0.2 ml toluene in a dried vessel is cooled in an ice-bath. Then 1.95 ml Et₂AlCl (1.8 M in toluene, 3.5 mMol) are added and the mixture is stirred for 5.5 h at rt. After cooling in an ice-bath, N-(2-cyano-8-naphthalen-1-yl-quinolin-6-yl)carbamic acid ethyl ester (100 mg, 0.27 mMol) is added during 20 min divided in 3 portions. After 40 h at rt, the mixture is poured into a mixture of EtOAc and 10% aq. citric acid. The aq. layer is separated off and extracted twice with EtOAc. The organic phases are washed with water and brine, dried (Na₂SO₄) and concentrated. Chromatography (Combi Flash; toluene/EtOAc 19:1→1:4) and trituration in DIPE gives the title compound: MS: [M+1]⁺=411; HPLC: t_{Ret}=1.17; TLC(toluene/EtOAc 1:3): R_{f}=0.36.

The starting material is prepared as follows:

### Step 50.1: 8-Naphthalen-1-yl-6-nitro-quinoline-2-carbonitrile

A mixture of 2-chloro-8-naphthalen-1-yl-6-nitro-quinoline (10 g, 29.8 mMol; Step 5.3) and CuCN (3.97 g, 44.3 mMol) in 60 ml NMP is heated up to 200° C. for 2 h by micro wave activation. Then the reaction mixture is diluted with water and EtOAc, the aq. layer separated off and extracted twice with EtOAc. The organic phases are washed with water and brine, dried (Na₂SO₄) and concentrated after addition of 45 g of SiO₂. The resulting powder is put on top of a chromatography column (SiO₂; CH₂Cl₂/hexane 1:1) and the title compound eluted with CH₂Cl₂/hexane 1:1→2:1: MS: [M−1]=324; HPLC: t_{Ret}=1.34; TLC(CH₂Cl₂/hexane 1:1): Rf=0.14.

### Step 50.2: 6-Amino-8-naphthalen-1-yl-quinoline-2-carbonitrile

To a suspension of 8-naphthalen-1-yl-6-nitro-quinoline-2-carbonitrile (5.3 g, 16.4 mMol) in 114 ml EtOH and 41 ml H₂O, NH₄Cl (4.39 g, 82 mMol) and iron powder (4.58 g, 82 mMol) are added. This mixture is stirred at 80° C. for 16 h, then filtered though Hyflo and the residue extensively washed with EtOAc. The aq. layer is separated off and extracted twice with EtOAc. The organic phases are washed with water and brine, dried (Na₂SO₄) and concentrated. Chromatography (Combi Flash; hexane CH₂Cl₂) gives the title compound: MS: [M+1]⁺=296; HPLC: t_{Ret}=1.17; TLC(CH₂Cl₂/acetone 30:1): Rf=0.57.

### Step 50.3: N-(2-Cyano-8-naphthalen-1-yl-quinolin-6-yl)-carbamic acid ethyl ester

6-Amino-8-naphthalen-1-yl-quinoline-2-carbonitrile (470 mg, 1.59 mMol) in 11 ml CH₂Cl₂ and 7 ml pyridine is cooled in an ice bath. Then ethyl chloroformate (190 μl, 2.0 mMol) is added, warmed up to rt and stirred for 1 h. The solution is diluted with 5% aq. citric acid and EtOAc, the aq. layer separated off and extracted twice with EtOAc. The organic layers are washed with water and brine, dried (Na₂SO₄) and concentrated. Chromatography (Combi Flash; CH₂Cl₂→CH₂Cl₂/EtOAc 19:1) gives the title compound: MS: [M+1]⁺=368; HPLC: t_{Ret}=1.30; TLC(CH₂Cl₂/EtOAc 20:1): Rf=0.6.

### EXAMPLE 51

### N-[8-Naphthalen-1-yl-2-(2H-tetrazol-5-yl)-quinolin-6-yl]-acetamide

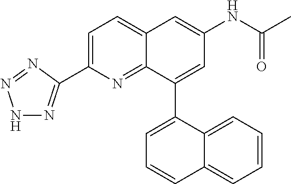

Prepared from N-(2-cyano-8-naphthalen-1-yl-quinolin-6-yl)-acetamide as described in Ex. 50: MS: [M+1]⁺=381; HPLC: t_{Ret}=1.00; TLC(CH₂Cl₂/EtOAc 1:2): Rf=0.07.

The starting material is prepared as follows:

### Step 51.1: N-(2-Cyano-8-naphthalen-1-yl-quinolin-6-yl)-acetamide

6-Amino-8-naphthalen-1-yl-quinoline-2-carbonitrile (200 mg, 0.68 mMol) in 4.6 ml CH₂Cl₂ and 3 ml pyridine is cooled in an ice bath. Then acetic acid anhydride (77 μl, 0.82 mMol) is added, warmed up to rt and stirred for 9 h. Work up and purification as described in Step 50.3 gives the title compound: MS: [M+1]⁺=338; HPLC: t_{Ret}=1.15; TLC(CH₂Cl₂/EtOAc 20:1): Rf=0.41.

### EXAMPLE 52

### 6-Thiophen-2-yl-8-naphthalen-1-yl-2-(2H-tetrazol-5-yl)-quinoline

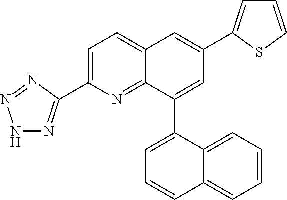

Prepared from 6-thiophen-2-yl-8-naphthalen-1-yl-quinoline-2-carbonitrile (112 mg, 0.31 mMol) as described in Ex. 50: MS: [M+1]⁺=406; HPLC: t_{Ret}=1.41; TLC(hexane/EtOAc 1:2): Rf=0.21.

The starting material is prepared as follows:

### Step 52.1: 6-Iodo-8-naphthalen-1-yl-quinoline-2-carbonitrile

6-Amino-8-naphthalen-1-yl-quinoline-2-carbonitrile (400 mg, 1.35 mMol) and pieces of ice in 8.4 ml conc. HCl are cooled to −15° C. Then a solution of NaNO₂ (186 mg, 2.7 mMol) in 11 ml H₂O is added dropwise and the mixture is stirred for 20 min. The suspension is added portion wise to an ice cooled solution of KI (12.1 g, 73 mMol) in 51 ml H₂O. After 3 h at rt, the mixture is diluted with EtOAc and water/sat. NaHCO₃ 1:1. The aq. phase is separated off and extracted twice with EtOAc. The organic layers are washed with water and brine, dried (Na₂SO₄) and concentrated. Chromatography (Combi Flash; hexane→CH₂Cl₂) gives the title compound: MS: [M+1]⁺=407; HPLC: t_{Ret}=1.49; TLC(CH₂Cl₂/hexane 8:1): R_{f}=0.6.

### Step 52.2: 6-Thiophen-2-yl-8-naphthalen-1-yl-quinoline-2-carbonitrile

A mixture of 6-iodo-8-naphthalen-1-yl-quinoline-2-carbonitrile (354 mg, 0.87 mMol), thiophen-2-boronic acid (133 mg, 1.04 mMol) and K₂CO₃ (1.91 ml, 1 M in H₂O) in 4.5 ml DMF is degassed by repeated evacuation by HV and flushing with N₂. Then (Ph₃P)₂PdCl₂ (35 mg, 0.05 mMol) is added. This mixture is stirred in a pre-heated oil bath of 110° C. for 40 min, cooled to rt and filtered. The filtrate is diluted with water and EtOAc, the aq. layer separated off and extracted twice with EtOAc. The organic layers are washed with water and brine, dried (Na₂SO₄) and concentrated. Chromatography (Combi Flash; hexane/CH₂Cl₂ 99:1→1:9) yields the title compound: MS: [M+1]⁺=363; HPLC: t_{Ret}=1.51; TLC(CH₂Cl₂/hexane 8:1): R_{f}=0.52.

### EXAMPLE 53

### 6-Thiophen-2-yl-2-(2-methyl-2H-tetrazol-5-yl)-8-naphthalen-1-yl-quinoline A and 6-thiophen-2-yl-2-(1-methyl-1H-tetrazol-5-yl)-8-naphthalen-1-yl-quinoline B

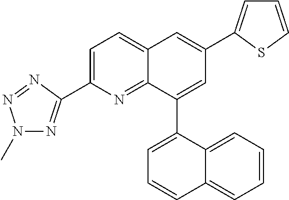

6-Thiophen-2-yl-8-naphthalen-1-yl-2-(2H-tetrazol-5-yl)-quinoline (65 mg, 0.16 mMol) is dissolved in 1 ml dioxane. Then Cs₂CO₃ (156 mg, 0.48 mMol) is added, followed by methyl-iodide (11.2 μl, 0.18 mMol). After 5 d at rt, another portion of 11.2 μl methyl-iodide is added. The mixture is stirred for 3 h and then diluted with EtOAc and water. The aq. layer is separated off and extracted twice with EtOAc. The organic phases are washed with water and brine, dried (Na₂SO₄) and concentrated. Chromatography (Combi Flash; hexane/toluene 1:5→hexane/toluene/EtOAc 2:10:1) and reversed phase chromatography gives B and A. A: MS: [M+1]⁺=420; HPLC: t_{Ret}=1.502; ¹H-NMR (DMSO-d₆): δ ppm 8.72 (d, 1H), 8.46 (s, 1H), 8.27 (d, 1H), 8.10 (s, 1H), 8.05 (d, 1H), 8.03 (d, 1H), 7.80 (d, 1H), 7.67 (m, 2H), 7.57 (d, 1H), 7.50 (t, 1H), 7.37 (d, 1H); 7.33 (t, 1H), 7.20 (t, 1H), 4.33 (s, H₃C). B: MS: [M+1]⁺=420; HPLC: t_{Ret}=1.562; ¹H-NMR (DMSO-d₆): δ ppm 8.78 (d, 1H), 8.52 (s, 1H), 8.34 (d, 1 H), 8.32 (s, 1H), 8.07 (d, 1H), 8.04 (d, 1H), 7.88 (d, 1H), 7.72 (d, 1H), 7.67 (t, 1H), 7.62 (d, 1H), 7.52 (m, 1H); 7.33 (m, 2H), 7.23 (dd, 1H), 3.17 (s, H₃C).

### EXAMPLE 54

### 6-Chloro-8-naphthalen-1-yl-quinoline-2-carboxylic acid

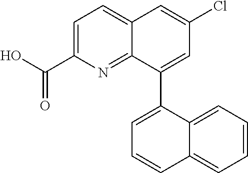

A mixture of 6-chloro-8-naphthalen-1-yl-quinoline-2-carbonitrile (41 mg, 0.13 mMol) and 2M NaOH (0.8 ml, 1.6 mMol) in 2.5 ml MeOH is refluxed for 2 h. The mixture is concentrated, quenched with 0.4 ml 4 M HCl (0.4 ml), diluted with water and extracted twice with dichloromethane. The organic layers are dried (Na₂SO₄) and concentrated. Chromatography (CH₂Cl₂/MeOH 2% to 7%) gives the title compound: MS: [M+1]⁺=334, 336 (Cl pattern); HPLC: t_{Ret}=1.37; TLC(CH₂Cl₂/MeOH 9:1): Rf=0.36; ¹H-NMR (DMSO-d₆): δ ppm 13.20 (br s, 1H), 8.35 (d, 1H), 8.10 (d, 1H), 8.02 (m, 2H), 7.81 (d, 1H), 7.62 (m, 1H), 7.52-7.48 (m, 2H), 7.34 (m, 1H), 7.25 (d, 2H).

The starting material is prepared as follows:

### Step 54.1: 2,6-Dichloro-8-naphthalen-1-yl-quinoline

Prepared from 2-bromo-4-chloroaniline in a similar manner as described in Step 4.1-4.2: MS: [M+1]⁺=324, 326 (2×Cl pattern); HPLC: t_{Ret}=1.54; TLC(hexane/EtOAc 1:1): R_{f}=0.63.

### Step 54.2: 6-Chloro-8-naphthalen-1-yl-quinoline-2-carbonitrile

A mixture of 2,6-dichloro-8-naphthalen-1-yl-quinoline (100 mg, 0.31 mMol) and CuCN (30 mg, 0.34 mMol) in 0.7 ml NMP is heated with microwave excitation for 10 min at 180° C., 20 min at 200° C. and 40 min at 210° C. The mixture is quenched with water and the precipitate is filtered. The solid is washed with water and dissolved in dichloromethane. The organic solution is washed with sat. NaHCO₃ and brine, dried (Na₂SO₄) and concentrated. Chromatography (hexane/EtOAc 5%→20%) gives the title compound: MS: [M+1]⁺=315, 317 (Cl pattern); HPLC: t_{Ret}=1.45; TLC(hexane/EtOAc 1:1): R_{f}=0.55.

### FPPS Enzyme Assay

| | | |
|---|---|---|
| Abreviations used: | SPA | Scintillation Proximity Assay |
| | FPPS | Farnesyl pyrophosphate synthase |
| | FPP | Farnesyl pyrophosphate |
| | IPP | Isopentenyl pyrophosphate |
| | GPP | Geranyl pyrophosphate |
| | DMAPP | Dimethyl allyl pyrophosphate |
| | FlashPlate ™ | Scintillating microtiter plate |

All steady-state kinetic parameters are determined by fitting to the Henri-Michaelis-Menten equation using the non-linear regression algorithm of GraphPad Prism software (GraphPad Prism version 4.00 for Windows, GraphPad Software, San Diego Calif. USA),

V=Vₘₐₓ [S]/[S]+Kₘ

where Vₘₐₓ equals the maximal rate of product formation over time; [S]=the concentration of IPP or GPP; Kₘ=the Henri-Michaelis-Menten constant which is includes factors for affinity and catalytic rate. K_{cat} is determined by Vₘₐₓ/[FPPS]; IC₅₀ curves are fit to a variable slope, sigmoidal curve using non-linear regression algorithm in GraphPad Prism software as

Y=bottom+(top−bottom)/1+10^{(log IC50-X)×Hill Slope}.

Recombinant human Farnesyl Pyrophosphate synthase (FPPS) was cloned, expressed and purified as previously described {J.-M. Rondeau et al., ChemMedChem 2006, 1, 267-271.} and stored as a 10 mg/mL stock solution in 25 mM Tris pH 7.4, 25 mM NaCl, 2 mM DTT (dithiothreitol). Geranyl pyrophosphate (GPP) was purchased from Anawa AG (Switzerland) and stored as a 1 mg/mL solution in 4 parts isopropanol:3 parts ammonia:1 part water. 1-[³H]Isopentenyl pyrophosphate (IPP), 50 Ci/mmol; 1 Ci/mL, was purchased from Anawa AG and stored in ethanol:ammonia hydroxide 1:1 at −80 C. 1-[³H] Farnesyl pyrophosphate triammonium salt, 100 Ci/mmol; 1 mCi/mL in 70% ethanol, 0.25 M ammonium bicarbonate was purchased from Anawa AG. Phospholipid-coated 384-well image FlashPlates™ were purchased from PerkinElmer. the assay buffer consisted of 20 mM HEPES pH7.4, 5 mM MgCl₂ and 1 mM CaCl₂.

The FPPS assay is performed in a final detection volume of 12 μl under steady-state conditions as follows:

To the lipid-coated flashplate, note: LEADseeker (trademark) should be spelled consistently. FlashPlates (trademark) should be spelled consistently throughout.

3 μl of test compound solution in 18% DMSO/water or 18% DMSO/assay buffer (carrier control) (end concentration of DMSO in the assay 4.5%),

3 μl of GPP working solution, final concentration 150 nM

3 μl of [³H]-IPP working solution final concentration 150 nM

3 μl of FPPS working solution are added, final concentration 500 pM.

All components are diluted in assay buffer. After addition of all components (in the order listed above), the mixture is incubated for 45 minutes at room temperature.

The inhibition of the FPPS enzymatic reaction by compounds is measured, in a LEADseeker IV (Amersham Biotech), reader, reading time 2 min, method SPA, using for flat field correction the Amersham 384-well standard and quasi-coincident radiation correction, is used.

Test compounds are arrayed in an 8 or 16 point, 2 or 3-fold serial dilution series in 90% DMSO such that the highest concentration is 2 mM in 90% DMSO. In order to obtain replicate data, these compound source plates are diluted and replicated into 384 well image FlashPlates (using a CyBiWell HTS pipetter) to contain 3 μL of compound solution each, to which the assay reagents are added and read. This procedure results in a dose response curve performed in triplicate with 100 μM being the highest concentration tested.

As positive control, Zometa can be used, which inhibits the reaction with an IC50 of between 50 and 200 nM. Selected compounds were assayed in an antibody based assay as described above; results are summarized in the following table.

| | FPPS | | FPPS |
|---|---|---|---|
| Ex. | IC₅₀ [μM] | Ex. | IC₅₀ [μM] |
| 1 | 0.52 | 10.1 | 2.8 |
| 2.a.ii | 6.5 | 11 | 7.9 |
| 2.b.i | >10 | 11.6 | >100 |
| 2.c.i | >10 | 12 | >100 |
| 2.c.ii | 2.7 | 13 | 13.5 |
| 2.d.i | >10 | 13.2 | 100 |
| 2.g.i | 19.7 | 13.3 | 100 |
| 2.g.ii | >100 | 14 | 39.7 |
| 3 | 40.3 | 15.a | >100 |
| 5 | 4.1 | 15.b | 54.3 |
| 6 | 14 | 36 | 0.14 |
| 9 | 21.5 | 40 | 0.007 |
| 9.1 | >100 | 41 | 2.3 |
| 10 | 2.8 | 54 | 0.34 |

Selected compounds are assayed in another FPPS based assay:

### FPPS Assay: LC-MS Method

Into each well of a 384-well plate, 5 μL of compound in 20% DMSO/water is placed. 25 μL of GPP/IPP (5 μM each in assay buffer) is then added. The reaction is started with the addition of 10 μL of FPPS (diluted 1 to 10000 with assay buffer). After 10 minutes, the reaction is stopped by addition of 10 μL of 2 μM FSPP in 2% DMIPA/IPA. The reaction mixture is then extracted with 50 μL of n-pentanol using vortex mixing. After phase separation, 25 μL of the upper (n-pentanol) layer is transferred to a new 384-well plate and the pentanol is evaporated using a vacuum centrifuge. The dried residue is reconstituted in 50 μL of 0.1% DMIPA/water for analysis by the LC/MS/MS method.

| | | |
|---|---|---|
| Abbreviations used: | assay buffer | 20 mM HEPES, 5 mM MgCl₂ |
| | | and 1 mM CaCl₂ |
| | DMIPA | Dimethylisopropylamine |
| | FPPS | Farnesyl pyrophosphate synthase |
| | FSPP | Farnesyl S-thiolopyrophosphate |
| | IPA | Isopropanol |
| | IPP | Isopentenyl pyrophosphate |
| | GPP | Geranyl pyrophosphate |

### LC/MS/MS Analytical Method

LC/MS/MS analyses are performed on a Micromass Quattro Micro interfaced to an Agilent 1100 binary LC pump. Injection is performed by a Gilson 215/889 autosampler using an injection loop size of 2.5 μL. Chromatography is performed on a Waters 2.1×20 mm Xterra MS C18 5 μm guard column (P/N186000652) contained in a guard column holder (P/N 186000262) using 0.1% DMIPA/methanol as solvent A and 0.1% DMIPA/water as solvent B. The gradient is 5% A from 0.00 to 0.30 min, 50% A at 0.31 min, 80% A at 1.00 min, and 5% A from 1.01 to 2.00 min. The flow rate is 0.3 mL/min and the flow is diverted to waste from 0.00 to 0.50 min and again from 1.20 to 2.00 min.

The MRM transitions monitored are 381⁻>79⁻ for FPP and 397⁻>159⁻ for FSPP at a collision energy of 22 eV and a collision cell pressure of 2.1×10⁻³ mbar of Ar. The dwell time per transition is 400 msec with a span of 0.4 Da. The interchannel delay and interscan delay are both 0.02 sec. Other mass spectrometric operating parameters are: capillary, 2.0 kV; cone, 35 V; extractor, 2.0 V, source temp., 100° C.; desolvation gas temp., 250° C.; desolvation gas flow, 650 L/hr; cone gas flow, 25 L/hr; multiplier, 650 V.

The total cycle time per sample is 2.5 minutes. Since the analysis is formatted for 384-well plates, a plate is analyzed in 16 hours. The chromatograms are processed using Quanlynx software, which divides the area of individual FPP peaks by the area of the FSPP peaks (internal standard). The resulting values are reported as the relative response for the corresponding sample well.

The results of LC-MS the assay are summarized in the following table.

| | FPPS | | FPPS |
|---|---|---|---|
| Ex. | IC₅₀ [μM] | Ex. | IC₅₀ [μM] |
| 42 | 0.009 | 47 | 0.204 |
| 48 | 0.012 | 27c.ii. | 0.205 |
| 44 | 0.014 | 38 | 0.217 |
| 50 | 0.019 | 17 | 0.266 |
| 30 | 0.024 | 39 | 0.267 |
| 45 | 0.026 | 35 | 0.311 |
| 29 | 0.037 | 21 | 0.507 |
| 51 | 0.048 | 49 | 0.570 |
| 33 | 0.056 | 28 | 0.606 |
| 31 | 0.069 | 26 | 0876 |
| 46 | 0.091 | 27d.ii | 0.900 |
| 34 | 0.100 | 27f.ii | 0.977 |
| 43 | 0.103 | 32 | 0.160 |

The compounds of the present invention show FPPS inhibition in the range of 1 nM to >100 μM, preferably from 1 nM to 50 μM, more preferably from 1 nM to 10 μM, more preferably from 1 nM to 1 μM, more preferably from 1 nM to 900 nM, more preferably from 1 nM to 800 nM, more preferably from 1 nM to 700 nM, more preferably from 1 nM to 600 nM, more preferably from 1 nM to 500 nM, more preferably from 1 nM to 400 nM, more preferably from 1 nM to 300 nM, more preferably from 1 nM to 200 nM, more preferably from 1 nM to 100 nM, more preferably from 1 nM to 90 nM, more preferably from 1 nM to 80 nM, more preferably from 1 nM to 70 nM, more preferably from 1 nM to 60 nM, more preferably from 1 nM to 50 nM, such as from 1 nM to 40 nM, from 1 nM to 30 nM, from 1 nM to 20 nM, from 1 nM to 10 nM.

1. A compound of formula (I) 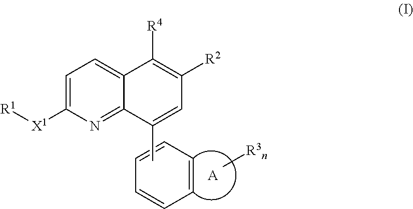
wherein
A represents an aryl, cycloalkyl, or heterocyclyl condensed to a phenyl ring;
R¹ represents a substituent different from hydrogen;
R² represents hydrogen, halogen, nitro, optionally substituted amino, optionally substituted aryl, or optionally substituted heterocyclyl;
R³ represents oxo (═O), amino, or optionally substituted alkyl;
R⁴ represents hydrogen, or alkoxy
X¹ represents a direct bond or an alkanediyl which is optionally interrupted by one or more groups selected from —O—, —C(O)—, —N(H)—, —N(lower alkyl)-, alkenediyl, provided that when more than one of said groups is present, two or more oxygen or nitrogen atoms are not bonded directly together;
n represents an integer from 0-3;
with the proviso that the compounds 2-methyl-8-naphthalen-quinoline and 2,2′-dimethyl-[8,8′]-biquinolinyl are excluded;
or a pharmaceutically acceptable salt thereof.

2. A compound according to claim 1, wherein
A represents—together with the phenyl ring to which it is attached—a moiety selected from the group consisting of naphthalene, 1,2,3,4-tetrahydronaphthalene, indole, isoindole, quinoline, isoquinoline, aryl, cycloalkyl, and heterocyclyl condensed to the phenyl ring; said moiety optionally substituted by one or more substituents selected from the group consisting of lower alkyl, hydroxyl, and oxo;
R¹ represents one of the following groups: 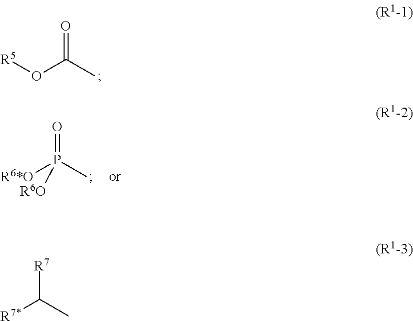
wherein
R⁵ represents hydrogen, unsubstituted alkyl, or alkyl substituted by aryl;
R⁶ represents hydrogen, or unsubstituted alkyl;
R⁶* represents hydrogen, or unsubstituted alkyl;
R⁷ represents hydrogen, halogen, hydroxy, amino, N-substituted amino, or N,N-di-substituted amino and R⁷* represents hydrogen, carboxy, alkoxycarbonyl or
R⁷* and R⁷ represent together with the carbon to which they are attached is an optionally substituted heterocycle;
R² represents hydrogen, chloro, bromo, iodo, nitro, amino, N-substituted amino, or N,N-disubstituted amino, the substituents being selected from the group consisting of (C₁-C₄)-alkylcarbonyl, (C₁-C₄)-alkoxycarbonyl, aryl, heteroarylcarbonyl, benzoxycarbonyl, (C₁-C₄)-alkylsulfonyl and (C₁-C₆)-alkylcarbonyl wherein the alkyl of the (C₁-C₆)-alkylcarbonyl is substituted by NH₂, (C₁-C₄)-alkyl or (C₁-C₄)-alkoxycarbonyl;
or R² represents an unsubstituted or substituted aryl, the substituents being selected from the group consisting of halo, cyano, hydroxy, lower alkyl, lower haloalkyl, lower alkyl substituted by aryl, lower alkoxy, lower alkoxy substituted by aryl, lower alkanoyl, lower alkoxycarbonyl, tri(lower alkyl)silyl;
or unsubstituted and substituted heterocyclyl, said heterocyclyl being mono- or bicyclic with 5 to 10 ring atoms wherein one to four ring atoms are selected from the group consisting of nitrogen, oxygen and sulfur, said substituents being selected from the group consisting of halo, cyano, hydroxy, lower alkyl, lower haloalkyl, lower alkyl substituted by aryl, lower alkoxy, lower alkoxy substituted by aryl, lower alkanoyl, lower alkoxycarbonyl, tri(lower alkyl)silyl, and oxo;
R³ represents oxo, amino, lower alkyl, substituted lower alkyl, the substituents being selected from the group consisting of hydroxyl, lower alkanoyl, and lower alkanoyloxy;
R⁴ represents hydrogen, or lower alkoxy;
X¹ represents a direct bond or a straight-chain or branched-chain C₁₋₁₂ alkandiyl which is optionally interrupted by one or more groups selected from —O—, —C(O)—, —N(H)—, —N(lower alkyl)-, or a straight-chain or branched-chain C₂₋₆ alkendiyl;
n represents 0 or 1;
or a pharmaceutically acceptable salt thereof.

3. A compound according to claim 1, wherein
A represents—together with the phenyl ring to which it is attached—a moiety selected from the group consisting of naphthalene, alpha or beta tetralone, indole, oxindole, quinoline, 2-(1H)quinolinone, isoquinoline, and 1-(2H)isoquinolinone;
R¹ represents (R¹-1), (R¹-2), or (R¹-3);
R² represents hydrogen, iodo, chloro, nitro,
amino, N-substituted amino, or N,N-disubstituted amino, the substituents being selected from the group consisting of ethoxycarbonyl, methylsulfonyl and unsubstituted or substituted phenyl, the substituents being selected from the group consisting of hydroxy, methyl, ethyl, iso-propyl, tert.-butyl, trifluoromethyl, benzyl, methoxy, ethoxy, iso-propoxy, tert.-butoxy, benzoxy, acyl, methoxycarbonyl, ethoxycarbonyl, iso-propoxycarbonyl, tert.-butoxycarbonyl, trimethylsilyl, unsubstituted heterocyclyl, and heterocyclyl substituted by one or two substitutents, said heterocyclyl being selected from the group consisting of pyrrole, pyridine, pyrimidine, indole, isoindole, furane, thiophene, and 1,3-benzodioxole, said substituents being selected from the group consisting of hydroxy, methyl, ethyl, iso-propyl, tert.-butyl, trifluoromethyl, benzyl, methoxy, ethoxy, iso-propoxy, tert.butoxy, benzoxy, acyl, methoxycarbonyl, ethoxycarbonyl, iso-propoxycarbonyl, tert.-butoxycarbonyl, trimethylsilyl, and oxo;
R³ represents oxo, amino, methyl, ethyl, propyl, n-butyl, sec-butyl, isobutyl, tert-butyl, 2,2-dimethylpropyl, 1,2,2-trimethylpropyl, 1-ethyl-propyl, substituted methyl, ethyl, propyl, n-butyl, sec-butyl, isobutyl, tert-butyl, 2,2-dimethylpropyl, 1,2,2-trimethylpropyl, or 1-ethyl-propyl, the substituents being selected from the group consisting of hydroxy, lower acetyl, propanoyl, butyroyl, acetyloxy, propanoyloxy, and butyroyloxy;
R⁴ represents hydrogen;
R⁵ represents hydrogen, lower alkyl, or lower alkyl substituted by phenyl;
R⁶ represents hydrogen, or lower alkyl;
R⁷ represents hydrogen, halogen, hydroxy, amino, N-substituted amino, or N,N-di-substituted amino, the substituents being selected from the group consisting of (C₁-C₄)-alkoxycarbonyl, benzoxycarbonyl, aminosulfonyl, (C₁-C₄)-alkoxycarbonyl-aminosulfonyl, and benzoxycarbonyl-aminosulfonyl, and R⁷* represents hydrogen, carboxy, or (C₁-C₄)-alkoxycarbonyl or
R⁷* and R⁷ together with the carbon to which they are attached represents a heterocycle optionally substituted by one or two oxo groups;
X¹ represents a direct bond, —CH═CH— or an alkandiyl selected from the group consisting of methandiyl, and 1,2-ethanediyl, where said alkandiyl is optionally interrupted by one or more groups selected from —C(O)—, —N(H)—;
n represents 0 or 1;
or a pharmaceutically acceptable salt thereof.

4. A compound according to claim 1 selected from the group consisting of:
8-Naphthalen-1-yl-quinoline-2-carboxylic acid;
{[(8-Naphthalen-1-yl-quinoline-2-carbonyl)-amino]-methyl}-phosphonic acid;
(8-Naphthalen-1-yl-quinolin-2-yl)-phosphonic acid;
[(8-Naphthalen-1-yl-quinolin-2-ylamino)-methyl]-phosphonic acid;
[(6-Nitro-8-naphthalen-1-yl-quinolin-2-ylamino)-methyl]-phosphonic acid;
[(6-Amino-8-naphthalen-1-yl-quinolin-2-ylamino)-methyl]-phosphonic acid;
[(6-Iodo-8-naphthalen-1-yl-quinolin-2-ylamino)-methyl]-phosphonic acid;
{[8-Naphthalen-1-yl-6-(1H-pyrrol-3-yl)-quinolin-2-ylamino]-methyl}-phosphonic acid;
[(8-Naphthalen-1-yl-6-pyridin-3-yl-quinolin-2-ylamino)-methyl]-phosphonic acid;
(8-Naphthalen-1-yl-quinolin-2-ylmethyl)-phosphonic acid;
{[(8-Naphthalen-1-yl-quinolin-2-ylmethyl)-amino]-methyl}-phosphonic acid;
rac. 2-[(tert-Butoxycarbonylamino-sulfonyl)-amino]-3-[(8-naphthalen-1-yl-quinoline-2-carbonyl)-amino]-propionic acid methyl ester;
rac. 2-[(Amino-sulfonyl)-amino]-3-[(8-naphthalen-1-yl-quinoline-2-carbonyl)-amino]-propionic acid methyl ester;
rac. 8-Naphthalen-1-yl-quinoline-2-carboxylic acid (1,1,4-trioxo-1lambda*6*-[1,2,5]thiadiazolidin-3-ylmethyl)-amide;
rac. 2-[(amino-sulfonylyamino]-3-[(8-naphthalen-1-yl-quinoline-2-carbonyl)-amino]-propionic acid;
[6-(3-Methoxy-phenyl)-8-naphthalen-1-yl-quinolin-2-yl]-phosphonic acid;
(8-Naphthalen-1-yl-6-thiophen-2-yl-quinolin-2-yl)-phosphonic acid;
(8-Naphthalen-1-yl-6-nitro-quinolin-2-yl)-phosphonic;
(8-Naphthalen-1-yl-6-nitro-quinolin-2-yl)-phosphonic acid monoethyl ester;
(6-Amino-8-naphthalen-1-yl-quinolin-2-yl)-phosphonic acid;
(6-Iodo-8-naphthalen-1-yl-quinolin-2-yl)phosphonic acid;
2-(8-Naphthalen-1-yl-2-phosphono-quinolin-6-yl)-pyrrole-1-carboxylic acid tert-butyl ester;
[8-Naphthalen-1-yl-6-(1H-pyrrol-2-yl)-quinolin-2-yl]-phosphonic acid;
[6-(1H-Indol-2-yl)-8-naphthalen-1-yl-quinolin-2-yl]-phosphonic acid;
[6-(6-Methoxy-pyridin-3-yl)-8-naphthalen-1-yl-quinolin-2-yl]-phosphonic acid;
[6-(6-hydroxy-pyridin-3-yl)-8-naphthalen-1-yl-quinolin-2-yl]-phosphonic acid;
[8-Naphthalen-1-yl-6-(1H-pyrrol-3-yl)-quinolin-2-yl]-phosphonic acid;
[6-(3-Hydroxy-phenyl)-8-naphthalen-1-yl-quinolin-2-yl]-phosphonic acid;
6-Ethoxycarbonylamino-8-naphthalen-1-yl-quinoline-2-carboxylic acid;
6-Amino-5-ethoxy-8-naphthalen-1-yl-quinoline-2-carboxylic acid;
6-Amino-8-naphthalen-1-yl-quinoline-2-carboxylic acid;
6-Iodo-8-naphthalen-1-yl-quinoline-2-carboxylic acid lithium salt;
6-Iodo-8-naphthalen-1-yl-quinoline-2-carboxylic acid;
5-Ethoxy-6-iodo-8-naphthalen-1-yl-quinoline-2-carboxylic acid lithium salt;
5-Ethoxy-6-iodo-8-naphthalen-1-yl-quinoline-2-carboxylic acid;
8-Naphthalen-1-yl-6-thiophen-3-yl-quinoline-2-carboxylic acid;
8-Naphthalen-1-yl-6-thiophen-2-yl-quinoline-2-carboxylic acid lithium salt;
8-Naphthalen-1-yl-6-thiophen-2-yl-quinoline-2-carboxylic acid;
8-Naphthalen-1-yl-6-pyrrol-2-yl-quinoline-2-carboxylic acid;
8-(5-Hydroxymethyl-naphthalen-1-yl)-quinoline-2-carboxylic acid;
8-(5-Methyl-naphthalen-1-yl)-quinoline-2-carboxylic acid;
8-(5-Amino-naphthalen-1-yl)-quinoline-2-carboxylic acid;
[(E)-2-(8-Naphthalen-1-yl-quinolin-2-yl)-vinyl]-phosphonic acid;
[2-(8-Naphthalen-1-yl-quinolin-2-yl)ethyl]-phosphonic acid;
[(E)-2-(6-Amino-8-naphthalen-1-yl-quinolin-2-yl)-vinyl]-phosphonic acid;
[(E)-2-(6-Nitro-8-naphthalen-1-yl-quinolin-2-yl)-vinyl]-phosphonic acid;
[(E)-2-(6-Methoxycarbonylamino-8-naphthalen-1-yl-quinolin-2-yl)-vinyl]-phosphonic acid;
[(E)-2-(6-Acetylamino-8-naphthalen-1-yl-quinolin-2-yl)-vinyl]-phosphonic acid;
[(E)-2-(6-Methanesulfonylamino-8-naphthalen-1-yl-quinolin-2-yl)-vinyl]-phosphonic acid;
{(E)-2-[6-(di-methanesulfonyl)amino-8-naphthalen-1-yl-quinolin-2-yl]-vinyl}-phosphonic acid;
((E)-2-{8-Naphthalen-1-yl-6-[(pyridine-3-carbonyl)-amino]-quinolin-2-yl}-vinyl)phosphonic acid;
rac. {(E)-2-[6-(2-tert-Butoxycarbonylamino-3,3-dimethyl-butyrylamino)-8-naphthalen-1-yl-quinolin-2-yl]-vinyl}-phosphonic acid;
rac. {(E)-2-[6-(2-amino-3,3-dimethyl-butyrylamino)-8-naphthalen-1-yl-quinolin-2-yl]-vinyl}-phosphonic acid
N-[8-Naphthalen-1-yl-2-(2H-tetrazol-5-yl)-quinolin-6-yl]-carbamic acid ethyl ester;
N-[8-Naphthalen-1-yl-2-(2H-tetrazol-5-yl)-quinolin-6-yl]-acetamide;
6-Thiophen-2-yl-8-naphthalen-1-yl-2-(2H-tetrazol-5-yl)-quinoline;
6-Thiophen-2-yl-2-(2-methyl-2H-tetrazol-5-yl)-8-naphthalen-1-yl-quinoline;
6-Thiophen-2-yl-2-(1-methyl-1H-tetrazol-5-yl)-8-naphthalen-1-yl-quinoline; and
6-Chloro-8-naphthalen-1-yl-quinoline-2-carboxylic acid.

5. A compound according to claim 1 of formula (II), or a pharmaceutically acceptable salt thereof 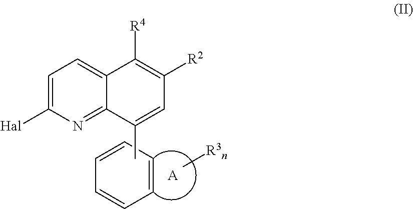
wherein A, R², R³, R⁴, n are as defined in claim 1; and Hal represents halogen.

6. A compound according to claim 1 of formula (IIX), or a pharmaceutically acceptable salt thereof 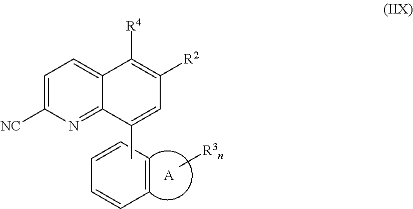
wherein A, R², R³, R⁴, n are as defined in claim 1.

7. A compound of the formula (I′) 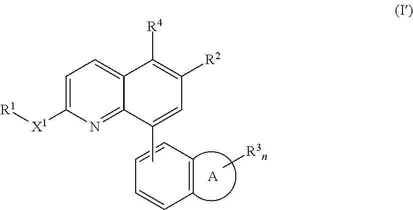
wherein
A represents an aryl, cycloalkyl, or heterocyclyl condensed to the phenyl ring;
R¹ represents a substituent different from hydrogen;
R² represents hydrogen, halogen, nitro, optionally substituted amino, optionally substituted aryl, or optionally substituted heterocyclyl;
R³ represents oxo (═O), amino, or optionally substituted alkyl;
R⁴ represents hydrogen, or alkoxy;
X¹ represents a direct bond or an alkanediyl which is optionally interrupted by one or more groups selected from —O—, —C(O)—, —N(H)—, —N(lower alkyl)-, alkenediyl; and
n represent an integer from 0-3;
or a pharmaceutically acceptable salt thereof.

8-10. (canceled)

11. A method for the treatment of a FPPS-dependent disease comprising the step of administering to a subject in need thereof a therapeutically effective amount of a compound of formula (I) according to claim 1, or in pharmaceutically acceptable salt form.

12. A pharmaceutical composition comprising a therapeutically effective amount of a compound of formula (I) according to claim 1, or in pharmaceutically acceptable salt form; and one or more pharmaceutically acceptable carrier material(s) or diluents.

13-16. (canceled)
